(19) United States
(12) Patent Application Publication
(10) Pub. No.: US 2022/0096654 A1
(43) Pub. Date: Mar. 31, 2022

(54) PYRROLOBENZODIAZEPINE-ANTIBODY CONJUGATES AND USES THEREOF

(71) Applicants: ADC THERAPEUTICS SA, Epalinges (CH); MEDIMMUNE LIMITED, Cambridge, MA (US)

(72) Inventors: Patricius Hendrikus Cornelis van Berkel, Epalinges (CH); Persis Amrolia, London (GB); Kerry Chester, Chester (GB); Jenny Yeung, London (GB)

(21) Appl. No.: 17/481,918

(22) Filed: Sep. 22, 2021

(30) Foreign Application Priority Data
Sep. 25, 2020 (GB) GB2015226.0

(51) Int. Cl. A61K 47/68; C07D 487/04

(52) U.S. Cl. CPC A61K 47/6889; C07D 487/04; A61K 47/6835; A61K 47/6803

(57) ABSTRACT

The present disclosure relates to novel pyrrolobenzodiazepine antibody-drug conjugates (PBD-ADCs) and therapeutic uses thereof.

### EARLIER APPLICATION

This application claims priority from United Kingdom application number GB2015226.0, filed 25 Sep. 2020. The priority application is hereby incorporated by reference in its entirety and for any and all purposes as if fully set forth herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 30,533 Byte ASCII (Text) file named “39852-201_5 T25,” created on Sep. 22, 2021.

### FIELD OF THE INVENTION

The present disclosure relates to novel pyrrolobenzodiazepine antibody-drug conjugates (PBD-ADCs) and therapeutic uses thereof.

### BACKGROUND TO THE INVENTION

Hematopoietic stem cell transplantation (HSCT) is a powerful treatment modality that enables replacement of host hematopoietic stem cells (HSCs) with HSCs from a healthy donor or genetically improved/corrected HSCs from the patient. This procedure often results in life-long benefits and can curatively treat many malignant and non-malignant blood and immune diseases. Over 1,000,000 patients have been transplanted in the last 60+ years for a wide range of blood and immune diseases, including leukemias, hemoglobinopathies, metabolic diseases and immunodeficiencies.

However, despite its broad therapeutic potential, HSCT is currently mainly restricted to otherwise incurable malignant diseases and it is estimated that fewer than 25% of patients that could benefit from HSCT undergo transplantation. This is largely due to undesirable morbidity/mortality from cytotoxic chemotherapy and irradiation-based conditioning currently necessary to enable successful donor HSC engraftment and the risks associated with graft versus host disease (GvHD). Traditionally, conditioning involves total body irradiation (TBI) and/or various chemotherapy prior to HSCT. These agents have been thought essential to make “space” in host bone marrow (BM) for donor HSC engraftment, but they are non-specific and induce significant collateral damage. Due to their non-specific nature, classic conditioning regimens lead to both detrimental short-term and long-term complications including multi-organ damage, mucositis, need for frequent red blood cell and platelet transfusions, infertility, and secondary malignancies. Additionally, these agents result in profound and prolonged immune ablation, which predisposes patients to serious and sometimes fatal opportunistic infections necessitating extended hospitalizations and exposure to toxic side effects of anti-infective agents. Although much work has led to the development of reduced intensity conditioning (RIC) methods, which use lower dose combination chemotherapy with or without low dose irradiation, patients still experience many of these debilitating side effects. Eliminating such harsh conditioning regimens would dramatically improve HSCT and expand its use.

One more recent approach has been to use antibodies that target HSCs. Numerous candidate target molecules discussed in the literature, including CD13, CD33, CD34, CD44, CD45, CD49d: VLA-4, CD49f: VLA-6, CD59, CD84: CD150 family, CD90: Thyl, CD93, CD105: Endoglin, CD117: cKit/SCF receptor, CD123: IL-3R, CD126: IL-6R, CD133, CD135: Flt3 receptor, CD166: ALCAM, CD184: CXCR4, Prominin 2, Erythropoietin R, Endothelial Cell—Selective Adhesion Molecule, CD244, TieI, Tie2, MPL, G-CSFR or CSF3R, IL-1R, gp130, Leukemia inhibitory factor Receptor, oncostatin M receptor, Embigin and IL-18R.

### SUMMARY OF THE INVENTION

The instant disclosure is based, at least in part, on the finding that anti-CD45 antibodies combined with a pyrrolobenzodiazepine cytotoxin, in the form of an antibody drug conjugate, (ADC) effectively and specifically targeted and depleted HSCs. A range of anti-CD45 PBD ADCs were designed and tested and it was determined that subsets of ADCs having PBD warheads had unexpectedly high efficacy when used for HSCT preconditioning.

As used herein, the term “anti-CD45 PBD ADC” refers to an ADC in which the antibody component is an anti-CD45 antibody and the drug component comprises a pyrrolobenzodiazepine (PBD), such as a PBD dimer. PBD dimers have been shown to form sequence selective, non-distorting and potently cytotoxic DNA interstrand cross-links in the minor groove of DNA. Typically therefore the PBD is able to bind to, and form interstrand cross-links in the minor groove of target cell DNA.

Accordingly, a first aspect of the present invention provides a conjugate of formula (I):

Ab-(DL)ₚ (I)

wherein:
Ab is an antibody that binds to CD45;
L is a linker connecting Ab to D;
D is a pyrrolobenzodiazepine (PBD), such as a PBD dimer;
and p is preferably from 1 to 8.

In some embodiments L may be absent, or simply a covalent bond between the antibody and the PBD.

In some embodiments the linker is a non-cleavable linker. As used herein, “non-cleavable linker” is used to refer to linkers that are not readily cleavable by enzyme activity, such as protease activity. An example of a non-cleavable drug-linker is drug-linker B4 described herein. The data presented herein indicate that the use of a non-cleavable linker can reduce the bystander effect. In some embodiments, a reduced bystander effect is desirable.

Non-cleavable linkers contrast to cleavable drug-linkers where the linker incorporates a sequence that is readily cleaved by enzyme action. For example, a known class of cleavable linkers incorporate a peptide sequence that can be readily cleaved by proteases such as cathepsin. An example of a cleavable drug-linker is drug-linker B1 described herein.

In preferred embodiments of the first aspect, the present invention provides a conjugate of formula (I):

Ab-(DL)ₚ (I)

wherein:
Ab is an antibody that binds to CD45;
DL is either:

### (a) DLa:

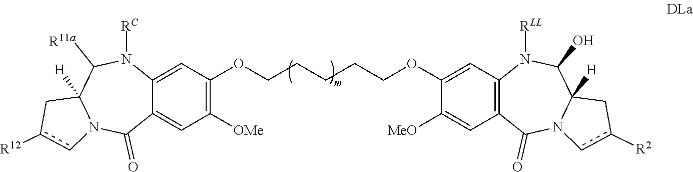

wherein:
R^{LL} is a linker for connection to Ab, which is

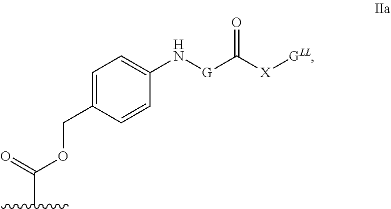

### Wherein

### Q is:

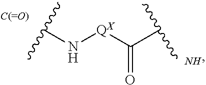

where Q^{X} is such that Q is an amino-acid residue, a dipeptide residue, a tripeptide residue or a tetrapeptide residue;

### X is:

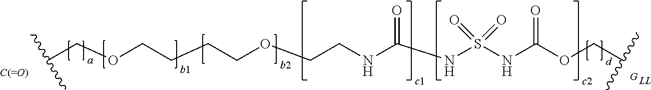

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0);
G^{LL} is a linker group connected to Ab;
either:
a) R^{11a} and R^{C} together form a double bond between the C and N atoms to which they are attached; or
b) R^{11a} is OH and R^{C} is:

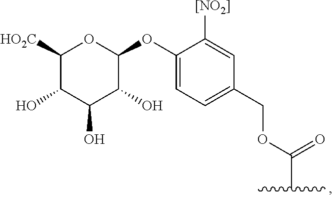

wherein the square brackets indicate the NO₂ group is optional;
m is 0 or 1;
when there is a double bond between C2 and C3, R² is methyl;
when there is a single bond between C2 and C3, R² is either H or

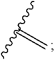

when there is a double bond between C2′ and C3′, R¹² is methyl;
when there is a single bond between C2′ and C3′, R¹² is H or

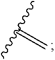

or

### (b) DLb:

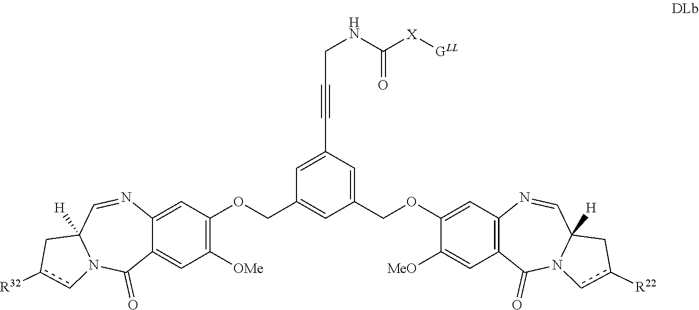

where X and G^{LL} are as defined above;
when there is a double bond between C2 and C3, R²² is methyl;
when there is a single bond between C2 and C3, R²² is either H or

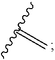

when there is a double bond between C2′ and C3′, R³² is methyl;
when there is a single bond between C2′ and C3′, R³² is H or

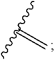

and
p is from 1 to 8.

The anti-CD45 PBD ADCs disclosed herein advantageously combine several features that allow for improvements to pre-condtioning therapies prior to donor cell engrafment or transplant.

Firstly, and as described elsewhere herein, following administration of the anti-CD45 PBD ADCs disclosed herein to a subject, the ADCs are preferably rapidly cleared from the subject's system in order to minimize the residual toxicity to any CD45+ve donor cells that are subsequently administered to the subject. This rapid clearance of the ADC may be achieved by a number of different mechanisms; for example, the antibody portion of the ADC may be selected, modified, or engineered so that it has a short half-life in the subject of interest. In a human subject, such an effect may be achieved by selecting a non-human antibody (e.g. a rodent antibody such as a rat antibody).

Despite their advantageously short residence time in the subject's system, the highly specific and potent PBD warheads of the anti-CD45 PBD ADCs disclosed herein allow for very high levels of lysis of the subjects HSCs, including—crucially—‘true’ HSCs. This cell population is thought to be CD45+CD34+CD38-Lin-CD45RA-CD90+ but with lower levels of expression of CD45 than more committed progenitors; with inadequate lysis levels resulting from existing targeted therapies believed to be a cause of sub-optimal transplant outcomes.

The drug loading is represented by p, the number of drug units per antibody. Drug loading may range from 1 to 8 Drug units (D) per antibody. For compositions, p represents the average drug loading of the conjugates in the composition, and p ranges from 1 to 8.

Also provided by the present disclosure are pharmaceutical compositions comprising the anti-CD45 PBD ADC disclosed herein. Further provided are methods of treating haematological cancer, methods of preparing a subject for transplantation of haematopoietic stem cells, and methods of engrafting stem cells in a subject, which utilise the anti-CD45 PBD ADC disclosed herein.

### BRIEF DESCRIPTION OF THE FIGURES

FIG. 1.
Expression levels of CD45 on human cell line and primary cells. (A) Expression levels of human CD45 purified CD34+ peripheral blood stem cells (PBSCs) and purified CD34+ cells from umbilical cord blood (UCB) from normal donors. (B) Northern blot analysis of CD45 RNA expression in various human tissues. PBMN, peripheral blood mononuclear cells.

FIG. 2.
Anti-human CD45 MAbs, YTH24.5 and YTH54.12, show synergistic lytic activity against CD45+ cells in complement dependent cytoxicity assays. Human CD45+ OCIM1 cells were radiolabelled with 51Cr and incubated with YTH24.5 alone, YTH54.12 alone or with both antibodies in presence of serum for two hours. Data are mean percentage of maximum lysis±s.d. of triplicate wells.

FIG. 3.
Anti-human CD45 antibodies are lytic in presence of serum and prevent colony formation in clonogenic assays. Clonogenic assays were carried out using human CD34+ cells from a normal donor treated with 10 ug/mL rat IgG2b isotype control or a mixture of 10 ug/mL of each anti-human CD45 MAb (YTH24.5+ YTH54.12) in (A) absence or (B) presence of 10% baby rabbit serum for 2 hours before plating into cytokine-supplemented semi-solid medium. Representative data is shown. Data are mean colony numbers±s.d. of triplicate wells. Representative images of colonies stained with p-iodonitro tetrazolium violet (INT) to visualise colonies are shown in the lower panel.

FIG. 4.
Ex vivo treatment of human CD34+ cells with anti-human CD45 MAbs in presence of serum prevents engraftment of human CD34+ cells in immunodeficient NSG mice. (A) Schematic overview of experimental protocol. CD34+ cells were cultured with 10 ug/mL rat IgG2b isotype antibody or a mixture of 10 ug/ml of each anti-human CD45 MAb (YTH24.5 and YTH54.12) in the presence of 10% rabbit serum for two hours before transplant into sublethally irradiated NSG mice. Mice were culled at 15 to 16 weeks post-transplant and haemopoietic tissues were analysed for human cell engraftment using flow cytometry. (B) Summarised data from two independent experiments at 15 and 16 weeks post-transplant showing engraftment levels of human cells (hCD45+) in the bone marrow (BM) of NSG mice. Data were analysed by Mann-Witney test.

FIG. 5.
Specific killing of human CD45+ cells by anti-human CD45-mediated internalization of saporin. Rat IgG2b isotype control, YTH24.5 and YTH54.12 MAbs alone or complexed with anti-rat IgG-saporin, were cultured with CD45+ Jurkat cells for 72 h hours. Data points are mean±s.d.

FIG. 6.
Anti-human CD45-PBDs specifically kill human CD45+ cell lines (rat IgG2b monoclonal antibody format; rlgG2b). Human CD45+ OCIM1 and Jurkat cell lines and human CD45-293T cells were cultured for 72 h hours in presence of Mab or ADC in triplicate wells. Cell viability was determined using PrestoBlue cell viability reagent. Data points are mean±s.d. Representative data from at least three experiments are shown.

FIG. 7.
Anti-human CD45-PBDs specifically kill human CD45+ cell lines (human IgG1-AAA monoclonal antibody format; hIgG1-AAA). Human CD45+ OCIM1 and Jurkat cell lines and human CD45-293T cells were cultured for 72 h hours in presence of Mab or ADC in triplicate wells. Cell viability was determined using PrestoBlue cell viability reagent. Data points are mean±s.d. Representative data from at least three experiments are presented.

FIG. 8.
Anti-CD45-PBDs kill human CD34+ clonogenic progenitors in clonogenic assays. Clonogenic assays were carried out using human CD34+ cells from a normal donor treated with MAb or ADC for 2 hours prior to plating into cytokine-supplemented semi-solid medium in triplicate wells. Colonies were scored after 10-14 days and expressed relative to untreated wells. Data are the means for three different donors±s.d.

FIG. 9.
Ex vivo treatment of human CD34+ cells with unconjugated anti-CD45 MAbs and CD45 PBDs prevent engraftment in NSG mice. Normal human CD34+ cells were cultured for 4 hours in presence of Mab alone or ADC at doses which ablated colony formation in clonogenic assays before transplantation into sublethally irradiated NSG mice. Analysis of human CD45+ cells in bone marrow at week 8 post-transplant by flow cytometry. Median values plotted.

FIG. 10.
Single dose of 3 mg/kg anti-human CD45-PBDs non-specifically reduce bone marrow cell numbers in humanized NSG mice with low levels of human cell engraftment. Sublethally irradiated NSG mice were transplanted with normal human CD34+ cells with low levels of human engraftment detected in the blood at 12 weeks post-transplant. Mice were treated with Mab or ADC at 3 mg/kg or 0.3 mg/kg and cellularity in the bone marrow was determined at 2 weeks post Mab/ADC injection. Data are mean±SEM for each treatment cohort.

FIG. 11.
PK studies. Non-humanized NSG mice were injected with 1 mg/kg of anti-human CD45 ADC and serial blood sampling was carried out. ELISA was used to determine the level of antibody in the serum. (A) YTH24.5-rlgG2b-PBD, 1 mg/kg, [T %=1.49 days]; (B) YTH54.12-rlgG2b-PBD, 1 mg/kg, [T %=1.53 days].

FIG. 12.
Anti-human CD45-PBDs deplete human CD45+ cells in the blood of humanised NSG mice. Sublethally irradiated NSG mice were transplanted with normal human CD34+ PBSCs. At 8 weeks post-transplant, mice were treated with 0.3 mg/kg or 1 mg/kg Mab or ADC. The levels of human CD45+ levels in the blood were determined over a two week period using flow cytometry. (A) Human CD45+ levels in the humanised treated with PBS or Mab alone at 0.3 mg/kg and 1 mg/kg. (B) Human CD45+ levels in the humanised treated with anti-human CD45 ADC at 0.3 mg/kg and 1 mg/kg.

FIG. 13.
Anti-CD45-PBDs deplete human CD45+ cells in the blood, BM and spleen of humanized NSG mice. Humanised NSG mice were treated at 8 weeks post-transplant with 0.3 mg/kg or 1 mg/kg Mab or ADC. Human CD45+ levels were determined in the blood, bone marrow spleen by flow cytometry after two weeks. Data are mean±SEM.

FIG. 14.
Anti-CD45 PBDs deplete human haematopoietic progenitor cells in the bone marrow of humanized NSG mice. Humanised NSG mice were treated at 8 weeks post-transplant with 0.3 mg/kg or 1 mg/kg Mab or ADC. Flow cytometry analysis of bone marrow was carried out at 2 weeks post-treatment and the number of human CD45+, human CD34+ and human CD34+/CD38-cells were determined. Data are mean±SEM.

FIG. 15.
Anti-CD45-PBDs completely delete human HSCs and MPPs in the bone marrow of humanised NSG mice. Flow cytometry analysis of bone marrow was carried out at 2 weeks post-treatment of humanised NSG mice. The number of immunophenotypic HSCs, MPPs and MLs were determined. Data are mean±SEM.

FIG. 16.
Anti-CD45-PBDs enhance the engraftment of GFP+ autologous CD34+ cells in the bone marrow of humanised NSG mice. Approximate protocol timeline.

FIG. 17.
Anti-CD45-PBDs enhance the engraftment of GFP+ autologous CD34+ cells in the bone marrow of humanised NSG mice. Sublethally irradiated NSG mice were transplanted with normal human CD34+ PBSCs. At 8 weeks post-transplant, mice were treated with 1 mg/kg anti-human CD45 Mab or ADC. At two weeks post-conditioning, mice received a transplant of GFP+ human PBSCs from the same human donor as the first transplant. Flow cytometry was carried out on the bone marrow after 9 weeks. (A) Percentage GFP+ human CD45+ cells of total human CD45+ cells in the bone marrow. (B) Percentage GFP+ human CD34+ cells of total human CD34+ cells in the bone marrow. (C) Percentage GFP+ human HSCs of total human HSC in the bone marrow. HSCs defined as CD34+/CD38−/CD45RA−/CD90+. (D) Percentage GFP+ human MLPs of total human MLPs in the bone marrow. MLPs defined as CD34+/CD38−/CD45RA+/CD90−. (E) Percentage GFP+ human MPPs of total human MPPs cells in the bone marrow. MPPs defined as CD34+/CD38−/CD45RA−/CD90−. The different treatment groups were compared with the Isotype ADC group using ordinary one-way ANOVA. Statistically significant differences between treatment groups are indicated by asterisks. Irradiation control group is included for reference only.

FIG. 18.
Anti-CD45-PBDs delay onset of leukaemia and prolong survival in NSG mice. Fire luciferase expressing human CD45+ Jurkat cells were transplanted into sublethally irradiated NSG mice and were monitored over a period of 70 days by bioluminescent imagining and for signs of morbidity. (A) Mean bioluminescent signal for each treatment group up to day 16. (B) Survival curve at day 70.

FIG. 19.
Higher expression levels of CD45 compared to c-Kit on human CD34+ cells. CD34 is a broader marker of haematopoietic lineage cells. Antigen density per cell is represented by the average number of molecules of each antigen determined using QuantiBRITE PE beads. Levels of c-Kit (left) are shown for the human AML cell line, OCIM1, bulk CD34+ cells and immunophenotypically defined HSCs, multipotent progenitors (MPPs) and multilymphoid progenitors (MLPs) from normal donors of peripheral blood stem cells. Levels of CD45 (right) on peripheral blood mononuclear cells (PBMCs), T cells, bulk CD34+ cells, HSCs, MPPs and MLPs.

FIG. 20.
Anti-human CD45 antibodies conjugated to B1, B4, B2 and B7 specifically kill human CD45+ cell lines. Cell viability of human CD45+ OCIM1 and Jurkat cell lines and human CD45-Nalm6 and 293T cells after 5 days in presence of Mab, ADC, or media alone. Comparison of YTH24.5, YTH54.12 and Isotype conjugated to B1 or B4 on A) OCIM1 cells, B) Jurkat, C) Nalm6 and D) HEK293T. Comparison of YTH24.5, YTH54.12 and Isotype rat IgG2b antibodies conjugated to B2 and B7 and unconjugated antibodies on E) OCIM1, F) Jurkat, G) Nalm6 and H) HEK293T. Data points are mean±s.d. Representative data is shown.

FIG. 21.
Anti-human CD45 antibodies, YTH24.5 and YTH54.12, conjugated to B1, B4, B2 and B7 specifically inhibit colony formation in clonogenic assays. Clonogenic assays performed using A) B1 conjugated or unconjugated antibodies, B) B4 conjugated or unconjugated antibodies, C) B2 conjugated or unconjugated antibodies and D) B7 conjugated or unconjugated antibodies. Pooled data points for three donors are shown (mean±s.d.).

FIG. 22.
Anti-human CD45 antibodies conjugated to B1, B4, B2 and B7 delay leukemia development and prolong survival in a NSG model of AML. Average bioluminescence signals±s.d. for each cohort of 5 mice treated with A) B1 conjugated YTH24.5, YTH54.12 and Isotype or unconjugated antibodies, B) B4 conjugated YTH24.5 and Isotype or unconjugated antibodies, C) B2 conjugated YTH24.5 and Isotype or unconjugated antibodies and D) B7 conjugated YTH24.5 and Isotype or unconjugated antibodies. E) Survival curve for all cohorts treated with the different ADCs.

FIG. 23.
Anti-human CD45 antibodies conjugated to B1, B4 and B2 induce tumor regression, delay in leukemia development and prolonged survival in NSG mice with established AML. A) Average bioluminescence signals±s.d. for each cohort of 5 mice treated with the unconjugated or conjugated antibodies. B) Survival curve for all cohorts treated with the different ADCs.

FIG. 24.
FIG. 5. Conditioning of humanized mice with YTH24.5-B1 enhanced engraftment of allogeneic human GFP+CD34+ cells. Mice humanized with CD34+ cells from one human donor were treated with either PBS or YTH24.5-B1 before transplant of GFP+CD34+ cells from another human donor. At 8 weeks after transplant of GFP+ cells, mice were culled the bone marrow analyzed by flow cytometry for A) total human CD45+ cell engraftment, B) % GFP+ human Cd45+ cells of all human Cd45+ cells and C) % GFP+ human CD34+ of all human CD34+ cells. Data points are mean±s.d. Unpaired t-test analysis was performed. Irradiation control was not included in statistical analysis.

FIG. 25.
FIG. 6. Comparison of anti-CD45 antibodies conjugated to different PBD payloads in an allogeneic stem cell transplant model in NSG mice. Mice humanized with CD34+ cells from one human donor were treated with either PBS or Isotype YTH24.5 (or YTH54.12) conjugated to three different PBD payloads before transplant of GFP+CD34+ cells from another human donor. At 8 weeks after transplant of GFP+ cells, mice were culled the bone marrow analyzed by flow cytometry for A) total human CD45+ cell engraftment, B) % GFP+ human CD45+ cells of all human CD45+ cells and C) % GFP+ human CD34+ of all human CD34+ cells. Data points are mean±s.d. Unpaired t-test analysis was carried out between Isotype ADC vs anti-CD45 ADC. Irradiation control was not included in statistical analysis.

### DETAILED DESCRIPTION OF THE INVENTION

### Background and Clinical Need

Haemopoietic Stem Cell Transplantation (HSCT) is curative for subjects with a wide range of malignant (including acute myeloid and acute lymphoblastic leukaemia, Non-Hodgkin's lymphoma, myeloma) and non-malignant (e.g. haemoglobinopathies and bone marrow failure) haematological disorders as well as genetic diseases (e.g. primary immunodeficiency [PID] and metabolic diseases). For subjects with genetic disorders of the haemopoietic system in whom no HLA-matched donor is available, gene therapy with autologous haemopoietic stem cells (HSCs) virally transduced with a corrected transgene are increasingly used.

However, for both HSCT and gene therapy, intensive pre-conditioning is necessary to achieve the myeloablation required to eradicate the subject's own HSCs and create a niche for the incoming graft. In addition, in HSCT immunosuppression is also used to prevent rejection and enable engraftment of donor HSCs.

The most commonly used conditioning methods are not targeted to the subjects own HSCs. For example, irradiation (such as total body irradiation) and DNA alkylating/modifying agents, are highly toxic to multiple organ systems, hematopoietic and non-hematopoietic cells, and the hematopoietic microenvironment. These harsh conditioning regimens effectively kill the host subject's immune and niche cells and adversely affect multiple organ systems, frequently leading to life-threatening complications such as veno-occlusive disease of the liver, gut mucositis and pneumonitis. Likewise, late adverse effects attributable to chemo/radiotherapy are common. These undesirable effects include growth retardation, infertility, cardiotoxicity and secondary malignancy.

These toxicities arise because conventional chemo/radiotherapy targets any dividing cell, not just the subject's own HSCs. To realize fully the curative potential of HSCT, the development of mild-conditioning regimens that avoid undesirable toxicity is essential. Needed are novel, preferably non-myeloablative, compositions and methods that may be used to condition a subject's tissues (e.g. bone marrow tissues), while lessening undesirable toxicity and minimizing the incidence of serious adverse reactions. Also needed are novel therapies that can selectively ablate an endogenous hematopoietic stem cell population in a target tissue, while minimizing or eliminating the effects of such therapies on non-targeted cells and tissues, such as platelets, white blood cells and red blood cells.

Cytolytic monoclonal antibodies (MAbs) provide an alternative means of achieving myelosuppression/immunosuppression without the non-haematological toxicity of chemotherapy, as the specificity of antibody binding offers the possibility of all but excluding ‘off-target’ toxicity. The successful development of this approach allows for hugely improved outcomes for subjects and greatly broadens the applicability of SCT and gene therapy.

### Targets for Antibody-Based Conditioning

Numerous candidate target molecules discussed in the literature, including CD13, CD33, CD34, CD44, CD45, CD49d: VLA-4, CD49f: VLA-6, CD59, CD84: CD150 family, CD90: Thyl, CD93, CD105: Endoglin, CD117: cKit/SCF receptor, CD123: IL-3R, CD126: IL-6R, CD133, CD135: Flt3 receptor, CD166: ALCAM, CD184: CXCR4, Prominin 2, Erythropoietin R, Endothelial Cell—Selective Adhesion Molecule, CD244, TieI, Tie2, MPL, G-CSFR or CSF3R, IL-1R, gp130, Leukemia inhibitory factor Receptor, oncostatin M receptor, Embigin and IL-18R [see, for example, WO/2016/164502//Abadir et al. 2019, Bone Marrow Transplantation; https://doi.org.//10.1038/s41409-019-0445-0//Czechowicz et al. 2019, Nature Communications; https://doi.org/10.1038/s41467-018-08201-x//WO1995/013093].

As a result of their research described in part herein, the present authors selected CD45 as their lead candidate.

Previously, c-kit had been considered as a potential target on haematopoietic stem cells (HSCs) for targeted conditioning agents. However, the present inventors have performed a number of key experiments described here that indicate that CD45 is superior as a HSC-specific target compared to c-Kit.

Substantially greater CD45 expression was observed on key cell types, as compared to c-Kit expression. c-Kit was previously a candidate target for a non-toxic conditioning product because of its enormous specificity for HSCs. However, HSC surface expression levels of CD45 were shown to be substantially higher than those for c-Kit (see FIG. 19). Much higher levels of CD45 protein than c-Kit receptors were observed across a range of peripheral and umbilical cord stem cell types, which might explain the superiority of the anti-CD45 combination to approaches targeting c-Kit.

CD45 is expressed on CD34+ haemopoietic progenitors at high density (FIG. 1A), but is absent on non-haemopoietic tissues (FIG. 1B). In rodent models, lytic anti-CD45 MAbs can induce aplasia [Dahlke, M. H., et al. (2002), Blood 99(10): 3566-3572.] and, whilst not sufficient to allow engraftment in immunocompetent mice, were able facilitate engraftment of allogeneic stem cells after non-myeloablative conditioning [Wulf, G. G., et al. (2003), Blood 101(6): 2434-2439.]. It is shown in FIG. 2 herein that the rat IgG2b anti-human CD45 MAbs, YTH 24.5 and YTH54.12, show synergistic lytic activity through complement in CD45+ cell lines.

The combination of these MAbs completely abrogates the ability of human CD34+ selected progenitors to give rise to myeloid and erythroid colonies in methylcellulose assays in the presence of complement, demonstrating that these antibodies effectively kill haemopoietic progenitors (FIG. 3). Moreover, ex vivo treatment of human CD34+ peripheral blood stem cells with a combination of YTH 24.5 and YTH54.12 in the presence of complement completely prevents engraftment of human haemopoiesis across all lineages in NSG mice, demonstrating that these antibodies are able to target human HSCs (FIG. 4).

### YTH24.5 and YTH54.12 Anti-CD45 MAbs in the Clinic

MAbs YTH24.5 and YTH54.12 are rapidly cleared from the circulation by virtue of their rat IgG2b Fc, resulting in a half-life of less that 12 hrs, allowing safe transplantation of the donor/gene corrected HSCs 2 days after the final antibody treatment [Krance, R. A., et al. (2003), Biol Blood Marrow Transplant 9(4): 273-281.3].

In a clinical study, the rapidly cleared, lytic anti-CD45 MAbs YTH24.5 and YTH54.12 were used for myelosuppression together with Alemtuzumab (an anti-CD52 MAb) for immunosuppression in a minimal-intensity conditioning regimen. It was demonstrated that this was sufficient to achieve curative engraftment in 13/16 paediatric subjects with PID with [Straathof, K. C., et al. (2009), Lancet 374(9693): 912-920.4]. Importantly, apart from manageable allergic reactions, no significant toxicity was observed to non-haemopoietic tissues and these subjects had no late effects related to conditioning.

However, the Staathof clinical study ibid. showed that myeloid engraftment was sub-optimal in some subjects and this may preclude extension of the existing anti-CD45-based approach to adults and subjects without underlying immunodeficiency analogous to that observed in rodent studies. Without being bound to any particular theory, the sub-optimal engraftment may be caused by inadequate lysis of true HSC (which are thought to be CD45+CD34+CD38-Lin-CD45RA-CD90+ but may have lower levels of expression of CD45 than more committed progenitors) so that an insufficient niche was created for donor HSC engraftment. Accordingly, the preseat autors looked to address this aspect by increasing the potency of targeted treatment against human HSCs.

### Rationale for Use of Anti-CD45 ADCs as Conditioning Agents

The present authors reasoned that the potency of the conditioning agents could be increased by developing naked anti-CD45 antibody conditioning agents into antibody-drug conjugates.

Several other ADC approaches have been reported in the literature. Czechowicz et al. 2019 ibid. reported selective and effective (>99% reported) depletion of host HSCs using an anti-CD177-saporin ADC. Palchaudhuri et al have developed a saporin-based immunotoxin targeting murine CD45 which depleted murine HSCs, enabled durable multi-lineage engraftment of congenic HSC in immunocompetent mice, and enabled engraftment of gene corrected murine HSCs in mice with sickle cell disease with minimal toxicity to non-haemopoietic tissues [Palchaudhuri, R., et al. (2016), Nat Biotechnol 34(7): 738-745//U.S. Ser. No. 10/280,225B2]. However, Palchaudhuri used anti-murine CD45 MAbs that do not target human HSCs. WO2020/146432 exemplifies the use of an anti-CD45 ADC having an amanatin warhead to promote acceptance of CAR-T cell therapy.

Furthermore, it has been confirmed using live-imaging microscopy that rat IgG2b YTH24.5 and YTH54.12 are substantially internalised via a lysosomal pathway (data not shown). The fact that the antibodies are readily internalised by the target cells supports the hypothesis that these antibodies are suited to an ADC-based approach. In particular, the lysosomal pathway can be important in providing an environment for cleavage of the antibody-drug linkers, hereby releasing the drug in the target cells for highly specific cell killing.

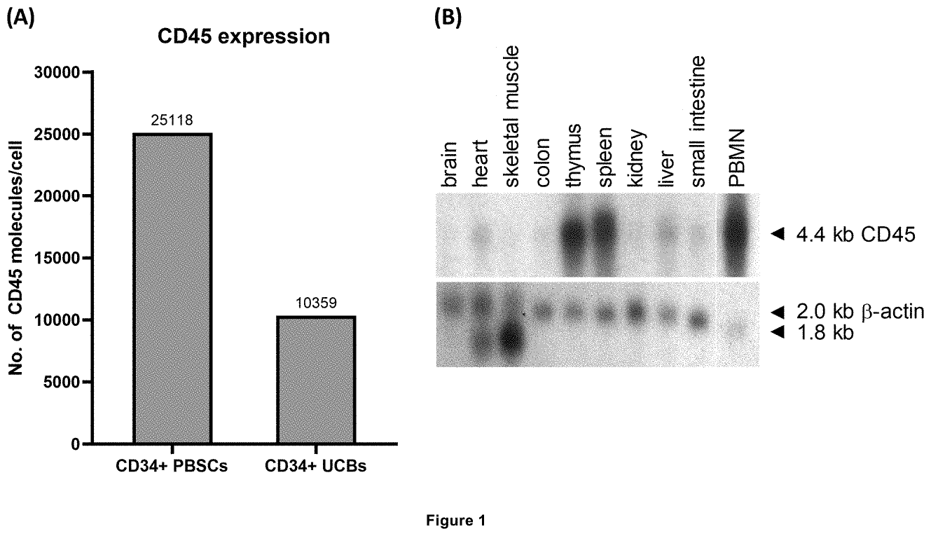

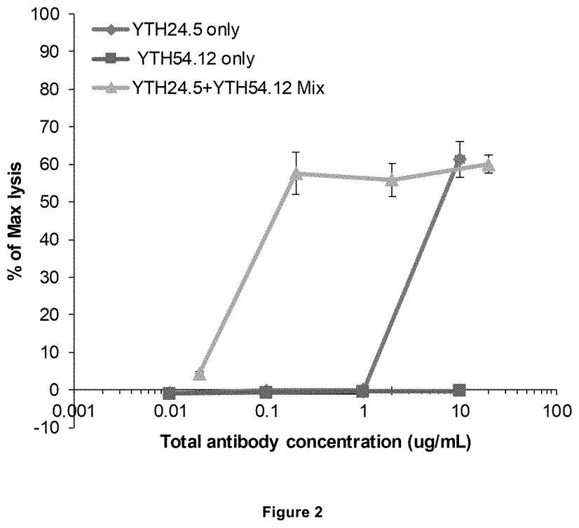

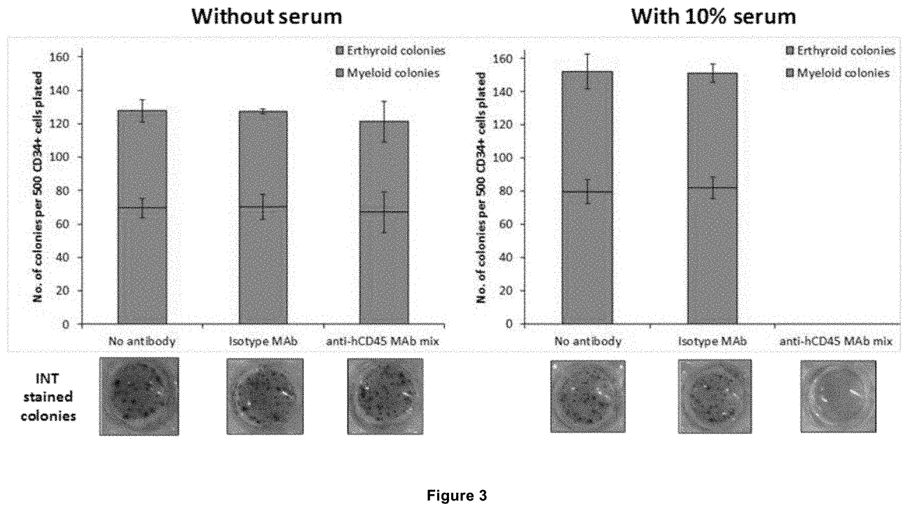

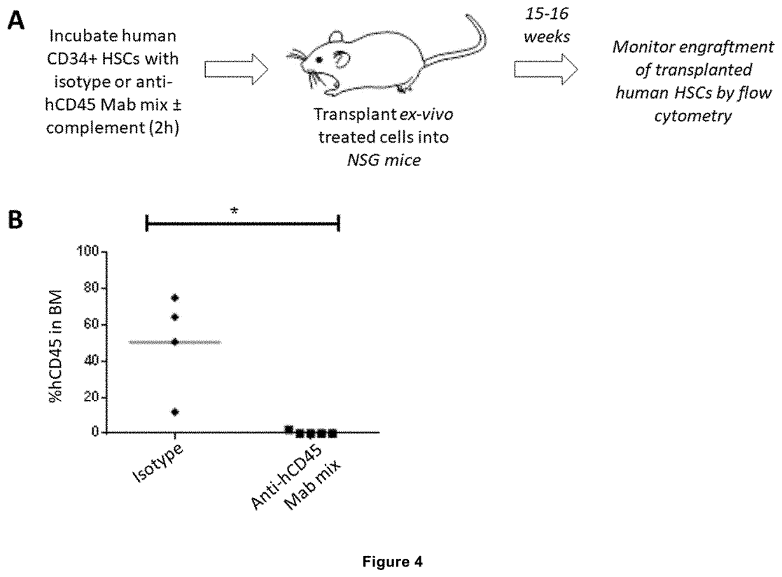

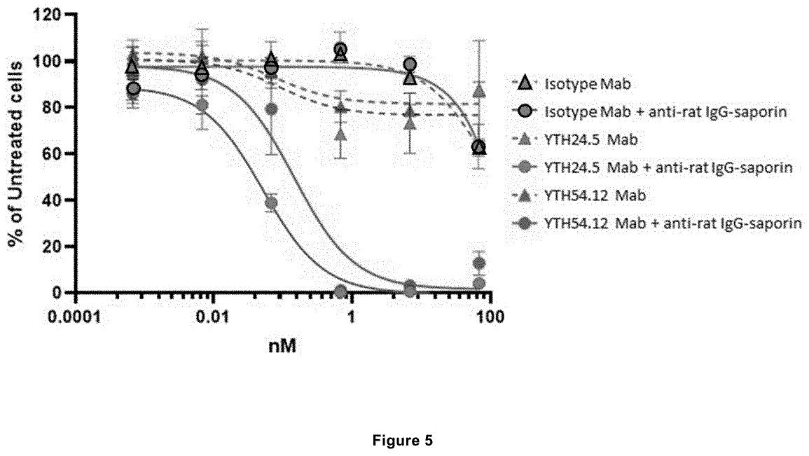

As proof of principle, the present authors have shown that MAbs YTH24.5 and YTH54.12 complexed with anti-rat IgG saporin are able to mediate killing of human CD45+ cell lines at subnanomolar levels, demonstrating that anti-CD45 ADCs can be internalised by human cells (FIG. 5).

In their next phase of research, the present authors sought to achieve further improvements by exchanging the saporin payload for other cytotoxic molecules such as saporin, Monomethyl auristatin E (MMAE), an anthracycline, amanitin, duocarmycin, calicheamycin, Pseudomonas exotoxin A, alpha sarcin, and camptothecin. Through this work it was unexpectedly found that a subset of PBD payloads had unexpectedly high efficacy when used for HSCT preconditioning.

### Anti-CD45 PBD ADCs

As used herein, the term “anti-CD45 PBD ADC” refers to an antibody drug conjugate (ADC) in which the antibody component is an anti-CD45 antibody and the drug component comprises a pyrrolobenzodiazepine (PBD), such as a PBD dimer. PBD dimers have been shown to form sequence selective, non-distorting and potently cytotoxic DNA interstrand cross-links in the minor groove of DNA. Typically therefore the PBD is able to bind to, and form interstrand cross-links in the minor groove of target cell DNA.

Accordingly, the present disclosure provides ADCs comprising an anti-CD45 antibody conjugated to a PBD payload.

In some embodiments (where DL=DLa), the present disclosure provides a PBD dimer with a linker connected through the N10 position on one of the PBD moieties conjugated to an antibody as defined below, with an optional capping on the non-linked N10 position.

These embodiments are suitable for use in providing a PBD compound to a preferred site in a subject. The conjugate allows the release of an active PBD compound that does not retain any part of the linker. There is no stub present that could affect the reactivity of the PBD compound. Thus the conjugate of formula (I) could release the compound RelA:

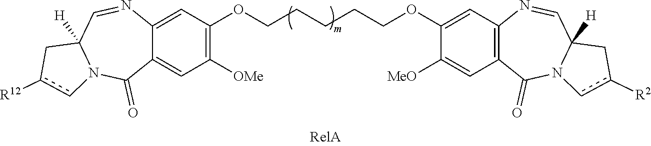

The specified link between the PBD dimer and the antibody in the present disclosure is preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Delivery of the compounds of, for example, formulae RelA is achieved at the desired activation site of the conjugate of formula (I) by the action of an enzyme, such as cathepsin, on the linking group, and in particular on the peptide moiety.

In some embodiments (where DL=DLb), the drug-linker is released by antibody degradation. Similar considerations as discussed above about the linker stability are applicable to these embodiments, although there is no specific group in the linker moiety which is susceptible to cleavage.

### Antibody

In one aspect the antibody is an antibody that binds to CD45.

CD45 is also known as Protein tyrosine phosphatase, receptor type, C (PTPRC). The protein is a type-I Transmembrane protein that has protein tyrosine phosphatase activity and is expressed in all nucleated cells of hematopoietic origin, often at a high level (5-10% of membrane protein). CD45 interacts with numerous immune cell proteins; for example, it has been demonstrated to be an essential regulator of both T-cell and B-cell antigen receptor signalling. Transgenic mice lacking CD45 have SCID (Yoo et al., 2000) whereas mice with an activiating CD45 mutation exhibit lymphoproliferation, autoantibody production, and severe nephritis (Majeti et al., 2000).

For the purposes of the present disclosure, CD45 represents an attractive target for an antibody-drug conjugate (ADC) approach as it is selectively expressed on all leucocytes and haemopoietic progenitors, but is absent on non-haemopoietic tissues (Straathof K et al. 2009, DOI:10.1016/S0140-6736(09)60945-4).

The CDRs of antibody variable domains described herein may be identified by any suitable method known in the art, for example using any suitable antibody numbering scheme. The CDRs may be identified using any of the Kabat numbering scheme (Kabat et al., U.S. Department of Health and Human Services, 1991), the Chothia numbering scheme (Chothia C, Lesk A M. J Mol Biol. (1987) 196:901-17), or the IMGT numbering scheme (Giudicelli V, et al. Nucleic Acids Res. (1997) 25:206-11; Lefranc M P. Immunol Today (1997) 18:509). The skilled person will appreciate that these different CDR labelling systems can give slightly different results, but in each case the CDRs can be easily identified by the skilled person.

The CDR sequences as disclosed herein have been identified and defined using the Kabat numbering scheme (Kabat et al., U.S. Department of Health and Human Services, 1991).

### YTH 24.5

In some embodiments the antibody comprises a VH domain having a VH CDR3 with the amino acid sequence of SEQ ID NO. 5. In some embodiments the VH domain further comprises a VH CDR2 with the amino acid sequence of SEQ ID NO. 4, and/or a VH CDR1 with the amino acid sequence of SEQ ID NO. 3. In some embodiments the the antibody comprises a VH domain having a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5. In some embodiments the antibody a VH domain comprising a VH CDR1, a VH CDR2, and a VH CDR3, wherein the antibody comprises the CDR sequences of the VH domain having the sequence of SEQ ID NO: 1. In preferred embodiments the antibody comprises a VH domain having the sequence according to SEQ ID NO. 1.

The antibody may further comprise a VL domain. In some embodiments the antibody comprises a VL domain having a VL CDR3 with the amino acid sequence of SEQ ID NO. 8. In some embodiments the VL domain further comprises a VL CDR2 with the amino acid sequence of SEQ ID NO. 7, and/or a VL CDR1 with the amino acid sequence of SEQ ID NO. 6. In some embodiments the the antibody comprises a VL domain having a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8. In some embodiments the antibody a VL domain comprising a VL CDR1, a VL CDR2, and a VL CDR3, wherein the antibody comprises the CDR sequences of the VL domain having the sequence of SEQ ID NO: 2. In preferred embodiments the antibody comprises a VL domain having the sequence according to SEQ ID NO. 2.

In preferred embodiments the antibody comprises a VH domain and a VL domain. Preferably the VH comprises the sequence of SEQ ID NO. 1 and the VL domain comprises the sequence of SEQ ID NO. 2.

The VH and VL domain(s) may form an antibody antigen binding site that binds CD45.

In some embodiments the antibody is an intact antibody comprising a VH domain and a VL domain, the VH and VL domains having sequences of SEQ ID NO. 1 paired with SEQ ID NO. 2.

In some embodiments the antibody comprises a heavy chain having the sequence of SEQ ID NO. 9 paired with a light chain having the sequence of SEQ ID NO. 10. In some embodiments the antibody is an intact antibody comprising two heavy chains each having the sequence of SEQ ID NO. 9, and two light chains each having the sequence of SEQ ID NO. 10.

In some embodiments the antibody is the YTH 24.5 as disclosed in WO1995/013093.

### YTH 54.12

In some embodiments the antibody comprises a VH domain having a VH CDR3 with the amino acid sequence of SEQ ID NO. 15. In some embodiments the VH domain further comprises a VH CDR2 with the amino acid sequence of SEQ ID NO. 14, and/or a VH CDR1 with the amino acid sequence of SEQ ID NO. 13. In some embodiments the the antibody comprises a VH domain having a VH CDR1 with the amino acid sequence of SEQ ID NO. 13, a VH CDR2 with the amino acid sequence of SEQ ID NO. 14, and a VH CDR3 with the amino acid sequence of SEQ ID NO. 15. In some embodiments the antibody a VH domain comprising a VH CDR1, a VH CDR2, and a VH CDR3, wherein the antibody comprises the CDR sequences of the VH domain having the sequence of SEQ ID NO: 11. In preferred embodiments the antibody comprises a VH domain having the sequence according to SEQ ID NO. 11.

The antibody may further comprise a VL domain. In some embodiments the antibody comprises a VL domain having a VL CDR3 with the amino acid sequence of SEQ ID NO. 18. In some embodiments the VL domain further comprises a VL CDR2 with the amino acid sequence of SEQ ID NO. 17, and/or a VL CDR1 with the amino acid sequence of SEQ ID NO. 16. In some embodiments the the antibody comprises a VL domain having a VL CDR1 with the amino acid sequence of SEQ ID NO. 16, a VL CDR2 with the amino acid sequence of SEQ ID NO. 17, and a VL CDR3 with the amino acid sequence of SEQ ID NO. 18. In some embodiments the antibody a VL domain comprising a VL CDR1, a VL CDR2, and a VL CDR3, wherein the antibody comprises the CDR sequences of the VL domain having the sequence of SEQ ID NO: 12. In preferred embodiments the antibody comprises a VL domain having the sequence according to SEQ ID NO. 12.

In preferred embodiments the antibody comprises a VH domain and a VL domain. Preferably the VH comprises the sequence of SEQ ID NO. 11 and the VL domain comprises the sequence of SEQ ID NO. 12.

The VH and VL domain(s) may form an antibody antigen binding site that binds CD45.

In some embodiments the antibody is an intact antibody comprising a VH domain and a VL domain, the VH and VL domains having sequences of SEQ ID NO. 11 paired with SEQ ID NO. 12.

In some embodiments the antibody comprises a heavy chain having the sequence of SEQ ID NO. 19 paired with a light chain having the sequence of SEQ ID NO. 20. In some embodiments the antibody is an intact antibody comprising two heavy chains each having the sequence of SEQ ID NO. 19, and two light chains each having the sequence of SEQ ID NO. 20.

In some embodiments the antibody is the YTH 54.12 as disclosed in WO1995/013093.

### YTH 24.5/54.12

In some embodiments the antibody comprises a first antigen binding domain and a second antigen binding domain, wherein:
(i) the first antigen binding domain comprises a VH domain and, optionally, a VL domain as described herein above in the section entitled “YTH 24.5”; and
(ii) the second antigen binding domain comprises a VH domain and, optionally, a VL domain as described herein above in the section entitled “YTH 54.12”.

In one aspect the antibody is an antibody as described herein which has been modified (or further modified) as described below. In some embodiments the antibody is a humanised, deimmunised or resurfaced version of an antibody disclosed herein.

As described elsewhere herein, following administration of the anti-CD45 ADCs disclosed herein to a subject, the ADCs are preferably rapidly cleared from the subject's system in order to minimize the residual toxicity to any CD45+ve cells that may subsequently administered to the subject. This rapid clearance of the ADC may be achieved by a number of different mechanisms; for example, the antibody portion of the ADC may be selected, modified, or engineered so that it has a short half-life in the subject of interest. In a human subject, such an effect may be achieved by selecting a non-human antibody (e.g. a rat antibody).

Accordingly, in some cases the antibody or fragment thereof comprises a constant region, which may be a full-length constant region. The antibody can be of any isotype. The antibody may be, for example, IgA1, IgA2, IgD, IgE, IgG1, IgG2, IgG3, IgG4 or IgM. Preferably, the antibody may be an IgM or an IgG antibody. Most preferably, the antibody may be an IgG1, an IgG2 or an IgG3 antibody. In some cases the antibody is a full-length antibody. The antibody may be a non-human antibody. The antibody may be a murine rodent antibody, i.e., a rat or mouse antibody. The antibody may be a monkey antibody. In some cases the antibody is a rat IgG2 antibody, more preferably a rat IgG2b antibody. Such non-human antibodies, preferably rat antibodies, have the advantage that they result in rapid clearance of the antibody from the system of human subjects, as described above. In some cases where the antibody is a biparatopic or bispecific antibody as described herein, the antibody may be a human isotype hybrid antibody (such as for example, a human IgG2/IgG4 hybrid antibody) or a rat/mouse hybrid antibody (such as for example, a mouse IgG2a/rat IgG2b hybrid antibody).

### Terminology

The term “antibody” herein is used in the broadest sense and specifically covers monoclonal antibodies, polyclonal antibodies, dimers, multimers, multispecific antibodies (e.g., bispecific antibodies), intact antibodies and antibody fragments, so long as they exhibit the desired biological activity, for example, the ability to bind CD45. Antibodies may be murine, rat, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, rat, murine, or rabbit origin.

As used herein, “binds CD45” is used to mean the antibody binds CD45 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD45 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies disclosed herein can bind CD45 with a high affinity. For example, in some embodiments the antibody can bind CD45 with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

CD45 (also known as Protein tyrosine phosphatase, receptor type, C [PTPRC]) is a member of the protein tyrosine phosphatase family of signalling molecules. In some embodiments, the CD45 polypeptide corresponds to Genbank accession no. CAA68669, version no. CAA68669.1, record update date: Feb. 2, 2011 10:53 AM. In one embodiment, the nucleic acid encoding CD45 polypeptide corresponds to Genbank accession no. Y00638, version no Y00638.1, record update date: Feb. 2, 2011 10:53 AM. In some embodiments, the CD45 polypeptide is as described in UniProt record P08575-3. In some embodiments, the CD45 polypeptide has the sequence of SEQ ID NO. 21.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al. (1991) Nature, 352:624-628; Marks et al. (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

### Modification of Antibodies

The antibodies disclosed herein may be modified. For example, to make them less immunogenic to a human subject. This may be achieved using any of a number of techniques familiar to the person skilled in the art. Some of these techniques are described in more detail below.

### Humanisation

Techniques to reduce the in vivo immunogenicity of a non-human antibody or antibody fragment include those termed “humanisation”.

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies. The expression “humanized antibody” also includes an immunoglobulin amino acid sequence variant or fragment thereof that comprises an FR having substantially the amino acid sequence of a human immunoglobulin and a CDR having substantially the amino acid sequence of a non-human immunoglobulin.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g. murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins.

There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

### CDR Grafting

In this technique, the humanized antibodies are human immunoglobulins (recipient antibody) in which residues from a complementary-determining region (CDR) of the recipient antibody are replaced by residues from a CDR of a non-human species (donor antibody) such as mouse, rat, camel, bovine, goat, or rabbit having the desired properties (in effect, the non-human CDRs are ‘grafted’ onto the human framework). In some instances, framework region (FR) residues of the human immunoglobulin are replaced by corresponding non-human residues (this may happen when, for example, a particular FR residue has significant effect on antigen binding).

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin.

### Guided Selection

The method consists of combining the V_{H} or V_{L} domain of a given non-human antibody specific for a particular epitope with a human V_{H} or V_{L} library and specific human V domains are selected against the antigen of interest. This selected human VH is then combined with a VL library to generate a completely human VH×VL combination. The method is described in Nature Biotechnology (N.Y.) 12, (1994) 899-903.

### Composite Antibodies

In this method, two or more segments of amino acid sequence from a human antibody are combined within the final antibody molecule. They are constructed by combining multiple human VH and VL sequence segments in combinations which limit or avoid human T cell epitopes in the final composite antibody V regions. Where required, T cell epitopes are limited or avoided by, exchanging V region segments contributing to or encoding a T cell epitope with alternative segments which avoid T cell epitopes. This method is described in US 2008/0206239 A1.

### Deimmunization

This method involves the removal of human (or other second species) T-cell epitopes from the V regions of the therapeutic antibody (or other molecule). The therapeutic antibodies V-region sequence is analysed for the presence of MHC class II-binding motifs by, for example, comparison with databases of MHC-binding motifs (such as the “motifs” database hosted at www.wehi.edu.au). Alternatively, MHC class II-binding motifs may be identified using computational threading methods such as those devised by Altuvia et al. (J. Mol. Biol. 249 244-250 (1995)); in these methods, consecutive overlapping peptides from the V-region sequences are testing for their binding energies to MHC class II proteins. This data can then be combined with information on other sequence features which relate to successfully presented peptides, such as amphipathicity, Rothbard motifs, and cleavage sites for cathepsin B and other processing enzymes.

Once potential second species (e.g. human) T-cell epitopes have been identified, they are eliminated by the alteration of one or more amino acids. The modified amino acids are usually within the T-cell epitope itself, but may also be adjacent to the epitope in terms of the primary or secondary structure of the protein (and therefore, may not be adjacent in the primary structure). Most typically, the alteration is by way of substitution but, in some circumstances amino acid addition or deletion will be more appropriate.

All alterations can be accomplished by recombinant DNA technology, so that the final molecule may be prepared by expression from a recombinant host using well established methods such as Site Directed Mutagenesis. However, the use of protein chemistry or any other means of molecular alteration is also possible.

### Resurfacing

This method involves:
(a) determining the conformational structure of the variable region of the non-human (e.g. rodent) antibody (or fragment thereof) by constructing a three-dimensional model of the non-human antibody variable region;
(b) generating sequence alignments using relative accessibility distributions from x-ray crystallographic structures of a sufficient number of non-human and human antibody variable region heavy and light chains to give a set of heavy and light chain framework positions wherein the alignment positions are identical in 98% of the sufficient number of non-human antibody heavy and light chains;
(c) defining for the non-human antibody to be humanized, a set of heavy and light chain surface exposed amino acid residues using the set of framework positions generated in step (b);
(d) identifying from human antibody amino acid sequences a set of heavy and light chain surface exposed amino acid residues that is most closely identical to the set of surface exposed amino acid residues defined in step (c), wherein the heavy and light chain from the human antibody are or are not naturally paired;
(e) substituting, in the amino acid sequence of the non-human antibody to be humanized, the set of heavy and light chain surface exposed amino acid residues defined in step (c) with the set of heavy and light chain surface exposed amino acid residues identified in step (d);
(f) constructing a three-dimensional model of the variable region of the non-human antibody resulting from the substituting specified in step (e);
(g) identifying, by comparing the three-dimensional models constructed in steps (a) and (f), any amino acid residues from the sets identified in steps (c) or (d), that are within 5 Angstroms of any atom of any residue of the complementarity determining regions of the non-human antibody to be humanized; and
(h) changing any residues identified in step (g) from the human to the original non-human amino acid residue to thereby define a non-human antibody humanizing set of surface exposed amino acid residues; with the proviso that step (a) need not be conducted first, but must be conducted prior to step (g).

### Superhumanization

The method compares the non-human sequence with the functional human germline gene repertoire. Those human genes encoding canonical structures identical or closely related to the non-human sequences are selected. Those selected human genes with highest homology within the CDRs are chosen as FR donors. Finally, the non-human CDRs are grafted onto these human FRs. This method is described in patent WO 2005/079479 A2.

### Human String Content Optimization

This method compares the non-human (e.g. mouse) sequence with the repertoire of human germline genes and the differences are scored as Human String Content (HSC) that quantifies a sequence at the level of potential MHC/T-cell epitopes. The target sequence is then humanized by maximizing its HSC rather than using a global identity measure to generate multiple diverse humanized variants (described in Molecular Immunology, 44, (2007) 1986-1998).

### Framework Shuffling

The CDRs of the non-human antibody are fused in-frame to cDNA pools encompassing all known heavy and light chain human germline gene frameworks. Humanised antibodies are then selected by e.g. panning of the phage displayed antibody library. This is described in Methods 36, 43-60 (2005).

### EMBODIMENTS

### Q^{X}

In one embodiment, Q is an amino acid residue. The amino acid may be a natural amino acid or a non-natural amino acid.

In one embodiment, Q is selected from: Phe, Lys, Val, Ala, Cit, Leu, Ile, Arg, and Trp, where Cit is citrulline.

In one embodiment, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q is selected from:
^{C═O}-Phe-Lys-^{NH},
^{C═O}-Val-Ala-^{NH},
^{C═O}-Val-Lys-^{NH},
^{C═O}-Ala-Lys-^{NH},
^{C═O}-Val-Cit-^{NH},
^{C═O}-Phe-Cit-^{NH},
^{C═O}-Leu-Cit-^{NH},
^{C═O}Ile-Cit-^{NH},
^{C═O}-Phe-Arg-^{NH},
^{C═O}-Trp-Cit-^{NH}, and
^{C═O}-Gly-Val-^{NH};
   where Cit is citrulline.

Preferably, Q is selected from:
^{C═O}-Phe-Lys-^{NH},
^{C═O}-Val-Ala-^{NH},
^{C═O}-Val-Lys-^{NH},
^{C═O}-Ala-Lys-^{NH}, and
^{C═O}-Val-Cit-^{NH}.

Most preferably, Q is selected from ^{C═O}-Phe-Lys-^{NH}, ^{C═O}-Val-Cit-^{NH} or ^{C═O}-Val-Ala-^{NH}.

Other dipeptide combinations of interest include:
^{C═O}-Gly-Gly-^{NH}
^{C═O}-Gly-Val-^{NH}
^{C═O}Pro-Pro-^{NH}, and
^{C═O}-Val-Glu-^{NH}.

Other dipeptide combinations may be used, including those described by Dubowchik et al., Bioconjugate Chemistry, 2002, 13,855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{C═O}-Glu-Val-Ala-^{NH}
^{C═O}-Glu-Val-Cit-_{NH}
^{C═O}-αGlu-Val-Ala-_{NH}
^{C═O}-αGlu-Val-Cit-_{NH}

In some embodiments, Q is a tetrapeptide residue. The amino acids in the tetrapeptide may be any combination of natural amino acids and non-natural amino acids. In some embodiments, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:
^{C═O}-Gly-Gly-Phe-Gly-^{NH}; and
^{C═O}-Gly-Phe-Gly-Gly-^{NH}.

In some embodiments, the tetrapeptide is:
^{C═O}-Gly-Gly-Phe-Gly-^{NH}

In the above representations of peptide residues, ^{C═O}-represents where the residue binds to C═O in R^{LL}, and -^{NH} represents where the residue binds to NH in R^{LL}.

Glu represents the residue of glutamic acid, i.e.:

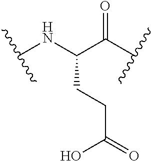 [SMILES: *N[C@@H](CCC(=O)O)C(*)=O]

αGlu represents the residue of glutamic acid when bound via the α-chain, i.e.:

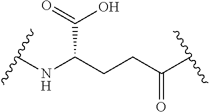 [SMILES: *N[C@@H](CCC(*)=O)C(=O)O]

In one embodiment, the amino acid side chain is chemically protected, where appropriate. The side chain protecting group may be a group as discussed above. Protected amino acid sequences are cleavable by enzymes, such as cathepsin. For example, a dipeptide sequence comprising a Boc side chain-protected Lys residue is cleavable by cathepsin.

Protecting groups for the side chains of amino acids are well known in the art and are described in the Novabiochem Catalog, and as described above.

### G^{LL}

G^{LL} may be selected from:

| | |
|---|---|
| 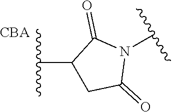 [SMILES: *C1CC(=O)N(*)C1=O] | (G^{LL1-1}) |
| 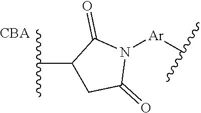 | (G^{LL1-2}) |
| 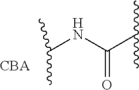 [SMILES: *NC(*)=O] | (G^{LL2}) |
| 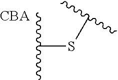 [SMILES: *S*] | (G^{LL3-1}) |
| 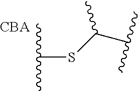 [SMILES: *SC(*)C] | (G^{LL3-2}) |
| 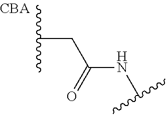 [SMILES: *CC(=O)N*] | (G^{LL-4}) |
| 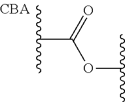 [SMILES: *OC(*)=O] | (G^{LL5}) |
| 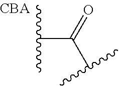 [SMILES: *C(*)=O] | (G^{LL6}) |
| 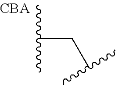 [SMILES: *C*] | (G^{LL7}) |
| 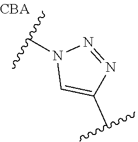 [SMILES: *c1cn(*)nn1] | (G^{LL8-1}) |
| 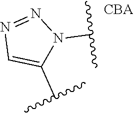 [SMILES: *c1cnnn1*] | (G^{LL8-2}) |
| 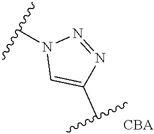 [SMILES: *c1cn(*)nn1] | (G^{LL9-1}) |
| 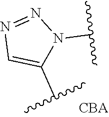 [SMILES: *c1cnnn1*] | (G^{LL9-2}) |
| 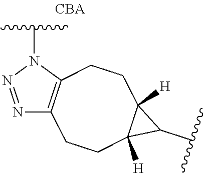 [SMILES: *C1[C@H]2CCc3c(nnn3*)CC[C@@H]12] | (G^{LL10}) |
| 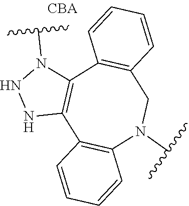 [SMILES: *N1NNC2=C1c1ccccc1CN(*)c1ccccc12] | (G^{LL11}) |
| 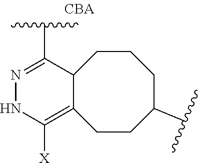 | (G^{LL12}) |
| 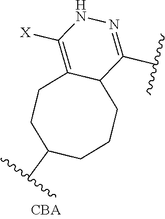 | (G^{LL13}) |
| 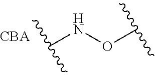 [SMILES: *NO*] | (G^{LL14}) |

where Ar represents a C5-6 arylene group, e.g. phenylene and X represents C₁₋₄ alkyl.

In some embodiments, G^{LL} is selected from G^{LL1-1} and G^{LL1-2}. In some of these embodiments, G^{LL} is G^{LL1-1}.

In some embodiments, G^{LL} is G^{LL10}.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C6);
N₂O₁: oxadiazole (furazan) (C5);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

### X

### X is:

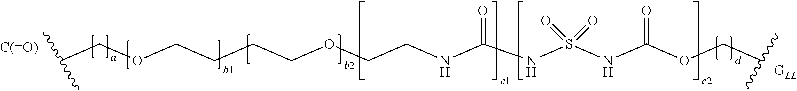

where a=0 to 5, b1=0 to 16, b2=0 to 16, c1=0 or 1, c2=0 or 1, d=0 to 5.
a may be 0, 1, 2, 3, 4 or 5. In some embodiments, a is 0 to 3. In some of these embodiments, a is 0 or 1. In further embodiments, a is 0. In further embodiments, a is 1.
b1 may be 0, 1, 2, 3, 4, 5, 6, 7, 8, 9, 10, 11, 12, 13, 14, 15 or 16. In some embodiments, b1 is 0 to 12. In some of these embodiments, b1 is 0 to 8, and may be 0, 2, 3, 4, 5 or 8. In further embodiments, b1 is 2.
b2 may be 0, 1, 2, 3, 4, 5, 6, 7, 8, 9, 10, 11, 12, 13, 14, 15 or 16. In some embodiments, b is 0 to 12. In some of these embodiments, b2 is 0 to 8, and may be 0, 2, 4, 5 or 8. In further embodiments, b2 is 8.

Only one of b1 and b2 may not be 0.

c1 may be 0 or 1.
c2 may be 0 or 1.

Only one of c1 and c2 may not be 0.

d may be 0, 1, 2, 3, 4 or 5. In some embodiments, d is 0 to 3. In some of these embodiments, d is 1 or 2. In further embodiments, d is 2. In further embodiments, d is 5.

In some embodiments of X, a is 0, b1 is 0, c1 is 1, c2 is 0 and d is 2, and b2 may be from 0 to 8. In some of these embodiments, b2 is 0, 4, 5 or 8. In further embodiments, b2 is 8.

In some embodiments of X, a is 1, b2 is 0, c1 is 0, c2 is 1, d is 2, and b1 may be from 0 to 8. In some of these embodiments, b1 is 2.

### Further Embodiments of a to d

In some further embodiments of X, a=0 to 5, b1=0, b2=0 to 16, c1=0 or 1, c2=0 and d=0 to 5.

In these further embodiments, a may be 0, 1, 2, 3, 4 or 5. In some of these further embodiments, a is 0 to 3. In some of these further embodiments, a is 0 or 1, and may be 0.

In these further embodiments, b2 may be 0, 1, 2, 3, 4, 5, 6, 7, 8, 9, 10, 11, 12, 13, 14, 15 or 16. In some of these further embodiments, b2 is 0 to 12. In some of these further embodiments, b2 is 0 to 8, and may be 0, 2, 4 or 8.

In these further embodiments, c1 may be 0 or 1.

In these further embodiments, d may be 0, 1, 2, 3, 4 or 5. In some of these further embodiments, d is 0 to 3. In some of these further embodiments, d is 1 or 2, and may be 2.

In some of these further embodiments of X, a is 0, c2 is 1 and d is 2, and b2 may be from 0 to 8. In some of these further embodiments, b2 is 0, 4 or 8.

m

In some embodiments, m is 0.

In some embodiments, m is 1.

R^{C} and R^{11a}

In some embodiments, R^{11a} and R^{C} together form a double bond between the C and N atoms to which they are attached.

In some embodiments, R^{11a} is OH and R^{C} is:

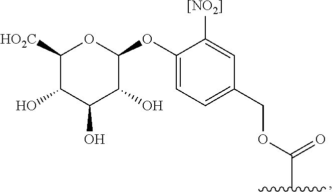

wherein the square brackets indicate the NO₂ group is optional. In some of these embodiments, the NO₂ group is present.
R² and R¹²

In some embodiments, R² and R¹² are the same.

In some embodiments, there is a double bond between C2 and C3 and between C2′ and C3, and R² and R¹² are both methyl.

In some embodiments, there is a single bond between C2 and C3 and between C2′ and C3, and R² and R¹² are both H.

In some embodiments, there is a single bond between C2 and C3 and between C2′ and C3, and R² and R¹² are both

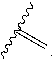

R²² and R³²

In some embodiments, R²² and R³² are the same.

In some embodiments, there is a double bond between C2 and C3 and between C2′ and C3, and R²² and R³² are both methyl.

In some embodiments, there is a single bond between C2 and C3 and between C2′ and C3, and R²² and R³² are both H.

In some embodiments, there is a single bond between C2 and C3 and between C2′ and C3, and R²² and R³² are both

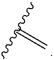

### DL

In some embodiments of the disclosure. DL is selected from:

| | |
|---|---|
| 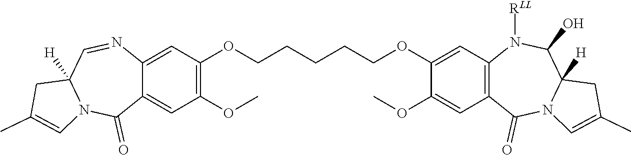 | A1 |
| 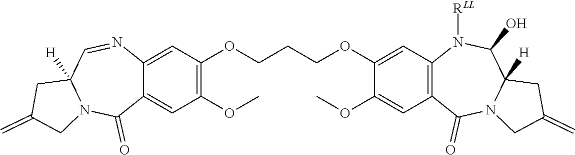 | A2 |
| 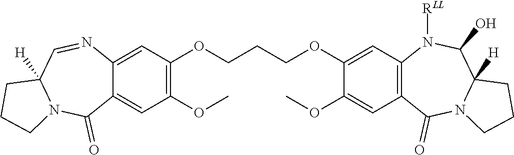 | A3 |
| 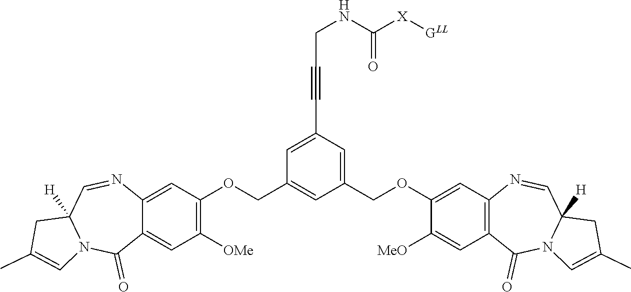 | A4 |
| 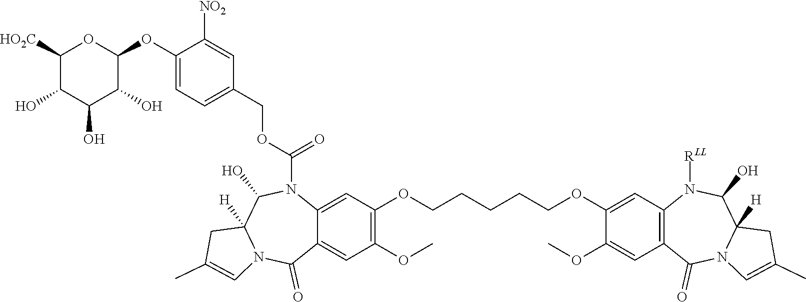 | A5 |
| 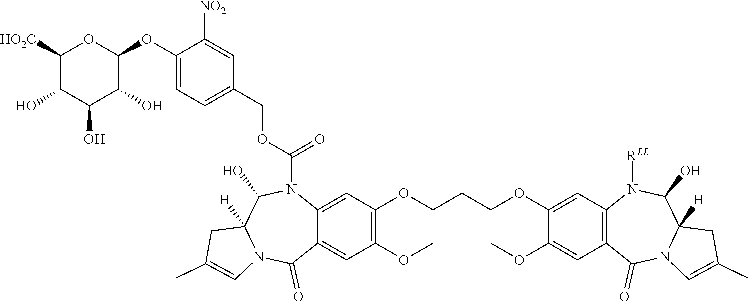 | A6 |
| 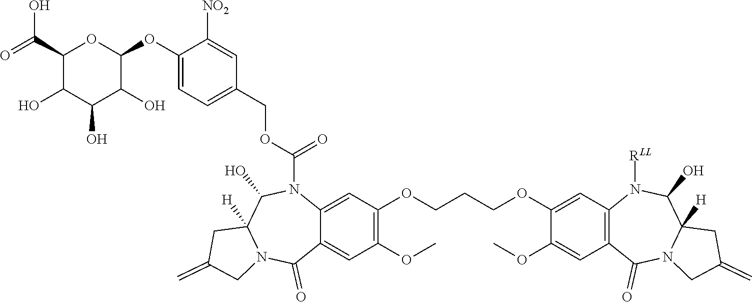 | A7 |

where R^{LL}, X and G^{LL} are as described above.

In some embodiment DL is selected from A1 to A6.

In some embodiments of the disclosure, DL is selected from:

| | |
|---|---|
| 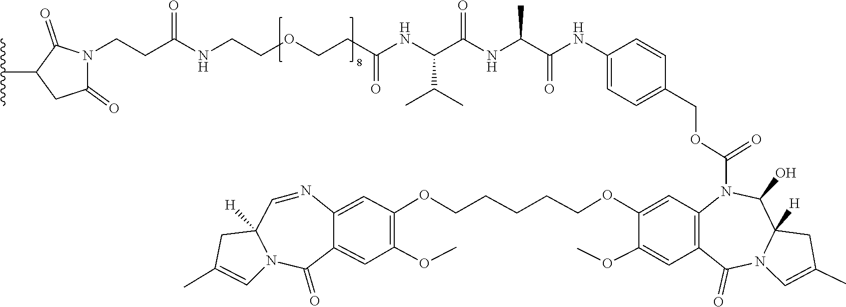 | B1 |
| 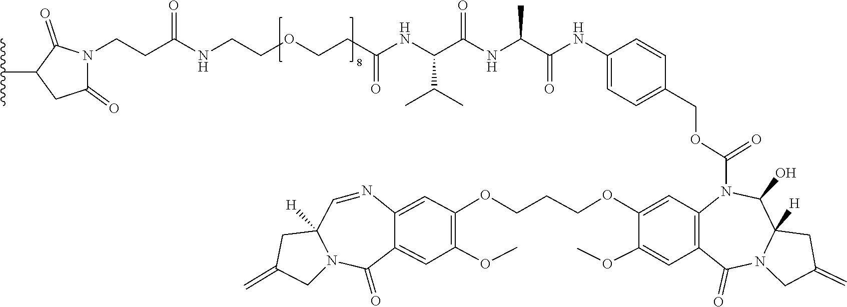 | B2 |
| 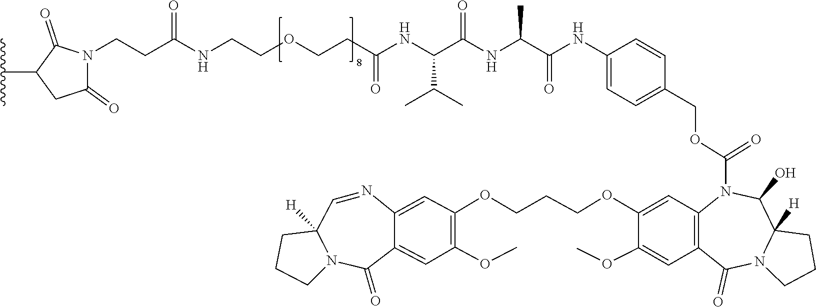 | B3 |
| 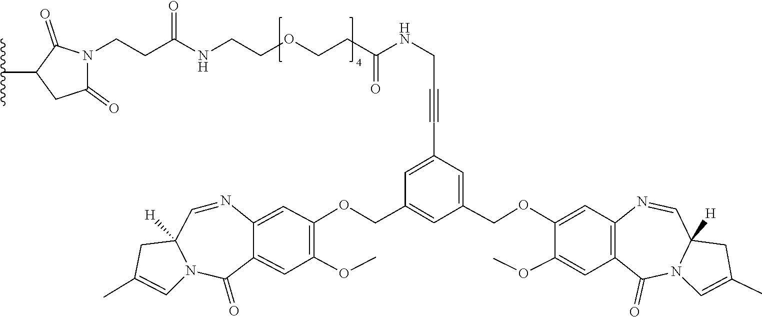 | B4 |
| 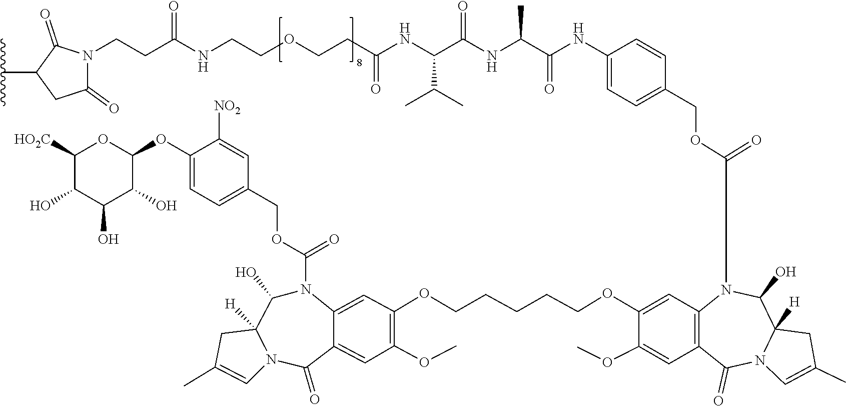 | B5 |
| 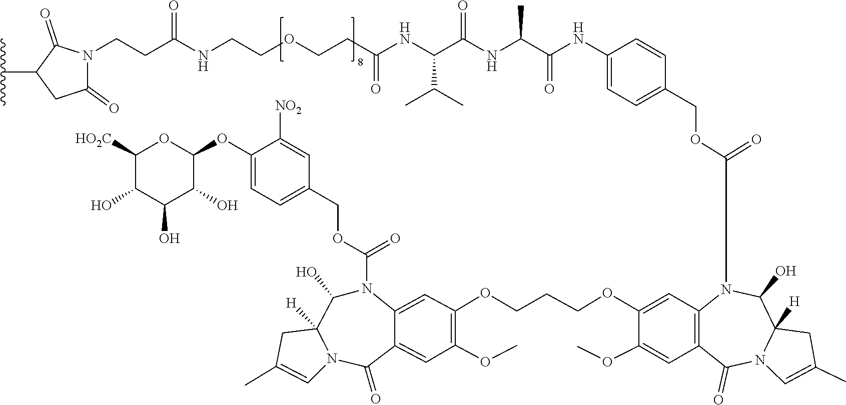 | B6 |
| 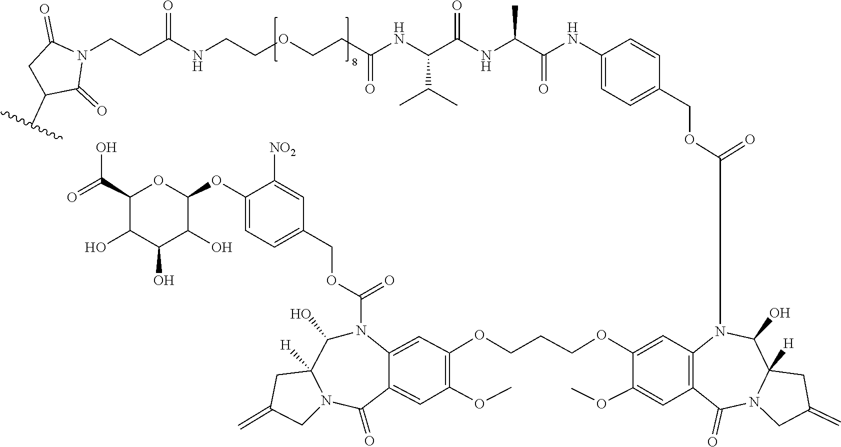 | B7 |
| 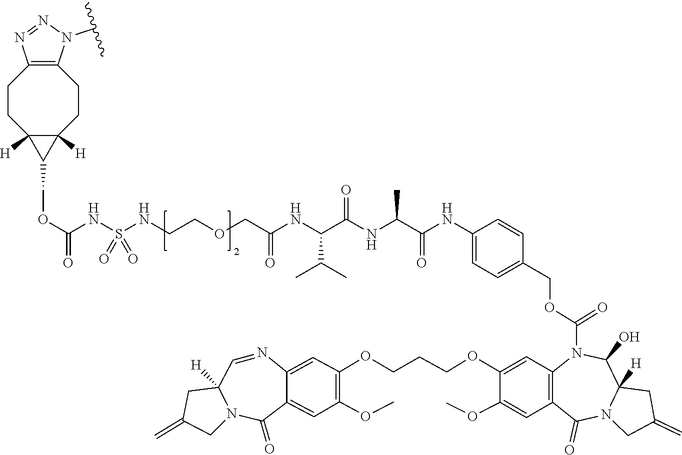 | B8 |
| 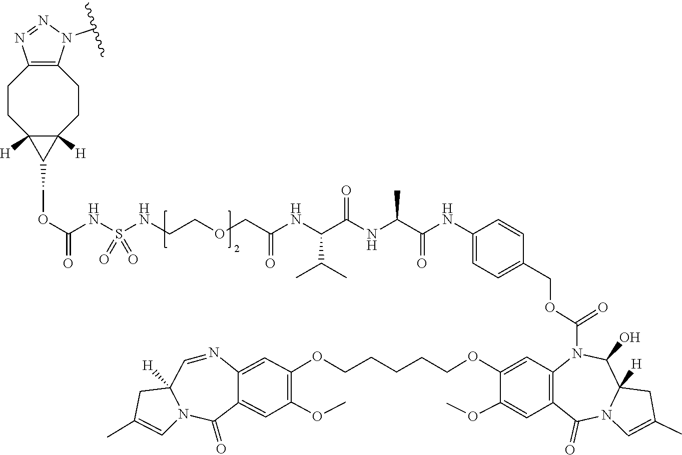 | B9 |

### Drug Loading

The drug loading is the average number of PBD drugs per antibody, e.g. antibody.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For the present antibody-drug conjugates, p is limited by the number of attachment sites on the antibody, i.e. the number of azide groups. For example, the antibody may have only one or two azide groups to which the drug linker may be attached.

Typically, fewer than the theoretical maximum of drug moieties are conjugated to an antibody during a conjugation reaction. The loading (drug/antibody ratio) of an ADC may be controlled in several different manners, including: (i) limiting the molar excess of drug-linker intermediate (D-L) or linker reagent relative to antibody, and (ii) limiting the conjugation reaction time or temperature.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per antibody is in the range 1 to 8. In some embodiments the range is selected from 1 to 4, 1 to 4, 2 to 4, and 1 to 3.

In some embodiments, there are one or two dimer pyrrolobenzodiazepine groups per antibody.

### Preparation of Drug Conjugates

The antibody drug conjugates of the present disclosure may be prepared by conjugating the appropriate drug linker of formula DLA or DLB to Ab:

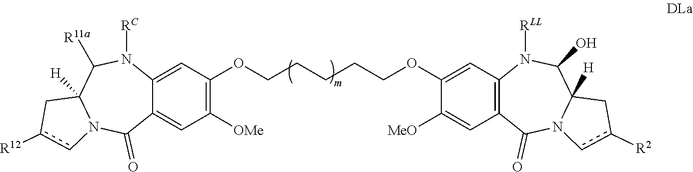

where R^{L} is a linker suitable for connection to Ab, and is of formula IIb:

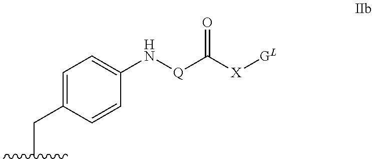

where G^{L} is a linker group suitable for connection to Ab;

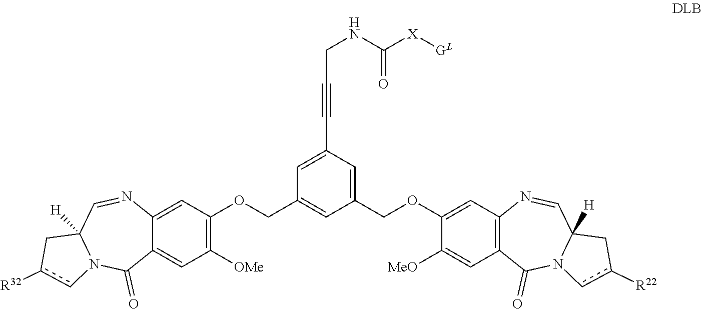

where X and G^{L} are as defined above.

Examples of suitable ADC conjugation methods are set out below in ‘Examples’ section under the sub-heading ‘Preparation of ADCs’.

The drug linker may be synthesised as described in, for example, WO2014/057074, WO2017/137553, WO2018/069490 and WO2018/192944.

In particular, the following table provides references for some of the drug-linkers of particular interest.

| | | |
|---|---|---|
| C1 | Com- pound B in WO 2014/ 057074 | 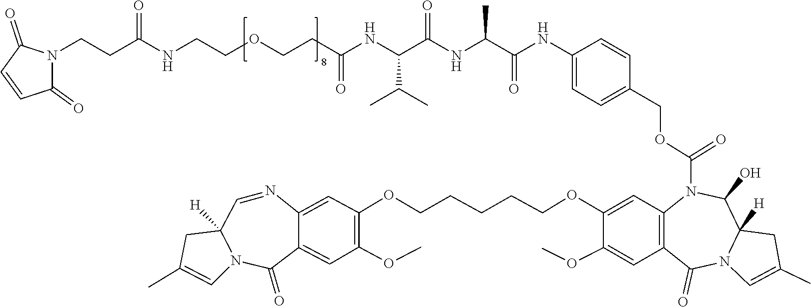 |
| C2 | Com- pounds 23 in WO 2017/ 137553 | 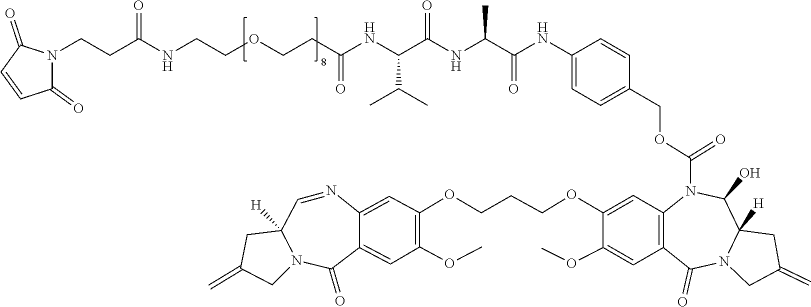 |
| C3 | Com- pound 1 in WO 2018/ 192944 | 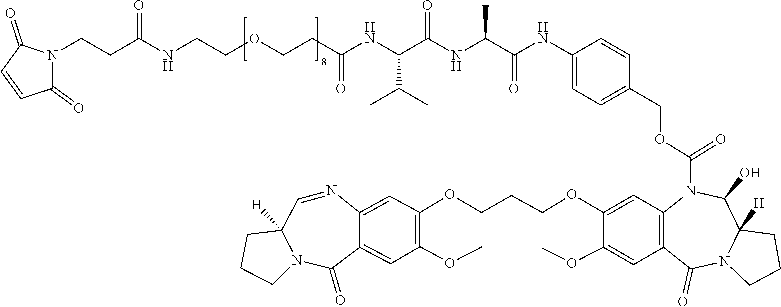 |
| C4 | Com- pound 27 in WO2014/ 140862 | 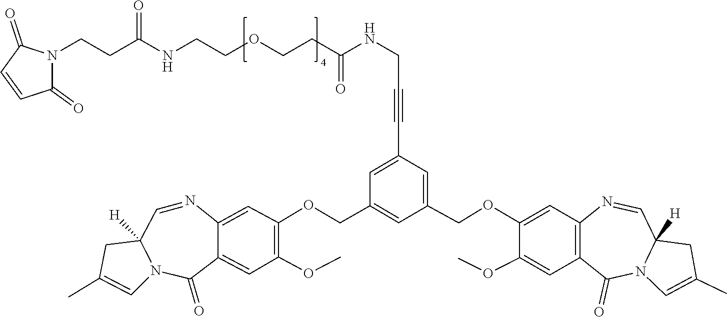 |
| C5 | Com- pound 22 In WO2018/ 069490 | 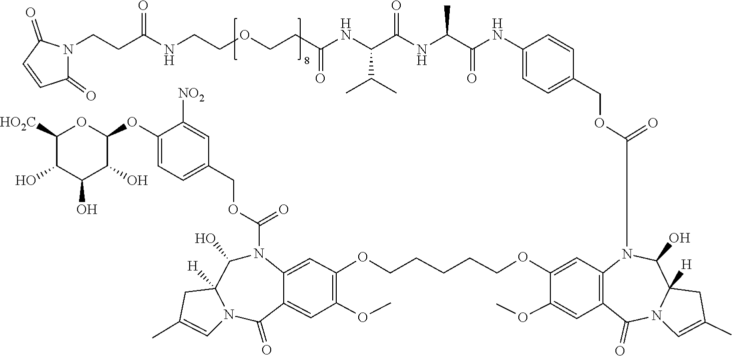 |
| C7 | Com- pound 10 in WO2018/ 069490 | 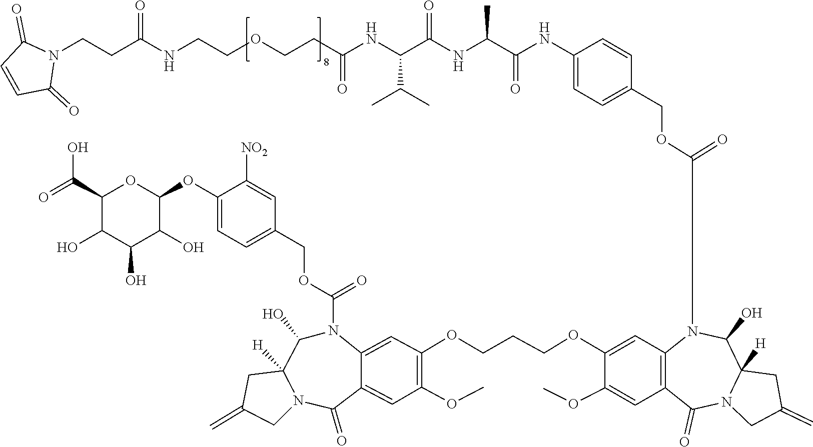 |
| C8 | See below | 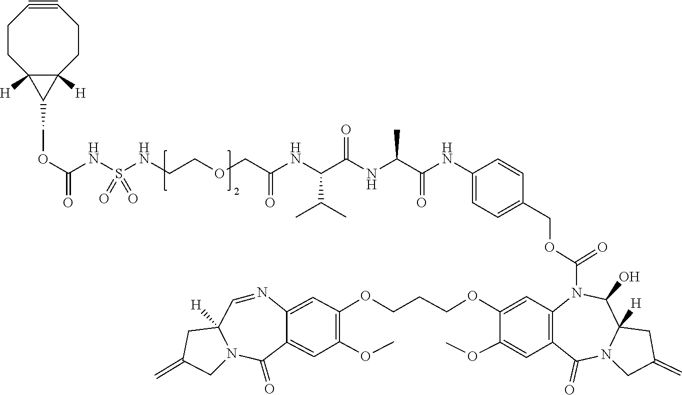 |
| C9 | Com- pound 4 in WO2018/ 146188 | 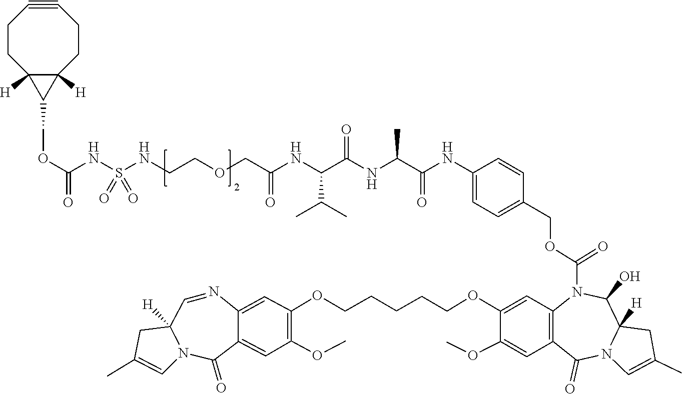 |

### Synthesis of C8

### Analytical LC/MS Conditions

Positive mode electrospray mass spectrometry was performed using a Waters Aquity H-class SQD2. Mobile phases used were solvent A (water with 0.1% formic acid) and solvent B (acetonitrile with 0.1% formic acid).

Method 1: Gradient for routine 3-minute run: Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Detection was at 254 nm. Column: Waters Acquity UPLC™ BEH Shield RP18 1.7 μm 2.1×50 mm at 50° C. fitted with Waters Acquity UPLC™ BEH Shield RP18 VanGuard Pre-column, 130 A, 1.7 μm, 2.1 mm×5 mm.

Method 2: Gradient for 15-minute run: Initial composition 5% B held over 1 minute, then increased from 5% B to 100% B over a 9 minute period. The composition was held for 2 minutes at 100% B, then returned to 5% B in 10 seconds and held there for 2 minutes 50 seconds. The total duration of the gradient run was 15.0 minutes. Flow rate was 0.8 mL/minute (for 3-minute run) and 0.6 mL/minute (for 15-minute run). Detection was at 254 nm. Column: ACE Excel 2 C18-AR, 2μ, 3.0×100 mm fitted with Waters Acquity UPLC™ BEH Shield RP18 VanGuard Pre-column, 130 A, 1.7 μm, 2.1 mm×5 mm.

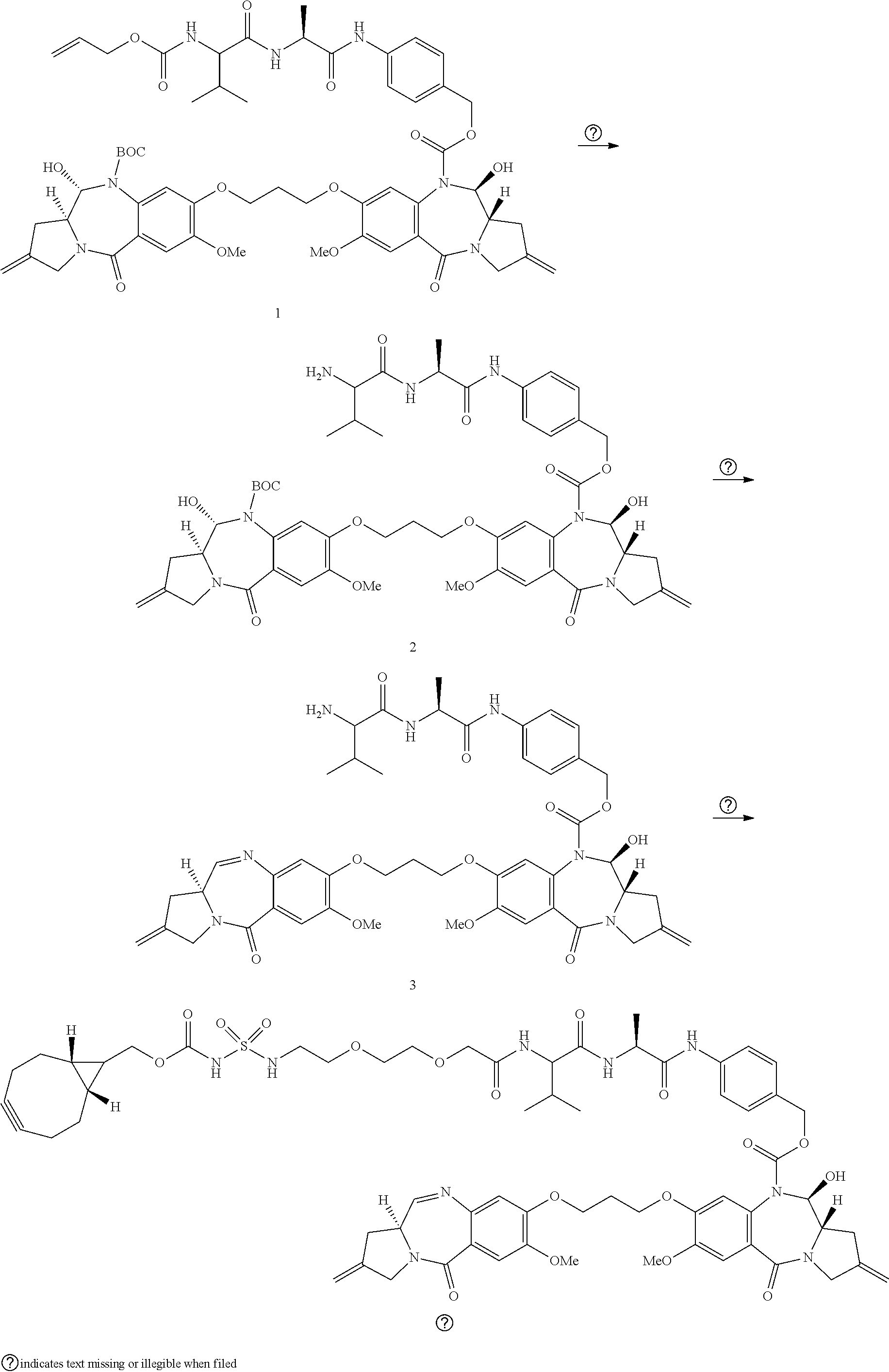

### (i) Alloc Deprotection

Tetrakis(triphenylphosphine)palladium(0) (8.4 mg, 2 mol %) was added to a solution of (1) (400 mg, 0.36 mmol, 1.0 eq)(Compound 21 in WO2017/137553) and pyrrolidine (38 μL, 0.46 mmol, 1.25 eq) in chloroform (10 mL). The reaction was stirred for 20 minutes at room temperature, LCMS shows complete reaction. The reaction mixture was diluted with chloroform (5 mL), washed with saturated aqueous ammonium chloride (10 mL) and passed through a Biotage phase separator tube to remove traces of water. Deloxan™ (1 g) was added to the organic phase and stirred at room temperature for 60 mins. The Deloxan was removed by filtration, washed with chloroform (5 mL) and the organic fractions evaporated under reduced pressure to leave 2 as a white solid (335 mg, 94%). LC/MS, method 1, 1.30 min (ES+) m/z 1013.1 ([M+H]⁺).

### (ii) Boc Deprotection

A mixture of TFA (4.5 mL) and water (0.5 mL) was cooled to 0° C. and added to 2 (320 mg, 0.31 mmol). The resulting solution was stirred at 0° C. for 2 hr. Water (5 mL) and chloroform (10 mL) were added and the mixture basified (pH 8) by the addition of solid sodium hydrogen carbonate. The organic phase was removed by passing through a Biotage SPE cartridge, and evaporated to dryness under reduced pressure to leave 3 as an off-white solid (216 mg, 77%). LC/MS, method 1, 1.23 min (ES+) m/z 894.9 ([M+H]⁺).

(iii) BCN Hydraspace™ Coupling

EDCI.HCl (86 mg, 0.45 mmol, 1.1 eq) was added to a solution of 3 (200 mg, 0.22 mmol, 1.0 eq) and BCN spacer (108 mg, 0.26 mmol, 1.15 eq)(compound 3 in WO 2018/146188) in chloroform (10 mL) and the resulting reaction stirred at room temperature for 60 min. LCMS showed no starting material to be present. The organic phase washed with water (10 mL). The resulting mixture was passed through a biotage phase separator to remove the water and evaporated to dryness to leave a yellow solid which was purified by prep HPLC (gradient 30-90% acetonitrile/water over 9 min. The water containing 0.01% formic acid, but no acid in the acetonitrile). The crude material was dissolved in acetonitrile (1.3 mL) and water (0.7 mL) and injected in 100 uL batches. The product was collected in tubes containing 5% aqueous ammonium bicarbonate solution (2 mL). The fractions containing product were combined, the acetonitrile removed under reduced pressure and the resulting aqueous phase extracted with DCM (3×50 mL). The organic fractions were dried by passing through a Biotage phase separator and evaporated to dryness to leave the product C8 as a pale yellow solid (75 mg, 26%). LC/MS, method 2, 6.78 min (ES+) m/z 1295.3 ([M+H]⁺).

### Therapeutic Uses of Anti-CD45 PBD ADCs

The primary therapeutic applications of the antibodies and antibody drug conjugates of the disclosure include in the treatment of cancer, particularly haematological cancers, and in conditioning subjects for bone marrow or haematopoietic stem cell transplant and gene therapy.

As used herein, the term “conditioning” may be understood to mean the process of preparing a subject for transplantation with a preparation containing haematopoietic stem cells, or for gene therapy, by selectively depleting (i.e., by cell killing) the subject's endogenous, autologous haematopoietic stem cells, haematopoietic progenitor cells, and/or leukocytes, to provide a niche for engraftment of the transplanted cells. The anti-CD45 PBD ADCs disclosed herein exert targeted cell killing activity of cells expressing CD45 CD45-positive cells). Typically such cells are those of the haematopoietic system. Thus, the present disclosure provides the anti-CD45 PBD ADCs disclosed herein for use in therapy.

### Haematological Cancer Treatment

The present disclosure provides a method of treating haematological cancer, the method comprising administering an anti-CD45 PBD ADC as described herein, or a composition comprising such an ADC, to a subject in need thereof. Also provided is an anti-CD45 PBD ADC as described herein, or a composition comprising such an ADC, for use in a method of treating haematological cancer, the method comprising administering the ADC or composition to a subject in need thereof. The anti-CD45 PBD ADC may be a single type of ADC as described herein, or may be a combination of a first anti-CD45 PBD ADC as described herein, and a second, different, anti-CD45 PBD ADC as described herein. Similarly the method may comprise administering a first anti-CD45 PBD ADC as described herein to a subject in need thereof, wherein the subject has been, is being, or will be administered a second, different, anti-CD45 PBD ADC as described herein.

In some cases the first anti-CD45 PBD ADC and the second anti-CD45 PBD ADC are administered to the subject within the same pharmaceutical composition. In some other preferred cases, the first anti-CD45 PBD ADC and the second anti-CD45 PBD ADC are administered to the subject as two separate pharmaceutical compositions. In some cases the first anti-CD45 PBD ADC and the second anti-CD45 PBD ADC are administered to the subject concurrently. In some cases the first anti-CD45 PBD ADC is administered to the subject before the second anti-CD45 PBD ADC is administered to the subject. In some cases the first anti-CD45 PBD ADC is administered to the subject immediately before the second anti-CD45 PBD ADC is administered to the subject. In some cases the first anti-CD45 PBD ADC is administered to the subject at least 1, 2, 3, 4, 5, 6, 7, 8, 9, 10, 11, 12, 24, 36, 48, or 72 hours before, preferably at least 2 hours before, most preferably at least 12 hours before, the second anti-CD45 PBD ADC is administered to the subject. In some cases the first anti-CD45 PBD ADC is administered to the subject at least 1, 2, 3, 4, 5, 7, 10, 14, 21, 28, 35, 42, or 49 days before, preferably at least 1 day, most preferably at least 7 days, before the second anti-CD45 PBD ADC is administered to the subject.

In some cases the haematological cancer comprises CD45-expressing cells. In some cases the haematological cancer may be CD45-positive. In some cases, the cancer may be selected from the group consisting of acute myeloid leukaemia, acute lymphoblastic leukaemia, chronic lymphocytic leukaemia, chronic myeloid leukaemia, myelodysplasia, multiple myeloma, non-Hodgkin's lymphoma and Hodgkin's disease. The subject may be a mammal, preferably the subject is a human. Typically the subject is a human (e.g., a patient) in need of treatment for haematological cancer, i.e., a subject having, or suspected of having, haematological cancer. In some cases, the subject has been diagnosed as having haematological cancer, and is therefore in need of treatment as described herein.

In some cases the methods may further comprise administering one or more other therapeutic agents. For example, the methods may further comprise administering one or more anti-cancer agents. In some cases the methods further comprise administering to the subject radiation and/or chemotherapy. In some cases, the methods further comprise administering to the subject one or more of enasidenib, gilteritinib, ivosidenib, midostaurin, fludarabine, cyclophosphamide, rituximab, bendamustine, chlorambucil, ibrutinib, idelalisib, obinutuzumab, ofatumumab, prednisolone, brentuximab vedotin, lenalidomide, pomalidomide, carfilzomib, daratumumab, thalidomide, panobinostat, bortezomib, all-trans retinoic acid, arsenic trioxide, idarubicin, daunorubicin, cytarabine, azacitidine, mitoxantrone, cytarabine, etoposide, gemtuzumab, 5-azacytidine, hydroxyurea, midostaurin, vincristine, steroids, doxorubicin, asparaginase, ifosfamide, methotrexate, nelarabine, melphalan, bendamustine, carmustine (bis-chloroethylnitrosourea, BCNU), cis platin, carboplatin, busulphan, treosulphan, thiotepa, or total body irradiation. In some preferred cases, the methods further comprise administering to the subject one or more cancer treatment(s) selected from: daunorubicin, idarubicin, mitoxantrone, cytarabine, etoposide, fludarabine, gemtuzumab, 5-azacytidine, hydroxyurea, midostaurin, vincristine, steroids, doxorubicin, asparaginase, cyclophosphamide, ifosfamide, methotrexate, nelarabine, daratumumab, melphalan, thalidomide, lenolidamide, bortezimib, pomalidomide, carfolizimib, bendamustine, carmustine (bis-chloroethylnitrosourea, BCNU), cis platin, carboplatin, rituximab, ofatumumab, obinutuzumab, ibrutinib, idelasalib, and brentuximab. In some cases, the methods further comprise administering to the subject one or more conditioning agent(s) selected from: busulphan, treosulphan, thiotepa, and total body irradiation. In such cases, the further therapeutic agents may be formulated within the same pharmaceutical composition as the anti-CD45 PBD ADC described herein; or preferably, the further therapeutic agents may be administered in a separate formulation. The further therapeutic agents may be administered concurrently with the anti-CD45 PBD ADC described herein. The anti-CD45 PBD ADC described herein may be administered before, after or concurrently with the one or more further agents.

### Preparing a Subject for Transplantation of Haematopoietic Stem Cells

The present disclosure also provides a method of preparing a subject for transplantation of haematopoietic stem cells, the method comprising administering an anti-CD45 PBD ADC or composition as described herein to a subject in need thereof. In some cases, the present disclosure provides an anti-CD45 PBD ADC or composition as described herein for use in a method of preparing a subject for transplantation of haematopoietic stem cells, the method comprising administering said antibody, antibody drug conjugate or pharmaceutical composition to a subject in need thereof. Preparing a subject for transplantation of haematopoietic stem cells may comprise, or consist essentially of, conditioning the subject for engraftment of haematopoietic stem cells.

The anti-CD45 PBD ADC or composition as described herein typically result in cell killing of CD45-positive cells. It is usually advantageous, before transplantation with a preparation containing haematopoietic stem cells or gene therapy, to reduce as far as possible the number of immunological effector cells in the subject's body, preferably to eliminate them entirely. Thus, the antibody, antibody drug conjugate or pharmaceutical composition of the disclosure provide broad spectrum cell killing of CD45-positive cells, including T-cells, NK cells, lymphocytes and monocytes. This may minimise graft rejection or graft versus host disease following transplantation.

As discussed above, in some cases, preparing a subject for transplantation with haematopoietic stem cells may comprise conditioning the subject for engraftment of haematopoietic stem cells. Thus, it may mean the process of selectively depleting (i.e., by cell killing) the subject's autologous haematopoietic stem cells and/or leukocytes, to provide a niche for engraftment of transplanted haematopoietic stem cells. In some cases the methods described herein may be myeloablative, non-myeloablative or reduced intensity, preferably reduced toxicity myeloablative conditioning. Typically, the subject's autologous haematopoietic stem cells comprise a defect or mutation, which results in a disorder or disease in the subject. Thus, depleting or substantially eliminating the subject's autologous haematopoietic stem cells and replacing them with corrected or healthy haematopoietic stem cells provides a treatment for the disease.

In some cases, the haematopoietic stem cells for transplantation into the subject are allogeneic. The term “allogeneic” may be understood in the context of the disclosure to mean a donor's haematopoietic stem cells, i.e., the haematopoietic stem cells are isolated or derived from a donor, typically a human donor. The haematopoietic stem cells are not the subject's own haematopoietic stem cells, i.e., they are not derived from the subject. In some cases, the allogeneic haematopoietic stem cells for transplantation into the subject are from a healthy donor, i.e., a donor not having the same disease as the subject, or preferably a donor not having any disease. In preferred cases, the allogeneic haematopoietic stem cells for transplantation into the subject are from a healthy, HLA-matched donor. As used herein, the term “HLA-matched” is used to mean, that the human leukocyte antigen (HLA) types of the donor haematopoietic stem cells are a close match with the subject's HLA types. In some cases, the donor and subject have at least 6, preferably at least 8, and most preferably at least 10 matching HLA markers. In some cases the donor may be haploidentical with the subject (i.e., exactly half of the HLA markers match). In some cases, the donor may be a sibling, parent or child of the subject.

In the methods disclosed herein, said transplantation of allogeneic haematopoietic stem cells may be for treating a malignant disease or disorder. In such cases, the transplantation of allogeneic haematopoietic stem cells may be for treating a disease or disorder selected from the group consisting of: acute myeloid leukaemia, acute lymphoblastic leukaemia, chronic myeloid leukaemia, chronic lymphocytic leukaemia, myelodysplasia, myeloproliferative diseases, non-Hodgkin's lymphoma and Hodgkin's disease. Alternatively, in some other cases, said transplantation of allogeneic haematopoietic stem cells may be for treating a non-malignant disease or disorder. In some of these cases, the transplantation of allogeneic haematopoietic stem cells may be for treating a disease or disorder selected from the group consisting of: severe aplastic anaemia, and other bone marrow failure disorders, a primary immunodeficiency, primary haemphagocytic lymphohistiocytosis, a haemoglobinopathy, and a genetic metabolic disease. In some cases, the transplantation of allogeneic haematopoietic stem cells may be for treating (i) a bone marrow failure disorder such as idiopathic severe aplastic anaemia, Fanconi anaemia, dyskeratosis congenital, severe congenital neutropenia, Schwachmann-Diamond Syndrome, or Diamond Blackfan anaemia; (ii) a primary immunodeficiency such as SCID (eg. newborn SCID), chronic granulomatous disease, Wiskott-Aldrich syndrome, CD40 ligand deficiency, XLP, MHC Class II deficiency, or primary haemophagocytic lymphohistiocytosis; (iii) a haemoglobinopathy such as sickle cell disease, β-thalassaemia major; or (iv) a genetic metabolic disease such as Hurler syndrome, X-linked adrenoleukodystrophy, alpha mannosidosis, or osteopetrosis. In some cases, the transplantation of allogeneic haematopoietic stem cells may be for treating a disease or disorder selected from the group consisting of: severe aplastic anaemia and other bone marrow failure disorders, a haemoglobinopathy, a primary immunodeficiency, a haemoglobinopathy, primary haemophagocytic lymphohistiocytosis, a genetic metabolic disease, Fanconi anaemia, dyskeratosis congenita, severe congenital neutropaenia, Schwachmann-Diamond Syndrome, Diamond-Blackfan syndrome, SCID (eg. newborn SCID), chronic granulomatous disease, Wiskott-Aldrich syndrome, CD40 ligand deficiency, XLP, MHC Class II deficiency, primary haemophagocytic lymphohistiocytosis, ssickle cell disease, β-thalassaemia major, Hurler syndrome, alpha mannosidosis, X-linked adrenoleukodystrophy and osteopetrosis. In such cases, the subject may be a human (e.g., a patient) in need of treatment for one or more of said diseases or disorders, i.e., a subject having, or suspected of having, one or more of said diseases or disorders. In some cases, the subject has been diagnosed as having one or more of said diseases or disorders, and is therefore in need of treatment as described herein.

In some cases, the allogeneic haematopoietic stem cells for transplantation into the subject are genetically-modified. In such cases, the transplantation of genetically-modified allogeneic haematopoietic stem cells may be for gene therapy of the subject. Thus, the present disclosure further provides a method of preparing a subject for transplantation of allogeneic genetically-modified haematopoietic stem cells for gene therapy, the method comprising administering an antibody of the invention, an antibody drug conjugate of the invention, or an pharmaceutical composition of the invention. Typically, the subject's autologous haematopoietic stem cells comprise a deficiency, disease or mutation that results in a disease or disorder. The genetically-modified allogeneic haematopoietic stem cells for transplantation may have been isolated or derived from the donor and treated ex vivo, for example by gene therapy, e.g., by transduction with a viral vector carrying a gene for a desired expression product, or through gene/base editing using for example TALENS or CRISPR technology. Following conditioning with the antibodies, antibody drug conjugates or pharmaceutical compositions of the invention, genetically-modified allogeneic haematopoietic stem cells can be transplanted into the subject, which may be useful for gene therapy for treating a genetic haematological disease or disorder, a primary immunodeficiency or a genetic metabolic disorder. For example, following conditioning with the anti-CD45 PBD ADCs as disclosed herein, genetically-modified allogeneic haematopoietic stem cells can be transplanted into the subject for treating Fanconi anaemia, where autologous HSCs are difficult to harvest, or for treating metachromatic leukodystrophy (MLD), where overexpression may be beneficial, or for CAR T cell therapy.

In some cases, the haematopoietic stem cells for transplantation into the subject are autologous. The term “autologous” may be understood in the context of the disclosure to mean the subject's own haematopoietic stem cells, i.e., haematopoietic stem cells isolated or derived from the subject. In some cases, the haematopoietic stem cells for transplantation into the subject may be the subject's own haematopoietic stem cells. In the some of the methods described herein, the transplantation of autologous haematopoietic stem cells may be for treating a malignant disease or disorder. In some of these cases, the transplantation of autologous haematopoietic stem cells may be for treating a disease or disorder selected from the group consisting of: multiple myeloma, non-Hodgkin's lymphoma, and Hodgkin's disease. In some other cases, the transplantation of autologous haematopoietic stem cells may be for treating an autoimmune disease or disorder. In some such cases, the transplantation of autologous haematopoietic stem cells may be for treating a disease or disorder selected from the group consisting of: multiple sclerosis, systemic sclerosis and systemic lupus erythematosus. In some cases, the transplantation of autologous haematopoietic stem cells may be for treating a disease or disorder selected from the group consisting of: multiple myeloma, non-Hodgkin's lymphoma, Hodgkin's disease, an autoimmune disease or disorder, multiple sclerosis, systemic sclerosis, juvenile inflammatory arthritis, and systemic lupus erythematosus.

In some cases, the haematopoietic stem cells for transplantation into the subject are autologous and genetically-modified. In such cases, the transplantation of genetically-modified autologous haematopoietic stem cells may be for gene therapy of the subject. Thus, the present disclosure further provides a method of preparing a subject for transplantation of autologous genetically-modified haematopoietic stem cells for gene therapy, the method comprising administering an anti-CD45 PBD ADC or composition as described herein. Typically, the subject's autologous haematopoietic stem cells comprise a deficiency, disease or mutation that results in a disease or disorder. The genetically-modified autologous haematopoietic stem cells for transplantation may have been isolated or derived from the subject and treated ex vivo, for example by gene therapy, to correct the deficiency, disease or mutation, so that the haematopoietic stem cells no longer result in the disease or disorder. In some cases, the genetically-modified haematopoietic stem cells for transplantation comprise, or consist essentially of, autologous haematopoietic stem cells (i.e., haematopoietic stem cells isolated from the subject to be treated), which have been genetically modified, e.g., by transduction with a viral vector carrying a gene for a desired expression product, by chimeric antigen receptor (CAR) therapy, or through gene/base editing using for example TALENS or CRISPR technology. For example, one or more viral vector(s) comprising a gene encoding the adenosine deaminase (ADA) or β-globin genes can be used in a known manner to insert these genes into haematopoietic stem cells isolated from a subject having an ADA deficiency or a haemoglobinopathy, respectively. Similarly, gene/base editing using CRISPR technology may be used to silence the BCL11A repressor gene to enable expression of γ-globin in haematopoietic stem cells isolated from a subject having sickle cell disease and/or β-thalassaemia. Following conditioning with the anti-CD45 PBD ADC or composition as described herein, transplantation of such gene-corrected (i.e., genetically modified) autologous haematopoietic stem cells can be curative for these disorders.

Thus, in some cases, the haematopoietic stem cells for transplantation into the subject may be genetically-modified autologous haematopoietic stem cells. In the methods disclosed herein, in some cases, the transplantation of genetically-modified autologous haematopoietic stem cells may be for gene therapy. In some cases, the transplantation of genetically-modified autologous haematopoietic stem cells may be for gene therapy for treating a genetic haematological disease or disorder, a primary immunodeficiency or a genetic metabolic disorder. In some cases, the transplantation of genetically-modified autologous haematopoietic stem cells may be for gene therapy for treating (i) a genetic haematological disease or disorder selected from a haemoglobinopathy, a transfusion dependent haemoglobinopathy, sickle cell disease, β-thalassaemia major, and Fanconi anaemia; (ii) a primary immunodeficiency selected from SCID (eg. newborn SCID), chronic granulomatous disease, primary HLH, and Wiskott-Aldrich syndrome; or (iii) a genetic metabolic disorder selected from Hurler's syndrome, X-adrenoleukodystrophy, and metachromatic leukodystrophy. In some cases, the transplantation of genetically-modified autologous haematopoietic stem cells may be for treating a disease or disorder selected from the group consisting of: a genetic haematological disease or disorder, a primary immunodeficiency, a genetic metabolic disorder, sickle cell disease, β-thalassaemia major, Fanconi anaemia, primary HLH, SCID (eg. newborn SCID), chronic granulomatous disease, Wiskott-Aldrich syndrome, Hurler's syndrome, Sanfilippo disease, X-adrenoleukodystrophy, and metachromatic leukodystrophy.

In some cases, the haematopoietic stem cells are comprised within a composition. Thus, in some cases, the methods or medical uses described herein are for preparing a subject for transplantation with a composition comprising or consisting essentially of haematopoietic stem cells. The composition may be a pharmaceutical composition and may comprise a pharmaceutically acceptable carrier, as described herein. In some cases, the subject is, or is intended to be, subsequently administered haematopoietic stem cells, a population of haematopoietic stem cells, or a composition comprising haematopoietic stem cells, wherein the haematopoietic stem cells are allogeneic, autologous or genetically-modified autologous haematopoietic stem cells as described herein. In some cases, the methods or medical uses described herein further comprise administering to the subject haematopoietic stem cells, which may be allogeneic, autologous or genetically-modified autologous haematopoietic stem cells as described herein. In some cases, the methods or medical uses described herein further comprise administering to the subject a population of haematopoietic stem cells, which may be allogeneic, autologous or genetically-modified autologous haematopoietic stem cells as described herein. In some cases, the methods or medical uses described herein further comprise administering to the subject a composition comprising haematopoietic stem cells and optionally a pharmaceutically acceptable carrier, wherein the haematopoietic stem cells are allogeneic, autologous or genetically-modified autologous haematopoietic stem cells as described herein. In some cases, the administered haematopoietic stem cells engraft in a target tissue of the subject. Preferably, said target tissue is bone marrow.

The present disclosure further provides a method of engrafting stem cells in a subject, the method comprising (a) administering an effective amount of an anti-CD45 PBD ADC or composition as described herein, to the subject; and (b) administering a stem cell population to the subject (preferably to a target tissue of the subject), wherein the administered stem cell population engrafts in a target tissue of the subject. In some cases, the present disclosure provides a method of engrafting stem cells in a subject, the method comprising administering an effective amount of an anti-CD45 PBD ADC or composition as described herein, to the subject; wherein the subject is (or is intended to be) subsequently administered a stem cell population (preferably to a target tissue of the subject), wherein the administered stem cell population will engraft in a target tissue of the subject. In some cases, the present disclosure provides an anti-CD45 PBD ADC or composition as described herein for use in a method of engrafting stem cells in a subject, the method comprising (a) administering an effective amount of anti-CD45 PBD ADC or composition as described herein to the subject; and (b) administering a stem cell population to the subject (preferably to a target tissue of the subject), wherein the administered stem cell population engrafts in a target tissue of the subject. Preferably, the stem cells are haematopoietic stem cells, which may be allogeneic, autologous or genetically-modified autologous haematopoietic stem cells as described herein. Preferably, the target tissue is bone marrow.

A method of engrafting stem cells in a subject may describe the process of depleting or substantially eliminating (e.g., by cell killing) a subject's autologous stem cells, which may comprise a mutation or defect that is causing a disease or disorder in the subject, to make space in the bone marrow stem cell niche for replacement of the subject's autologous stem cells with healthy stem cells. Said healthy stem cells may be from a healthy donor or may be produced from stem cells that have been isolated from the subject and treated by gene therapy to correct the defect or mutation that is causing the disease or disorder in the subject, as described above. The stem cells are typically haematopoietic stem cells. The stem cell population may comprise or consist essentially of haematopoietic stem cells. In some cases, the stem cell population comprises exogenous stem cells, e.g., isolated from a healthy donor. In some cases, the stem cell population comprises the subject's autologous stem cells, e.g., that have been genetically modified to correct a disease or genetic defect. The stem cell population typically comprises healthy or corrected stem cells, preferably healthy or corrected haematopoietic stem cells. The healthy stem cells, preferably healthy haematopoietic stem cells are typically from a healthy donor, as described above. The corrected stem cells, preferably corrected haematopoietic stem cells are typically from the subject (i.e., are autologous) and treated ex vivo with gene therapy to correct the defect or mutation that is causing the disease or disorder in the subject, as described above. The healthy or corrected stem cells are typically transplanted into the subject and engraft in the target tissue of the subject. The target tissue may be any tissue to which the stem cell population may be targeted. The target tissue is preferably bone marrow. Thus, the healthy or corrected stem cells are typically haematopoietic stem cells and once transplanted, integrate into the subject's haematopoietic system, in the bone marrow.

In some preferred cases, the stem cell population is administered to the subject after the anti-CD45 PBD ADC or composition as described herein has cleared or dissipated from the subject's target tissue. This prevent or reduces a cytotoxic effect on the administered stem cell population. Accordingly, in some cases, the stem cell population is administered to the subject after the concentration of the anti-CD45 PBD ADC or composition as described herein in the subject's target tissue has been reduced to an undetectable concentration. The period of time necessary to clear the anti-CD45 PBD ADC or composition as described herein from the subject's target tissue may be determined using routine means available to one of skill in the art, for example, by detecting the concentration of anti-CD45 PBD ADC or composition as described herein in the subject's target tissue. In some cases, the stem cell population is administered to the target tissue of the subject after the anti-CD45 PBD ADC or composition as described herein has substantially cleared from the subject's target tissue. In some cases “substantially cleared” means that the level of anti-CD45 PBD ADC or composition as described herein remaining in the target tissue of the subject does not induce significant cell death in the transplanted stem cell population. For example, the stem cell population may be administered to the target tissue of the subject at least 1, 2, 3, 4, 5, 6, 7, 8, 9, 10, 11, 12, 13, 14, 15, 18, 21 or more days, preferably at least 1 day, most preferably at least 2 days, after the administration of the anti-CD45 PBD ADC or composition as described herein. Too long a period between administration of anti-CD45 PBD ADC or composition as described herein and administration of the new stem cell population would undesirably expose the subject to prolonged duration of neutropaenia. In some cases, the stem cell population may be administered to the subject, e.g., to the target tissue of the subject, from 6 to 72 hours, from 1 to 5 days, from 1 to 7 days, or from 1 to 10 days; preferably from about 1 to about 7 days, after the administration of the anti-CD45 PBD ADC or composition as described herein.

In some cases, the methods or medical uses described herein result in conditioning of a subject's target tissues and engraftment of stem cells and achieve at least about 5-100% donor chimerism, preferably at least about 50-100%, most preferably at least about 80-100% donor chimerism (i.e., percentage of the cells derived from the donor) in the subject's target tissue (e.g., bone marrow) four months post-administration of the stem cell population to the subject. Preferably, the donor chimerism is complete, i.e., at least 95% donor chimerism. In some cases, the donor chimerism is stable high-level mixed chimerism, i.e., at least 50% donor chimerism, which is typically sufficient for cure. Most preferably, the donor chimerism is in both myeloid and lymphoid lineages. The level of engraftment needed may depend on the clinical scenario. For haematological malignancies at least about 50-100%, preferably at least about 80-100%, and most preferably complete donor chimerism, is the aim. For non-malignant disorders, whilst complete donor chimerism is still preferable, mixed chimerism (e.g., 30-70%, preferably 50-70% donor chimerism) is often curative. The level of donor chimerism needed to be curative may depend on the disease. For example, for haemoglobinopathies stable, 30% donor chimerism in myeloid lineage may be curative; for primary immunodeficiencies even 10-20% donor chimerism may be sufficient. In some cases, the methods or medical uses disclosed herein that comprise transplantation of genetically-modified autologous haematopoietic stem cells to the subject, for example for gene therapy, may result in a viral copy number of 0.1-10 copies/cell, preferably 0.5-4 copies/cell and most preferably 1-2 copies/cell, in the relevant cell lineage (e.g., myeloid and/or lymphoid) in the blood. In some cases, the methods of the disclosure result in conditioning of a subject's target tissues and engraftment of stem cells and achieve an engraftment rate of at least 50%, preferably at least 60%, most preferably at least 80%, of subjects treated according to the methods described herein. The methods and compositions described herein may provide an enhanced or improved engraftment efficiency, i.e., the efficiency with which an administered stem cell population (e.g., HSCs) engrafts in the conditioned target tissue of the subject (e.g., bone marrow).

The subject may be a mammal, most preferably a human. The subject may have, may have been diagnosed with, or may be suspected of having, a disease or disorder that can be treated by haematopoietic stem cell transplant. In some cases, the subject may have, may have been diagnosed with, or may be suspected of having, a disease or disorder that can be treated by gene therapy, optionally with haematopoietic stem cell transplant. In some cases, the subject has, has been diagnosed with, or is suspected of having, cancer, preferably a haematological cancer. In some cases, the subject has, has been diagnosed with, or is suspected of having, acute myeloid leukaemia, acute lymphoblastic leukaemia, chronic lymphocytic leukaemia, chronic myeloid leukaemia, myelodysplasia, multiple myeloma, non-Hodgkin's lymphoma and Hodgkin's disease. In some cases, the subject has, has been diagnosed with, or is suspected of having, a non-malignant disease, disorder or condition. In some cases, the subject has, has been diagnosed with, or is suspected of having a disorder or disease selected from the group consisting of: severe aplastic anaemia or other bone marrow failure disorder (such as Fanconi anaemia, dyskeratosis congenita, Schwachmann-Diamond Syndrome, severe congenital neutropaenia, Diamond-Blackfan anaemia), a primary immunodeficiency (such as SCID (e.g., newborn SCID), chronic granulomatous disease, Wiskott-Aldrich syndrome, CD40 ligand deficiency, XLP, MHC Class II deficiency, primary haemophagocytic lymphohistiocytosis), a haemoglobinopathy, preferably a transfusion dependent haemoglobinopathy (such as sickle cell disease, β-thalassaemia major), a genetic metabolic disease (such as Hurler's syndrome, X-linked adrenoleukodystrophy, alpha mannosidosis, osteopetrosis, metachromatic leukodystrophy, Sanfilippo disease), or an autoimmune disorder (such as multiple sclerosis, systemic sclerosis, juvenile inflammatory arthritis, systemic lupus erythematosus).

In the methods or medical uses of the disclosure the subject may be chemorefractory (i.e., cannot be treated with chemotherapy, the subject has a cancer that is not responsive to chemotherapy, such as traditional chemotherapies). In some cases, the subject is contraindicated for chemotherapy and/or radiotherapy. In some cases, the subject has an immunodeficiency, optionally a congenital immunodeficiency or an acquired immunodeficiency. In some cases, the subject has pre-existing organ toxicity (i.e. is too ill for conventional conditioning regimes), an infection, an autoimmune disease, a DNA or telomere repair disorder, or is younger than 1 year. In some cases, the subject is a human adult. In some cases, the subject is over 12 years of age, preferably over 15 years of age, or most preferably over 20 years of age. In some cases, the subject is immunocompetent, i.e., not immunodeficient.

The methods described herein of preparing a subject for transplantation of haematopoietic stem cells, and the methods of engrafting stem cells in a subject, may be useful in the treatment of malignant diseases or disorders. In some cases the malignant disease or disorder is caused by CD45-expressing cells. In some cases, the malignant disease or disorder is cancer. In preferred cases, the malignant disease or disorder is a haematological cancer. In some cases, the malignant disease or disorder is selected from the group consisting of acute myeloid leukaemia, acute lymphoblastic leukaemia, chronic lymphocytic leukaemia, chronic myeloid leukaemia, myelodysplasia, multiple myeloma, non-Hodgkin's lymphoma and Hodgkin's disease.

The methods described herein of preparing a subject for transplantation of haematopoietic stem cells, and the methods of engrafting stem cells in a subject, may be useful in the treatment of non-malignant diseases, disorders or conditions. In some cases the non-malignant disease, disorder or condition is caused by CD45-expressing cells. In some cases, the non-malignant disease, disorder or condition may be selected from the group consisting of: severe aplastic anaemia or other bone marrow failure disorder (such as Fanconi anaemia, dyskeratosis congenita, Schwachmann-Diamond Syndrome, severe congenital neutropaenia, Diamond-Blackfan anaemia), a primary immunodeficiency (such as SCID, newborn SCID, chronic granulomatous disease, Wiskott-Aldrich syndrome, CD40 ligand deficiency, XLP, MHC Class II deficiency, primary haemophagocytic lymphohistiocytosis), a transfusion dependent haemoglobinopathy (such as sickle cell disease, β-thalassaemia major), a genetic metabolic disease (such as Hurler's syndrome, X-linked adrenoleukodystrophy, alpha mannosidosis, osteopetrosis, metachromatic leukodystrophy, Sanfilippo disease) or an autoimmune disorder (such as multiple sclerosis, systemic sclerosis, juvenile inflammatory arthritis, systemic lupus erythematosus). In some preferred cases, the methods described herein of preparing a subject for transplantation of haematopoietic stem cells, and the methods of engrafting stem cells in a subject, may be useful in the treatment of a hemoglobinopathy, Fanconi anaemia, chronic granulomatous disease and/or Hurler's syndrome. In some most preferred cases, the methods described herein of preparing a subject for transplantation of haematopoietic stem cells, and the methods of engrafting stem cells in a subject, may be useful in the treatment of sickle cell anemia, β-thalassemia, and/or SCID (eg. newborn SCID).

In the methods of treatment and medical uses described herein the anti-CD45 PBD ADC or composition as described herein are preferably administered by parenteral administration. Preferred routes of administration for the antibodies, conjugates or compositions in the methods and medial uses of the disclosure include intravenous, intramuscular, intradermal, intraperitoneal, subcutaneous, spinal or other parenteral routes of administration, for example by injection or infusion.

The appropriate dosage of anti-CD45 PBD ADC or composition as described herein in the methods of treatment and medical uses described herein will depend for example on the disease to be treated, the subject group and individual subject requirements, but a skilled person would be readily capable of determining a suitable dosage regime. The anti-CD45 PBD ADC or composition as described herein may be administered to the subject in a single dose in one continuous administration, or as multiple doses over a series of separate administrations. The anti-CD45 PBD ADC or composition as described herein may be administered to the subject once in a single day. A suitable initial dose may be about 1 μg/kg to 15 mg/kg. A suitable daily dosage may range from about 1 μg/kg to 100 mg/kg of subject weight. An exemplary dosage may be in the range of about 0.1 mg/kg to about 10 mg/kg of subject weight. In some cases where multiple doses are administered, these may be administered as separate administrations over a period of 1 week, 2 weeks, 3 weeks, 4 weeks, 2 months, 4 months, 8 months, 12 months, 18 months, 2 years, 3 year, 5 years or more; preferably where the anti-CD45 PBD ADC or composition as described herein are administered in multiple doses, they are administered over a period of 2-4 weeks. An exemplary dosing regimen comprises a course of administering an initial loading dose of about 4 mg/kg, followed by additional doses every week, two weeks, or three weeks of an anti-CD45 PBD ADC or composition as described herein. Other dosage regimens may be useful.

### Combination Therapies

The anti-CD45 PBD ADC or composition as described herein may be used in combination in the methods and medical uses of the disclosure, i.e., the methods of preparing a subject for transplantation of haematopoietic stem cells, and the methods of engrafting stem cells in a subject, which are described herein. The above disclosure of the further features of the methods or medical uses of the disclosure applies equally to the methods or medical uses of the disclosure that use a combination of anti-CD45 PBD ADCs, as described below.

The present disclosure also provides a method of preparing a subject for transplantation of haematopoietic stem cells, the method comprising administering a first anti-CD45 PBD ADC as described herein, and a second, different anti-CD45 PBD ADC as described herein, to a subject in need thereof. The disclosure also provides a method of preparing a subject for transplantation of haematopoietic stem cells, the method comprising administering a first anti-CD45 PBD ADC as described herein to a subject in need thereof, wherein the subject has been, is being, or will be administered a second, different anti-CD45 PBD ADC as described herein.

The present disclosure further provides a first anti-CD45 PBD ADC as described herein and a second, different anti-CD45 PBD ADC as described herein, for use in a method of preparing a subject for transplantation of haematopoietic stem cells, wherein the method comprises administering the first anti-CD45 PBD ADC and the second anti-CD45 PBD ADC to a subject in need thereof. The present disclosure also provides a first anti-CD45 PBD ADC as described herein, for use in a method of preparing a subject for transplantation of haematopoietic stem cells, wherein the method comprises administering the first anti-CD45 PBD ADC, and a second, different anti-CD45 PBD ADC as described herein, to a subject in need thereof. In some cases, the first anti-CD45 PBD ADC may be an ADC comprising an antibody of the YTH24.5 group, as described herein, and the second ADC may be an ADC comprising an antibody of the YTH54.12 group, as described herein. In some cases, the first anti-CD45 PBD ADC may be an ADC comprising an antibody of the YTH54.12 antibody group, as described herein, and the second anti-CD45 PBD ADC may be an ADC comprising an antibody of the YTH24.5 antibody group, as described herein.

In some cases the first anti-CD45 PBD ADC and the second anti-CD45 PBD ADC are administered to the subject within the same pharmaceutical composition. In some other preferred cases, the first anti-CD45 PBD ADC and the second anti-CD45 PBD ADC are administered to the subject as two separate pharmaceutical compositions. In some cases the first anti-CD45 PBD ADC and the second anti-CD45 PBD ADC are administered to the subject concurrently. In some cases the first anti-CD45 PBD ADC is administered to the subject before the anti-CD45 PBD ADC antibody is administered to the subject. In some cases the first anti-CD45 PBD ADC is administered to the subject immediately before the second anti-CD45 PBD ADC is administered to the subject. In some cases the first anti-CD45 PBD ADC is administered to the subject at least 1, 2, 3, 4, 5, 6, 7, 8, 9, 10, 11, 12, 24, 36, 48, or 72 hours before, preferably at least 1 hour before, the second anti-CD45 PBD ADC is administered to the subject. In some cases the first anti-CD45 PBD ADC is administered to the subject at least 1, 2, 3, 4, 5, 7, 10, 14, 21, 28, 35, 42, or 49 days before, preferably at least 1 day, before the second anti-CD45 PBD ADC is administered to the subject. In some preferred cases the first anti-CD45 PBD ADC and the second anti-CD45 PBD ADC are administered to the subject sequentially on the same day.

The present disclosure also provides a method of engrafting stem cells in a subject, the method comprising (a) administering an effective amount of a first anti-CD45 PBD ADC as described herein, and a second, different anti-CD45 PBD ADC as described herein, to the subject; and (b) administering a stem cell population to the subject, preferably a target tissue of the subject, wherein the administered stem cell population engrafts in the target tissue of the subject. The present disclosure also provides a method of engrafting stem cells in a subject, the method comprising (a) administering an effective amount of a first anti-CD45 PBD ADC as described herein to the subject, wherein the subject has been, is being, or will be administered an effective amount of a second, different anti-CD45 PBD ADC as described herein; and (b) administering a stem cell population to the subject, preferably a target tissue of the subject, wherein the administered stem cell population engrafts in the target tissue of the subject. The present disclosure also provides a method of engrafting stem cells in a subject, the method comprising (a) administering an effective amount of a first anti-CD45 PBD ADC as described herein to the subject, wherein (i) the subject has been, is being, or will be administered an effective amount of a second, different anti-CD45 PBD ADC as described herein; and (ii) the subject will be administered a stem cell population, preferably to a target tissue of the subject, wherein the administered stem cell population engrafts in the target tissue of the subject.

The present disclosure further provides a first anti-CD45 PBD ADC as described herein and a second, different anti-CD45 PBD ADC as described herein, for use in a method of engrafting stem cells in a subject, wherein the method comprises (a) administering an effective amount of the first anti-CD45 PBD ADC and the second anti-CD45 PBD ADC to the subject; and (b) administering a stem cell population to the subject, preferably a target tissue of the subject, wherein the administered stem cell population engrafts in the target tissue of the subject. The present invention also provides a first anti-CD45 PBD ADC of the invention, for use in a method of engrafting stem cells in a subject, wherein the method comprises (a) administering an effective amount of the first anti-CD45 PBD ADC, and an effective amount of a second, different anti-CD45 PBD ADC as described herein, to the subject; and (b) administering a stem cell population to the subject, preferably a target tissue of the subject, wherein the administered stem cell population engrafts in the target tissue of the subject.

In some cases, the first anti-CD45 PBD ADC may be an ADC comprising an antibody of the YTH24.5 antibody group, as described herein, and the second anti-CD45 PBD ADC may be an ADC comprising an antibody of the YTH54.12 antibody group, as described herein. In some cases, the first anti-CD45 PBD ADC may be an ADC comprising an antibody of the YTH54.12 antibody group, as described herein, and the second ADC may be an ADC comprising an antibody of the YTH24.5 antibody group, as described herein. In some cases the first anti-CD45 PBD ADC and the second anti-CD45 PBD ADC are administered to the subject within the same pharmaceutical composition. In some other preferred cases, the first anti-CD45 PBD ADC and the second anti-CD45 PBD ADC are administered to the subject as two separate pharmaceutical compositions. In some cases the first anti-CD45 PBD ADC and the second anti-CD45 PBD ADC are administered to the subject concurrently. In some cases the first anti-CD45 PBD ADC is administered to the subject before the second anti-CD45 PBD ADC is administered to the subject. In some cases the first anti-CD45 PBD ADC is administered to the subject immediately before the second anti-CD45 PBD ADC is administered to the subject. In some cases the first anti-CD45 PBD ADC is administered to the subject at least 1, 2, 3, 4, 5, 6, 7, 8, 9, 10, 11, 12, 24, 36, 48, or 72 hours before, preferably at least 1 hour before, the second anti-CD45 PBD ADC is administered to the subject. In some cases the first anti-CD45 PBD ADC is administered to the subject at least 1, 2, 3, 4, 5, 7, 10, 14, 21, 28, 35, 42, or 49 days before, preferably at least 1 day, before the second anti-CD45 PBD ADC is administered to the subject. In some cases the first anti-CD45 PBD ADC and the second anti-CD45 PBD ADC are administered to the subject sequentially on the same day. In some cases, the stem cell population is administered to the subject at least 1, 2, 3, 4, 5, 6, 7, 8, 9, 10, 11, 12, 13, 14, 15, 18, 21 or more days, preferably at least 1 day, most preferably at least 2 days, after the administration of the first anti-CD45 PBD ADC and/or the second anti-CD45 PBD ADC. In some cases, the stem cell population is administered to the subject from 6 to 72 hours, from 1 to 5 days, from 1 to 7 days, or from 1 to 10 days; preferably from 1 to 7 days after the administration of the first anti-CD45 PBD ADC and/or the second anti-CD45 PBD ADC.

The methods and medical uses of the disclosure, i.e., those methods of treatment using a single anti-CD45 PBD ADC or pharmaceutical composition as disclosed herein, or those methods using a combination of two species of the anti-CD45 PBD ADC or pharmaceutical composition as disclosed herein, may further comprise administering one or more other therapeutic agents. For example, the methods may further comprise administering to the subject one or more agents for myelosuppression, and/or agents for immunosuppression. In some cases, the methods may further comprise administering to the subject one or more of alemtuzumab, fludarabine, and/or cyclophosphamide. In some cases, the methods may further comprise administering to the subject one or more of busulphan, treosulphan, thiotepa, total body irradiation. Alternatively, in some cases the methods do not comprise administering further agents for myelosuppression, and/or agents for immunosuppression. In some cases, the methods may further comprise administration of a therapeutically effective amount of one or more chemotherapeutic agents. For example, in some cases, the methods may further comprise administering to the subject one or more of daunorubicin, idarubicin, mitoxantrone, cytarabine, etoposide, fludarabine, gemtuzumab, 5-azacytidine, hydroxyurea, midostaurin, vincristine, steroids, doxorubicin, asparaginase, cyclophosphamide, ifosfamide, methotrexate, nelarabine, daratumumab, melphalan, thalidomide, lenolidamide, bortezimib, pomalidomide, carfolizimib, bendamustine, carmustine (bis-chloroethylnitrosourea, BCNU), cis platin, carboplatin, rituximab, ofatumumab, obinutuzumab, ibrutinib, idelasalib or brentuximab. In such cases, the further agents may be administered concurrently with the anti-CD45 PBD ADCs or pharmaceutical compositions, as described herein. In some cases, the anti-CD45 PBD ADCs or pharmaceutical compositions as disclosed herein may be administered before, after or concurrently with the one or more further agents. The anti-CD45 PBD ADCs or pharmaceutical compositions as disclosed herein may be administered in combination with or sequentially to, for example, cytotoxic agents, anti-cancer agents, tumour targeting antibodies, target therapy, pathway inhibitors, immunosuppressive agent or myelosuppressive agents. In some cases, the methods may further comprise administering to the subject local radiation and/or chemotherapy. In some cases, the methods do not comprise administering to the subject alemtuzumab (an anti-CD52 antibody) or ATG (anti-thymocyte globulin). In some cases, the methods do not comprise administering to the subject fludarabine. In some cases, the methods do not comprise administering to the subject a low dose cyclophosphamide for immunosuppression. In some cases, the methods do not comprise administering to the subject alemtuzumab, ATG, fludarabine and a low dose cyclophosphamide for immunosuppression. In some cases where the anti-CD45 PBD ADC or pharmaceutical composition as disclosed herein are used in a method of engrafting stem cells in a subject, or for preparing a subject for gene therapy, or for transplantation with a preparation containing haematopoietic stem cells, the one or more anti-CD45 PBD ADCs may be administered in combination with radiotherapy, chemotherapy (such as busulphan, treosulphan, melphalan and others), immunosuppressive agents (such as fludarabine, cyclophosphamide, alemtuzumab, or others), anti-microbial agents (e.g., anti-viral, anti-fungal or anti-bacterial agents), GvHD prophylaxis (such as methotrexate, cyclosporine, tacrolimus and/or others) or GvHD treatment (such as glutocorticoids, ibrutinib, ECP, Ruxolitinib Infliximab, sirolimus or others). In some cases of the methods of treatment or medical uses described herein, such further agents are administered (i) before, (ii) concurrently with, and/or (iii) after, the one or more antibodies or antibody drug conjugates, preferably concurrently with and/or after, most preferably after.

### Further Therapeutic Embodiments

In one aspect the present disclosure provides a method of treating a subject prior to haematopoietic stem cell transplantation, which method comprises administration to the subject of an anti-CD45 ADC.

The present disclosure also provides a composition for administration to subjects which comprises an anti-CD45 ADC. Such a composition may contain a pharmaceutically acceptable vehicle or excipient as appropriate.

Also provided by the present disclosure is the use of an anti-CD45 ADC in the preparation of a medicament for treatment of a subject prior to transplantation of haematopoietic stem cells.

In an aspect, the present disclosure provides a method for ablating selected cell populations and conditioning a subject's tissues for engraftment or transplant (e.g., conditioning a human subject for hematopoietic stem cell transplant). In certain embodiments, the methods and compositions disclosed herein are non-myeloablative.

Generally, in proliferative disease to be treated by allogeneic transplant it is desirable to reduce as far as possible the number of immunological effector cells in the subject's body, preferably to eliminate them entirely. In this regard, it can be an advantage of the therapies disclosed herein that CD45 is used as a target antigen as CD45 is not restricted to T-cells: for instance, it is also expressed on NK cells, lymphocytes and monocytes. Accordingly, the broad spectrum of cell killing of the anti-CD45 ADC disclosed herein minimizes graft rejection.

Advantageously, by virtue of their specifically and potently targeting CD45+ve cells the methods, assays and compositions disclosed herein do not cause the significant ‘non-target’ and/or ‘off-target’ toxicities in tissues such as the thymus that have generally been associated with traditional conditioning methods, such as irradiation. For example, relative to traditional non- or minimally-targeted conditioning regimens, in certain embodiments the compositions and methods disclosed herein do not induce neutropenia, thrombocytopenia and/or anemia, yet result in a stable, mixed chimerism that is of therapeutic relevance. That is, in certain embodiments, the methods and compositions disclosed herein are able to selectively ablate or deplete the endogenous stem cell niche of a target tissue (e.g., bone marrow tissue) without depleting or ablating endogenous neutrophils or myeloid cells. In certain embodiments, the methods and compositions disclosed herein cause an increase in mature endogenous neutrophils. In certain cases, the methods and compositions disclosed herein do not deplete or ablate endogenous platelets. In still other embodiments, the methods and compositions disclosed herein do not induce anemia in the subject. Such compositions and methods may be used, for example, to correct, cure or otherwise ameliorate one or more diseases in an affected subject.

In certain embodiments, disclosed herein are methods of conditioning a subject or a subject's target tissues for engraftment, such methods comprising a selective depletion or ablation of an endogenous stem cell (e.g., hematopoietic stem cell, HSC) or progenitor cell (e.g., hematopoietic progenitor cell, HPC) population in a target tissue of the subject by administering to the subject an effective amount of an anti-CD45 PBD ADC. Typically, a proportion the ADC is internalized by the endogenous stem cell population, thereby depleting or ablating the endogenous stem cell population in the target tissue and conditioning the subject for engraftment of a transplanted cell or cell population.

Also disclosed herein are methods of engrafting stem cells in a subject, such methods comprising: (a) administering to the subject an effective amount of an anti-CD45 PBD ADC, wherein typically the ADC is internalized by an endogenous stem cell (e.g., hematopoietic stem cell) or progenitor cell population, thereby selectively depleting or ablating the endogenous stem cell population in a target tissue of the subject; and (b) administering a stem cell population to the target tissue of the subject, wherein the administered stem cell population engrafts in the target tissue of the subject.

In certain cases, also disclosed herein are methods of treating a stem cell disorder in a subject, such methods comprising: (a) administering to the subject an effective amount of an anti-CD45 PBD ADC, wherein typically the ADC is internalized by an endogenous stem cell (e.g., hematopoietic stem cell) or progenitor cell population in a target tissue of the subject, thereby depleting or ablating the endogenous stem cell or progenitor cell population in the target tissue of the subject; and (b) administering a stem cell population to the target tissue of the subject, wherein the administered stem cell population engrafts in the target tissue of the subject.

The methods and compositions disclosed herein may be used to condition a subject's tissues (e.g., bone marrow) for engraftment or transplant and following such conditioning, a stem cell population is administered to the subject's target tissues. In certain cases, the stem cell population comprises an allogenic stem cell population. In some embodiments, the stem cell population comprises the subject's autologous stem cells (e.g., autologous stem cells that have been genetically modified to correct a disease or genetic defect).

In certain embodiments, the methods and compositions disclosed herein cause an increase in granulocyte colony stimulating factor (GCSF). In certain cases, the methods and compositions disclosed herein cause an increase in macrophage colony stimulating factor (MCSF). In certain embodiments, the methods and compositions disclosed herein cause an increase in endogenous myeloid cells. Without wishing to be bound by any particular theory or mechanism of action, the increase in endogenous myeloid cells that is observed following administration of the anti-CD45 PBD ADCs disclosed herein may occur as a result of an increase in the subject's endogenous GCSF and/or MCSF. Accordingly, in certain embodiments, such an increase in endogenous myeloid cells occurs as a result of an increase in granulocyte colony stimulating factor (GCSF) and/or macrophage colony stimulating factor (MCSF) that may occur secondary to the methods and compositions disclosed herein. In certain cases, the methods and compositions disclosed herein do not deplete or ablate endogenous lymphoid cells.

In certain cases, following conditioning of a subject's target tissues in accordance with the methods and compositions disclosed herein the subject's innate immunity is preserved. In certain cases, following conditioning of a subject's tissues in accordance with the methods and compositions disclosed herein the subject's adaptive immunity is preserved. In certain embodiments, the methods and compositions disclosed herein preserve thymic integrity of the subject. Similarly, in some embodiments, the methods and compositions disclosed herein preserve vascular integrity of the subject.

The methods and compositions disclosed herein may be used to condition bone marrow tissue. In certain cases, the anti-CD45 PBD ADCs disclosed herein are useful for non-myeloablative conditioning, for example, bone marrow conditioning in advance of hematopoietic stem cell transplantation.

The methods and compositions disclosed herein may be used to condition any number of target tissues of a subject, including, for example bone marrow tissue. As used herein, the term “target tissue” generally refers to any tissues of a subject to which the compositions and methods disclosed herein may be selectively targeted. In certain embodiments, such target tissues comprise an endogenous population of HSCs or progenitor cells (e.g., the stem cell niche of the bone marrow tissue). In certain embodiments, the target tissue is or comprises a subject's bone marrow tissue.

In certain cases, the compositions and methods of the present disclosure are useful for non-myeloablative conditioning in a subject, for example, bone marrow conditioning in advance of hematopoietic stem cell or progenitor cell transplantation. By selectively targeting CD45 the pre-conditioning methods disclosed herein minimize the incidence and severity of adverse effects. For example, the incidence and severity of adverse effects commonly associated with traditional conditioning regimens, such as mucositis, which may be minimized or in certain instances eliminated. Similarly, conditioning a subject using the methods and compositions disclosed herein minimizes the incidence of life-threatening thrombocytopenia, neutropenia and red blood cell loss, all of which are commonly associated with traditional conditioning methods, which often require both irradiation and cytotoxic drugs. Accordingly, in certain cases the compositions and methods disclosed herein are characterized as being non-myeloablative.

The quantity and timecourse of treatment depends on the efficacy, which may, for example, be assessed by numbers of circulating leukemic cells, or other suitable indicator. If treatment causes rapid lysis of cells, e.g. due to complement C′ activation, a slow rate of administration is preferred. If the effector mechanism is relatively slow, e.g. there is little or no complement-mediated lysis, a more rapid administration may be preferred.

Administration of the antibody may be in physiological saline or glucose solution or other physiologically acceptable solution.

### Terminology

As used herein the term “stem cells” refers to undifferentiated or partially differentiated cells that can differentiate into various types of cells and proliferate indefinitely to produce more of the same stem cell. Typically, stem cells are the earliest type of cell in a cell lineage and are defined by their ability to form multiple cell types (multi potency) and their ability to self-renew. As used herein, “haematopoietic stem cells” refers to stem cells that can differentiate into the hematopoietic lineage and give rise to all blood cell types such as white blood cells and red blood cells, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). Human hematopoietic stem cells can be identified, for example by cell surface markers such as CD34+, CD90+, CD49f+, CD38- and CD45RA−. Murine hematopoietic stem cells can be identified, for example by cell surface markers such as CD34-, CD133+, CD48-, CD150+, CD244-, cKit+, Seal+, and lack of lineage markers (negative for B220, CD3, CD4, CD8, MacI, GrI, and TerI 19, among others). The compositions and methods described herein may be useful for the depletion or ablation any stem cell, including, but not limited to, peripheral blood stem cells, bone marrow stem cells, umbilical cord stem cells, genetically modified stem cells, etc.

As used herein, the term “hematopoietic progenitor cells” encompasses pluripotent cells which are committed to the hematopoietic cell lineage, generally do not self-renew, and are capable of differentiating into several cell types of the hematopoietic system, such as granulocytes, monocytes, erythrocytes, megakaryocytes, B-cells and T-cells, including, but not limited to, short term hematopoietic stem cells (ST-HSCs), multi-potent progenitor cells (MPPs), common myeloid progenitor cells (CMPs), granulocyte-monocyte progenitor cells (GMPs), megakaryocyte-erythrocyte progenitor cells (MEPs), and committed lymphoid progenitor cells (CLPs). The presence of hematopoietic progenitor cells can be determined functionally as colony forming unit cells (CFU-Cs) in complete methylcellulose assays, or phenotypically through the detection of cell surface markers (e.g., CD45, CD34+, TerI 19-, CD16/32, CD127, cKit, Seal) using assays known to those of skill in the art.

As used herein the terms “ablate” and “ablation” generally refer to the partial or complete removal of a population of cells (e.g., hematopoietic stem cells or progenitor cells) from the target tissues (e.g., bone marrow tissues of a subject). In certain cases, such ablation comprises a complete removal or depletion of such cells from the target tissue. Alternatively, in other cases, such ablation is a partial removal or depletion of such cells (e.g., HSCs or progenitor cells) from the target tissue. For example, in certain cases, the methods and compositions disclosed herein result in at least about 5%, 10%, 12.5%, 15%, 20%, 25%, 30%, 35%, 40%, 45%, 50%, 55%, 60%, 65%, 70%, 75%, 80%, 85%, 90%, 92.5%, 95%, 97.5%, 98% or 99% depletion of the cells (e.g., HSCs or progenitor cells) of the target tissue.

As used herein the terms “internalized” and “internalization” generally mean that the agent and/or toxin are introduced into or otherwise reach the intracellular compartment of one or more cells (e.g., HSCs or progenitor cells) of the target tissue (e.g., bone marrow). For example, an agent and/or toxin may reach the intracellular compartment of a cell via a receptor-mediated process (e.g., an endocytic process) in which the cell will only take in an extracellular agent and/or toxin upon binding to a specific receptor. In certain cases, the agents and/or toxins disclosed herein are internalized by the endogenous stem cell (e.g., HSCs) or progenitor cell population at a rate of at least about 10%, at least about 15%, at least about 20%, at least about 25%, at least about 30%, at least about 40%, at least about 50%, at least about 60%, at least about 70%, at least about 80%, at least about 90%, at least about 95%, or least about 99%.

As used herein the term “effective amount” may be understood to mean and amount of the anti-CD45 PBD ADC or pharmaceutical composition of the disclosure that is sufficient to have the desired effect. Typically it is an amount sufficient to deplete or substantially eliminate the subject's autologous haematopoietic stem cell population. The skilled practitioner is readily capable of determining an effective amount.

In some embodiments, an effective amount of the ADC disclosed herein achieves maximal stem cell depletion (e.g., about 90%, 91%, 92%, 20 93%, 94%, 95%, 96%, 97%, 97.5%, 98%, 99%, 99.5% or more depletion of hematopoietic or progenitor stem cells from the target tissues of the subject). In some embodiments, an effective amount of the ADCs disclosed herein is determined on the basis of a subject's weight. For example, in certain cases, such an effective amount of the ADCs disclosed herein is or comprises one or more doses of ranging between about 200-1 mg/kg.

### Includes Other Forms

Unless otherwise specified, included in the above are the well known ionic, salt, solvate, and protected forms of these substituents. For example, a reference to carboxylic acid (—COOH) also includes the anionic (carbon/late) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

### Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

### Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

The disclosure includes compounds where a solvent adds across the imine bond of the PBD moiety, which is illustrated below where the solvent is water or an alcohol (R^{A}OH, where RA is C₁₋₄ alkyl):

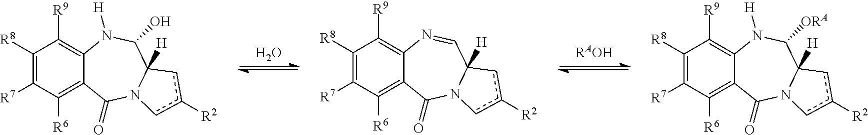

These forms can be called the carbinolamine and carbinolamine ether forms of the PBD (as described in the section relating to R¹⁰ above). The balance of these equilibria depend on the conditions in which the compounds are found, as well as the nature of the moiety itself.

These particular compounds may be isolated in solid form, for example, by lyophilisation.

### Isomers

Certain compounds of the disclosure may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

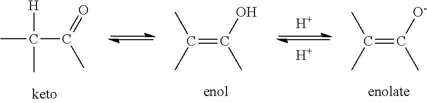

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), 11C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of subjects. Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

### Use

The conjugates of the disclosure may be used to provide a PBD compound at a target location.

The target location is preferably CD45⁺ target cells, including both haematological tumors and haemopietic stem cells.

In one embodiment the antigen is absent or present at a reduced level in a non-proliferative cell population compared to the amount of antigen present in the proliferative cell population, for example a tumour cell population.

At the target location the linker may be cleaved so as to release a PBD compound, such as RelA. Thus, the conjugate may be used to selectively provide a compound RelA to the target location.

The linker may be cleaved by an enzyme present at the target location.

The target location may be in vitro, in vivo or ex vivo.

The antibody-drug conjugate (ADC) compounds of the disclosure include those with utility for anticancer activity. In particular, the compounds include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides a conjugate compound as described herein for use in therapy.

The therapies contemplated herein can comprise, in the case of preparation for haematopoietic stem cell transplant to treat non-proliferative disease (such as non-malignant disease) which is susceptible to such therapy antibody treatment (A) to remove at least a proportion of the stem cells in the marrow, (B) and in most cases to remove (residual) immunocompetent cells from the transplant recipient to reduce the risk of rejection.

In the case of proliferative disease, in particular proliferative haematologic disease, the therapy also has the aim and effect (C) of removing proliferative (eg. leukaemic) cells.

Naturally, effect (B) has no role in otherwise corresponding autologous transplants. In autologous transplants for proliferative disease (such as malignant disease), effects (A) and (C) are relevant. For an autologous transplant of genetically altered progenitor cells, only effect (A) is relevant.

Thus, in various embodiments the aim may be to create a niche in the bone marrow by removing haematopoietic cells, at the same time removing immunologic cells capable of graft rejection. Focus may be on removal of haematopoietic cells in general, including haematopoietic stem cells, and including malignant cells in, for example, leukaemia.

In one aspect the present disclosure provides a conjugate compound as described herein for pre-conditioning subjects prior to HSC transplant. The present therapies are generally applicable to a variety of diseases treatment of which involve transplantation of haematopoietic stem cells. The transplanted cells may be autologous or allogeneic.

A first aspect of the present disclosure provides the use of a conjugate compound in the manufacture of a medicament for treating CD45-expressing proliferative diseases, such as CD45-expressing malignant diseases. In refractory/high risk haematological malignancies, ADCs may enhance the cytoreduction achieved by conventional chemo/radiotherapy conditioning, hence deepening remission prior to transplant and decreasing the risk of relapse. Examples of malignant indications include acute myeloid leukaemia, myelodysplasia, acute lymphoblastic leukaemia, chronic myeloid leukaemia, chronic lymphocytic leukaemia, myeloproliferative disorders, non-Hodgkins lymphoma, Hodgkins disease and multiple myeloma. Treatment of CD45+ve acute myeloid lymphoma (AML) is of particular interest.

The therapies disclosed herein are also applicable in the treatment of non-proliferativce diseases (such as non-malignant diseases), where transplantation of allogenic haematopoietic stem cells is beneficial. Examples of conditions curable by allogeneic stem cell transplant include primary immunodeficiency (eg. SCID, Wiskott-Aldrich syndrome, chronic granulomatous disease, X-linked lymphoproliferative disease and other genetically determined combined immunodeficiency/autoimmune disorders), acquired and congenital (eg Fanconi anaemia, dyskeratosis congenital, severe congenital neutropaenia, Schwachman-Diamond syndrome, Diamond-Blackfan anaemia) bone-marrow failure syndromes, haemoglobinopathies (eg. transfusion-dependent B-thalassaemia, sickle cell disease), haemophagocytic lymphohistiocytosis (HLH), HIV/AIDS, and metabolic disorders (eg. mucopolysccharidoses such as Hurler's syndrome, X-linked adrenoleukodystrophy and osteopetrosis).

The therapies disclosed herein are also applicable in the treatment of non-proliferative autoimmune diseases (such as non-malignant autoimmune diseases), where transplantation of autologous haematopoietic stem cells is beneficial. Examples include autoimmune disorders systemic sclerosis, systemic lupus erythematosus, multiple sclerosis, and juvenile inflammatory arthritis.

The therapies disclosed herein are also applicable in the treatment of genetic disorders where gene therapy of autologous haemopoietic stem cells is beneficial. This treatment may involve transplantation of autologous HSC which have genetically manipulated, e.g. by transduction with a virus vector carrying a gene for a desired expression product or through gene/base editing using for example TALENS or CRISPR technology.

For example, viral vectors encoding the adenosine deaminase (ADA) or β-globin genes can be used in known manner to insert one or both of these genes gene into HSC from subjects with ADA deficiency or haemoglobinopathies respectively. Following conditioning therapy, transplantation of gene-corrected autologous HSCs can be curative for these disorders.

Similarly, gene/base editing using CRISPR technology is being developed to silence the BCL11A repressor gene enabling expression of γ-globin to ameliorate sickle cell disease and β-thalassaemia.

Examples of indications for stem cell gene therapy include a wide variety of primary immunodeficiencies (ADA deficiency, γ-SCID, Wiskott-Aldrich syndrome, X-CGD), metabolic disorders (eg X-linked adrenoleukodystrophy, metachromatic leukodystrophy, Hurler's syndrome) and haemoglobinopathies (β-thalassaemia and sickle cell disease).

For gene therapy to be most effective, conditioning is needed to enable engraftment of gene-corrected/edited autologous HSCs. By virtue of their lack of off target toxicity to non-haemopoeitic tissues, the ADCs disclosed herein substantially broaden the applicability of these technologies, particularly in diseases such as sickle cell anaemia where there has been limited uptake of stem cell transplant up until this point because of the toxicity of conventional condtioning treatments.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. lymphomas and leukemias). Preferably, the ADCS disclosed herein are used to deepen remission in haematological malignancies and to target HSC enabling transplant/gene therapy in non-malignant conditions.

Other disorders of interest include any condition in which CD45 is overexpressed, or wherein CD45 antagonism will provide a clinical benefit. These may include immune disorders, or proliferative diseases such as cancer, particularly metastatic cancer.

It is contemplated that the antibody-drug conjugates (ADC) of the present disclosure may be used to treat various diseases or disorders, e.g. disease or disorders characterized by the (over)expression of CD45. Exemplary conditions or hyperproliferative disorders include benign or malignant tumors; leukemia, haematological, and lymphoid malignancies. Others include neuronal, glial, astrocytal, hypothalamic, glandular, macrophagal, blastocoelic, inflammatory, angiogenic and immunologic, including autoimmune, disorders.

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies.

The compositions and methods disclosed herein may be used to treat or cure a subject having a disease (e.g., a stem cell disorder) that may benefit from hematopoietic stem cell or progenitor cell transplantation (e.g., sickle cell disease), including, for example autologous, allogeneic, gene-modified and gene-therapy methods. As used herein, the phrase “stem cell disorder” broadly refers to any disease, disorder or condition that may be treated or cured by conditioning a subject's target tissues, and/or by ablating an endogenous stem cell population in a target tissue (e.g., ablating an endogenous HSC or progenitor cell population from a subject's bone marrow tissue) and/or by engrafting or transplanting stem cells in a subject's target tissues.

In certain cases, the anti-CD45 PBD ADCs disclosed herein may be used to induce solid organ transplant tolerance (e.g., inducing immunogenic tolerance in connection with kidney transplant). In such embodiments, the ADCs and methods disclosed herein may be used to deplete or ablate a population of cells from a target tissue (e.g., to deplete HSCs from the bone marrow stem cell niche). Following such depletion of cells from the target tissues, a population of stem or progenitor cells from the organ donor (e.g., HSCs from the organ donor) may be administered to the transplant recipient and following the engraftment of such stem or progenitor cells, a temporary of stable mixed chimerism achieved, thereby enabling long-term transplant organ tolerance without the need for further immunosuppressive agents.

### Methods of Treatment

The conjugates of the present disclosure may be used in a method of therapy. Also provided is a method of treatment, comprising administering to a subject in need of treatment a therapeutically-effective amount of a conjugate compound of the disclosure. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound of the disclosure may be administered alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Formulations

While it is possible for the conjugate compound to be used (e.g., administered) alone, it is often preferable to present it as a composition or formulation.

In one embodiment, the composition is a pharmaceutical composition (e.g., formulation, preparation, medicament) comprising a conjugate compound, as described herein, and a pharmaceutically acceptable carrier, diluent, or excipient.

In one embodiment, the composition is a pharmaceutical composition comprising at least one conjugate compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

Another aspect of the present disclosure pertains to methods of making a pharmaceutical composition comprising admixing at least one [¹¹C]-radiolabelled conjugate or conjugate-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the conjugate compound, and compositions comprising the conjugate compound, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

The dosage amounts described above may apply to the conjugate (including the PBD moiety and the linker to the antibody) or to the effective amount of PBD compound provided, for example the amount of compound that is releasable after cleavage of the linker.

For the prevention or treatment of disease, the appropriate dosage of an ADC of the disclosure will depend on the type of disease to be treated, as defined above, the severity and course of the disease, whether the molecule is administered for preventive or therapeutic purposes, previous therapy, the subject's clinical history and response to the antibody, and the discretion of the attending physician. The molecule is suitably administered to the subject at one time or over a series of treatments. Depending on the type and severity of the disease, about 1 μg/kg to 15 mg/kg (e.g. 0.1-20 mg/kg) of molecule is an initial candidate dosage for administration to the subject, whether, for example, by one or more separate administrations, or by continuous infusion. A typical daily dosage might range from about 1 μg/kg to 100 mg/kg or more, depending on the factors mentioned above. An exemplary dosage of ADC to be administered to a subject is in the range of about 0.1 to about 10 mg/kg of subject weight. For repeated administrations over several days or longer, depending on the condition, the treatment is sustained until a desired suppression of disease symptoms occurs. An exemplary dosing regimen comprises a course of administering an initial loading dose of about 4 mg/kg, followed by additional doses every week, two weeks, or three weeks of an ADC. Other dosage regimens may be useful. The progress of this therapy is easily monitored by conventional techniques and assays.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

### The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In preferred embodiments, the subject/patient is human.

In certain cases, the subject is immunocompetent. Alternatively, in certain embodiments, the subject is immunocompromised or immunodeficient. The immunodeficiency may be congenital immunodeficiency. The immunodeficiency may be an acquired immunodeficiency, such as HIV/AIDS.

### STATEMENTS OF INVENTION

In addition to other aspects and embodiments disclosed herein, the following are specifically contemplated.

1. A conjugate of formula (I):

$$Ab - {( DL )}_{p} \qquad ( I )$$

wherein:
Ab is an antibody that binds to CD45;
L is a linker connecting Ab to D, optionally wherein the linker is a non-cleavable linker;
D is a pyrrolobenzodiazepine (PBD), such as a PBD dimer;
and p is from 1 to 8.
   2. The conjugate of claim 1, wherein:
   DL is either:

### (a) DLa:

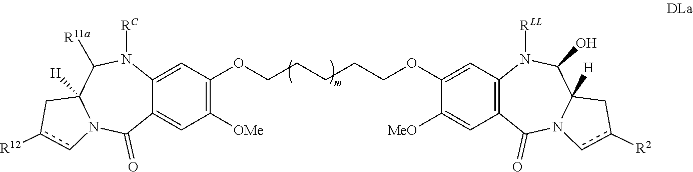

wherein:
R^{LL} is a linker for connection to Ab, which is

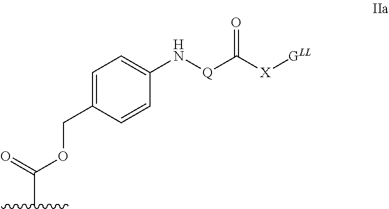

### Wherein

### Q is:

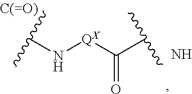

where Q^{X} is such that Q is an amino-acid residue, a dipeptide residue, a tripeptide residue or a tetrapeptide residue;

### X is:

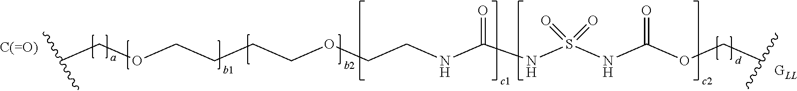

where a=0 to 5, b1=0 to 16, b2=0 to 16, c1=0 or 1, c2=0 or 1, d=0 to 5, wherein at least b1 or b2=0 and at least c1 or c2=0;
G^{LL} is a linker group connected to Ab;
either:
a) R^{11a} and R^{C} together form a double bond between the C and N atoms to which they are attached; or
b) R^{11a} is OH and R^{C} is:

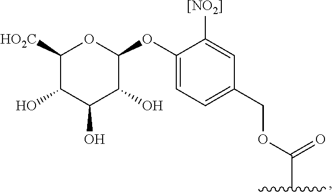

wherein the square brackets indicate the NO₂ group is optional;
m is 0 or 1;
when there is a double bond between C2 and C3, R² is methyl;
when there is a single bond between C2 and C3, R² is either H or

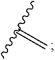

when there is a double bond between C2′ and C3′, R¹² is methyl;
when there is a single bond between C2′ and C3′, R¹² is H or

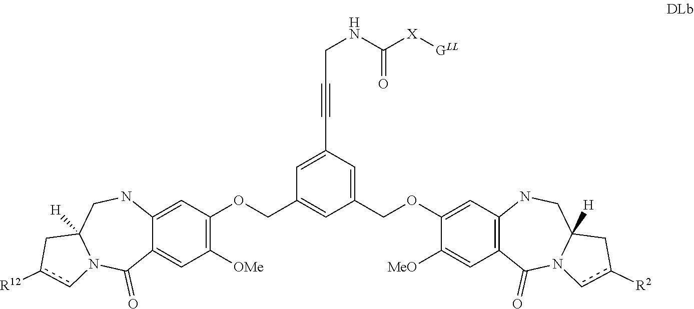

or

### (b) DLb:

where X and G^{LL} are as defined above;
when there is a double bond between C2 and C3, R²² is methyl;
when there is a single bond between C2 and C3, R²² is either H or

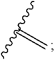

when there is a double bond between C2′ and C3′, R³² is methyl;
when there is a single bond between C2′ and C3′, R³² is H or

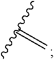

and
and p is from 1 to 8.
3. The conjugate according to statement 2, wherein Q is an amino acid residue.
4. The conjugate according to statement 3, wherein Q is selected from: Phe, Lys, Val, Ala, Cit, Leu, Ile, Arg, and Trp.
5. The conjugate according to statement 2, wherein Q is a dipeptide residue.
6. The conjugate according to statement 5, wherein Q is selected from: ^{C═O}-Phe-Lys-^{NH}; ^{C═O}-Val-Ala-^{NH}, ^{C═O}-Val-Lys-^{NH}, ^{C═O}-Ala-Lys-^{NH}, ^{C═O}-Val-Cit-^{NH}; ^{C═O}-Phe-Cit-^{NH}; ^{C═O}-Leu-Cit-^{NH}; ^{C═O}-Ile-Cit-^{NH}; ^{C═O}-Phe-Arg-^{NH}; ^{C═O}-Trp-Cit-^{NH}, and ^{C═O}-Gly-Val-^{NH}.
7. The conjugate according to statement 6, wherein the dipeptide is selected from: ^{C═O}-Phe-Lys-^{NH}, ^{C═O}-Val-Ala-^{NH}, ^{C═O}-Val-Lys-^{NH}, ^{C═O}-Ala-Lys-^{NH}; and ^{C═O}-Val-Cit-^{NH}.
8. The conjugate according to statement 7, wherein the dipeptide is selected from ^{C═O}-Phe-Lys-^{NH}, ^{C═O}-Val-Cit-^{NH} or ^{C═O}-Val-Ala-^{NH}.
9. The conjugate according to statement 2, wherein Q is a tripeptide residue.
10. The conjugate according to statement 9, wherein Q is selected from: ^{C═O}-Glu-Val-Ala-^{NH}; ^{C═O}Glu-Val-Cit-^{NH}; ^{C═O}-αGlu-Val-Ala-^{NH}; and ^{C═O}-αGlu-Val-Cit-^{NH}.
11. The conjugate according to statement 2, wherein Q is a tetrapeptide residue.
12. The conjugate according to statement 11, wherein the tetrapeptide is selected from:
^{C═O}-Gly-Gly-Phe-Gly-^{NH}; and
^{C═O}-Gly-Phe-Gly-Gly-^{NH}.
   13. The conjugate according to statement 12, wherein the tetrapeptide is:
^{C═O}-Gly-Gly-Phe-Gly-^{NH}
   14. The conjugate according to any one of statements 2 to 13, wherein G^{LL} is selected from:

| | |
|---|---|
| 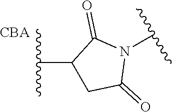 [SMILES: *C1CC(=O)N(*)C1=O] | (G^{LL1-1}) |
| 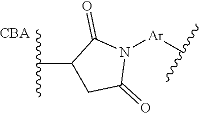 | (G^{LL1-2}) |
| 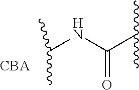 [SMILES: *NC(*)=O] | (G^{LL2}) |
| 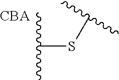 [SMILES: *S*] | (G^{LL3-1}) |
| 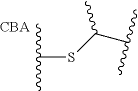 [SMILES: *SC(*)C] | (G^{LL3-2}) |
| 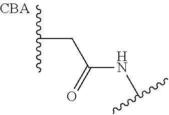 [SMILES: *CC(=O)N*] | (G^{LL-4}) |
| 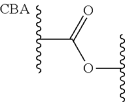 [SMILES: *OC(*)=O] | (G^{LL5}) |
| 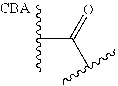 [SMILES: *C(*)=O] | (G^{LL6}) |
| 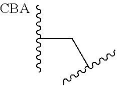 [SMILES: *C*] | (G^{LL7}) |
| 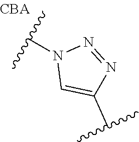 [SMILES: *c1cn(*)nn1] | (G^{LL8-1}) |
| 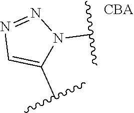 [SMILES: *c1cnnn1*] | (G^{LL8-2}) |
| 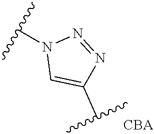 [SMILES: *c1cn(*)nn1] | (G^{LL9-1}) |
| 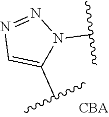 [SMILES: *c1cnnn1*] | (G^{LL9-2}) |
| 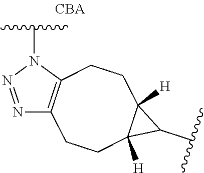 [SMILES: *C1[C@H]2CCc3c(nnn3*)CC[C@@H]12] | (G^{LL10}) |
| 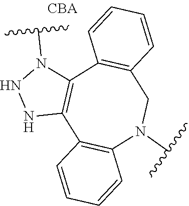 [SMILES: *N1NNC2=C1c1ccccc1CN(*)c1ccccc12] | (G^{LL11}) |
| 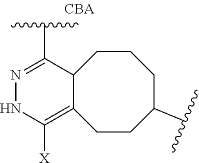 | (G^{LL12}) |
| 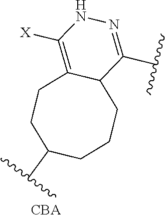 | (G^{LL13}) |
| 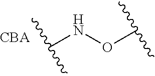 [SMILES: *NO*] | (G^{LL14}) |

where Ar represents a C5-6 arylene group and X represents C₁₋₄ alkyl.
15. The conjugate according to statement 14, wherein G^{LL} is selected from G^{LL1-1} and G^{LL1-2}.
16. The conjugate according to statement 15, wherein G^{LL} is G^{LL1-1}.
17. The conjugate according to any one of statements 2 to 16, wherein a is 0 to 3.
18. The conjugate according to statement 17, wherein a is 0 or 1.
19. The conjugate according to statement 18, wherein a is 0.
20. The conjugate according to statement 19, wherein a is 1.
21. The conjugate according to any one of statements 2 to 20, wherein b1 is 0 to 12.
22. The conjugate according to statement 21, wherein b1 is 0 to 8.
23. The conjugate according to statement 22, wherein b1 0, 2, 3, 4, 5 or 8.
24. The conjugate according to statement 23, wherein b1 is 2.
25. The conjugate according to any one of statements 2 to 20, wherein b2 is 0 to 12.
26. The conjugate according to statement 25, wherein b2 is 0 to 8.
27. The conjugate according to statement 26, wherein b2 is 0, 2, 4 or 8.
28. The conjugate according to statement 27, wherein b2 is 8.
29. The conjugate according to any one of statements 1 to 28, wherein c1 is 1.
30. The conjugate according to any one of statement 1 to 28, wherein c2 is 1.
31. The conjugate according to any one of statements 1 to 30, wherein d is 0 to 3.
32. The conjugate according to statement 32, wherein d is 1 or 2.
33. The conjugate according to statement 32, wherein d is 2.
34. The conjugate according to any one of statements 1 to 30, wherein d is 5.
35. The conjugate according to any one of statements 2 to 16, wherein a is 0, b1 is 0, c1 is 1, c2 is 0 and d is 2, and b2 is from 0 to 8.
36. The conjugate according to statement 35, wherein b2 is 0, 4, 5 or 8.
37. The conjugate according to statement 36, wherein b2 is 8.
38. The conjugate according to any one of statements 2 to 16, wherein a is 1, b2 is 0, c1 is 0, c2 is 1, d is 2, and b1 is from 0 to 8.
39. The conjugate according to statement 38, wherein b1 is 2.
40. The conjugate according to any one of statements 2 to 39, wherein m is 0.
41. The conjugate according to any one of statements 2 to 39, wherein m is 1.
42. The conjugate according to any one of statement 2 to 41, wherein R^{11a} and R^{C} together form a double bond between the C and N atoms to which they are attached.
43. The conjugate according to any one of statement 2 to 41, wherein R^{11a} is OH and R^{C} is:

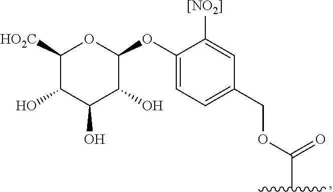

wherein the square brackets indicate the NO₂ group is optional.
44. The conjugate according to statement 43, wherein the NO₂ group is present.
45. The conjugate according to any one of statements 2 to 44, wherein R² and R¹² are the same.
46. The conjugate according to statement 45, wherein there is a double bond between C2 and C3 and between C2′ and C3, and R² and R¹² are both methyl.
47. The conjugate according to statement 45, wherein there is a single bond between C2 and C3 and between C2′ and C3, and R² and R¹² are both H.
48. The conjugate according to statement 45, wherein there is a single bond between C2 and C3 and between C2′ and C3, and R² and R¹² are both

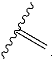

49. The conjugate according to any one of statements 2 to 42, wherein R²² and R³² are the same.
50. The conjugate according to statement 49, wherein there is a double bond between C2 and C3 and between C2′ and C3, and R²² and R³² are both methyl.
51. The conjugate according to statement 49, wherein there is a single bond between C2 and C3 and between C2′ and C3, and R²² and R³² are both H.
52. The conjugate according to statement 49, wherein there is a single bond between C2 and C3 and between C2′ and C3, and R²² and R³² are both

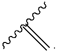

53. The conjugate according to any one of statements 2 to 39, wherein DL is selected from:

| | |
|---|---|
| 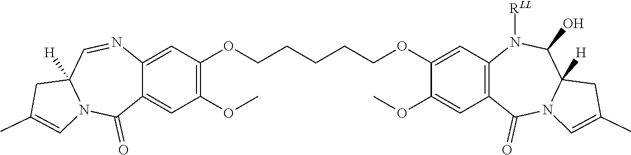 | A1 |
| 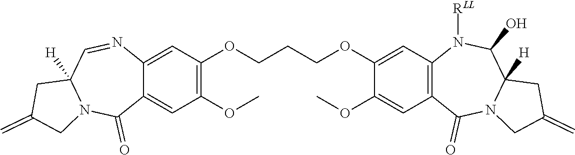 | A2 |
| 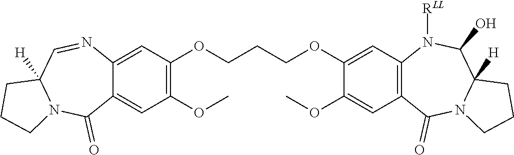 | A3 |
| 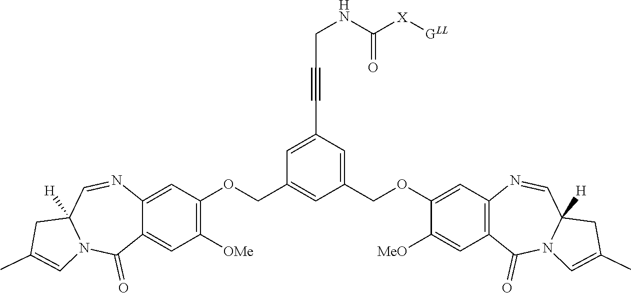 | A4 |
| 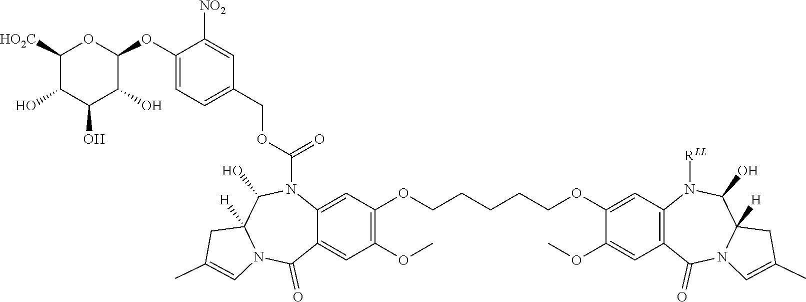 | A5 |
| 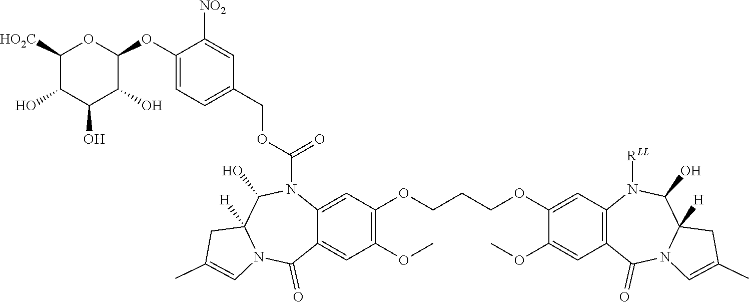 | A6 |
| 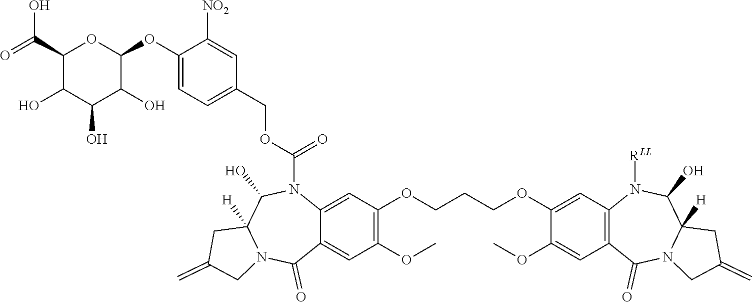 | A7 |

54. The conjugate according to any one of statements 2 to 39, wherein DL is selected from:

| | |
|---|---|
| 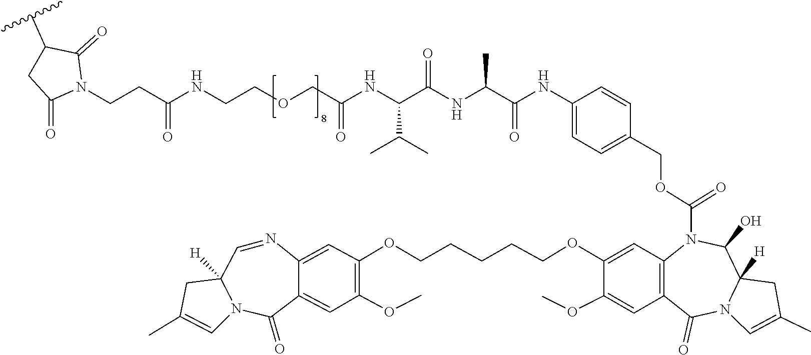 | B1 |
| 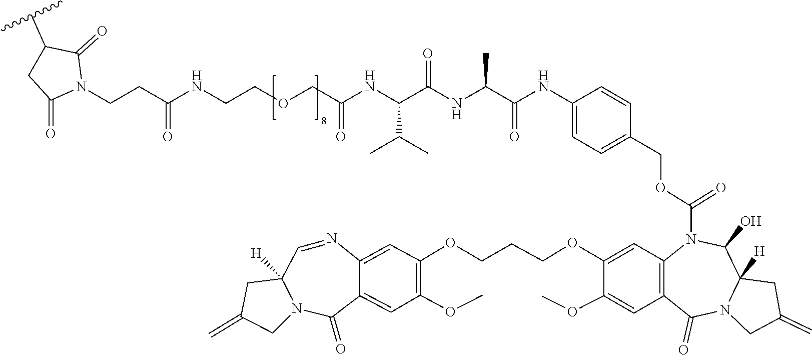 | B2 |
| 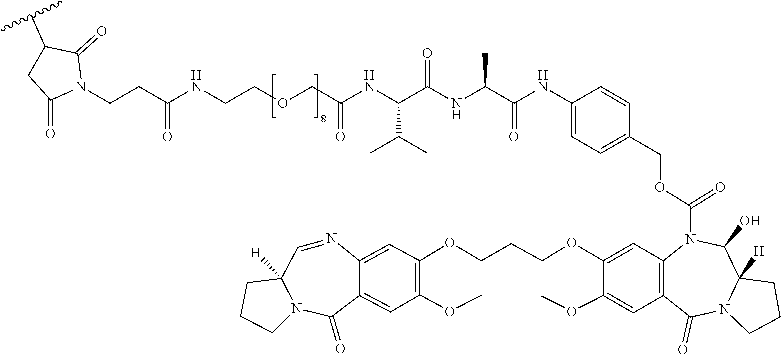 | B3 |
| 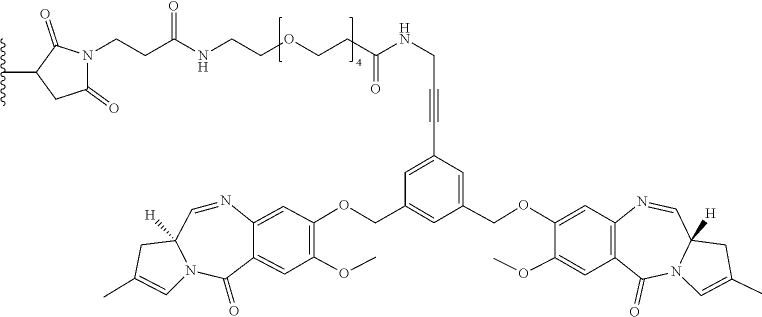 | B4 |
| 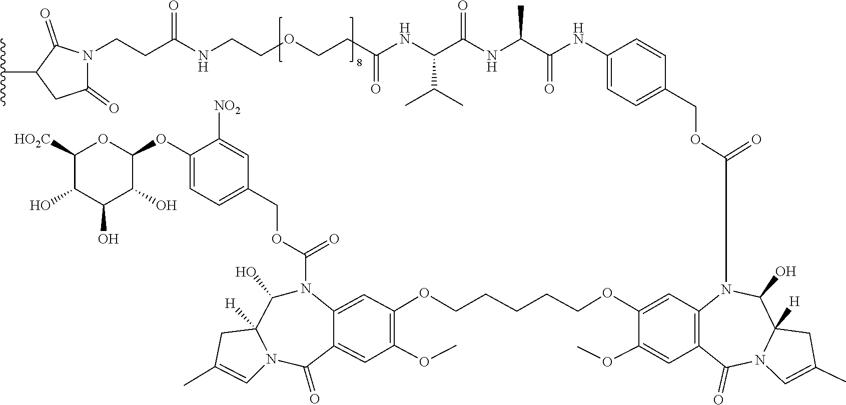 | B5 |
| 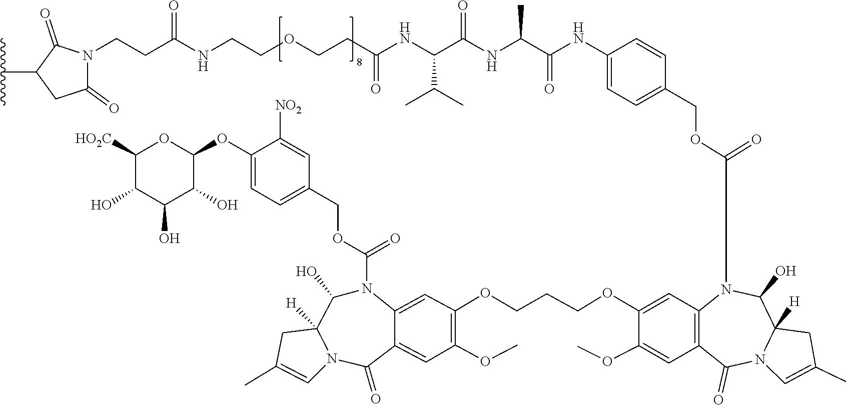 | B6 |
| 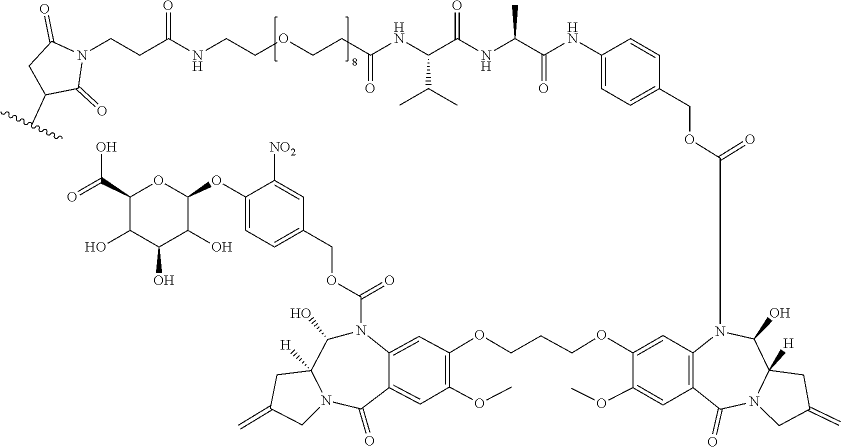 | B7 |
| 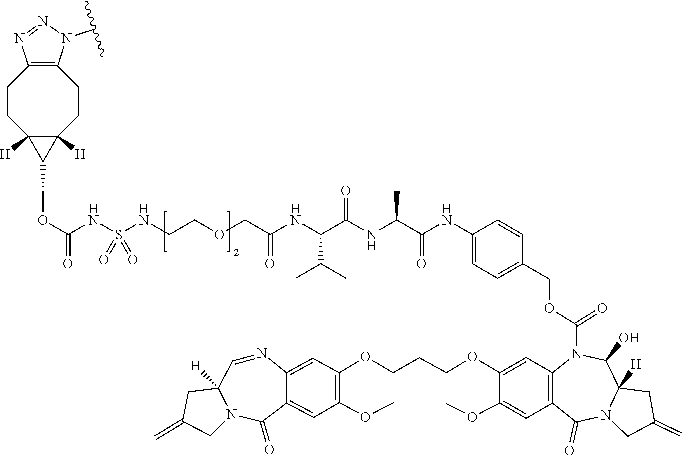 | B8 |
| 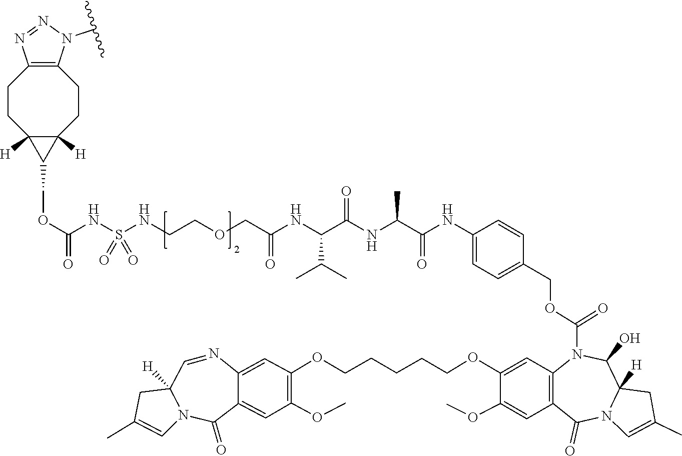 | B9 |

55. The conjugate according to any one of statements 2 to 54, wherein p is 1 to 4.
56. The conjugate according to statement 55, wherein p is 2 to 4.
57. The conjugate according to statement 55, wherein p is 1 to 3.
58. The conjugate according to statement 55, wherein p is 2.
59. The conjugate according to any one of statements 1 to 58, wherein the antibody comprises a VH domain having a VH CDR3 with the amino acid sequence of SEQ ID NO. 5.
60. The conjugate according to any one of statements 1 to 59, wherein the antibody comprises a VH domain comprising a VH CDR2 with the amino acid sequence of SEQ ID NO. 4, and/or a VH CDR1 with the amino acid sequence of SEQ ID NO. 3.
61. The conjugate according to any one of statements 1 to 60, wherein the antibody comprises a VH domain comprising a VH CDR3 with the amino acid sequence of SEQ ID NO. 5., a VH CDR2 with the amino acid sequence of SEQ ID NO. 4, and a VH CDR1 with the amino acid sequence of SEQ ID NO. 3.
62. The conjugate according to any one of statements 1 to 60, wherein the antibody comprises a VH domain comprising a VH CDR1, a VH CDR2, and a VH CDR3, and wherein the antibody comprises the CDR sequences of the VH domain having the sequence of SEQ ID NO: 1.
63. The conjugate according to any one of statements 1 to 62, wherein the antibody comprises a VH domain having the sequence of SEQ ID NO. 1.
64. The conjugate according to any one of statements 1 to 63, wherein the antibody comprises a VL domain having a VL CDR3 with the amino acid sequence of SEQ ID NO. 8.
65. The conjugate according to any one of statements 1 to 64, wherein the antibody comprises a VL domain comprising a VL CDR2 with the amino acid sequence of SEQ ID NO. 7, and/or a VL CDR1 with the amino acid sequence of SEQ ID NO. 6.
66. The conjugate according to any one of statements 1 to 65, wherein the antibody comprises a VL domain comprising a VL CDR3 with the amino acid sequence of SEQ ID NO. 8., a VL CDR2 with the amino acid sequence of SEQ ID NO. 7, and a VL CDR1 with the amino acid sequence of SEQ ID NO. 6.
67. The conjugate according to any one of statements 1 to 63, wherein the antibody comprises a VL domain comprising a VL CDR1, a VL CDR2, and a VL CDR3, and wherein the antibody comprises the CDR sequences of the VL domain having the sequence of SEQ ID NO: 2.
68. The conjugate according to any one of statements 1 to 67, wherein the antibody comprises a VL domain having the sequence of SEQ ID NO. 2.
69. The conjugate according to any one of statements 1 to 58, wherein the antibody comprises a VH domain having a VH CDR3 with the amino acid sequence of SEQ ID NO. 15.
70. The conjugate according to any one of statements 1 to 58 or 69, wherein the antibody comprises a VH domain comprising a VH CDR2 with the amino acid sequence of SEQ ID NO. 14, and/or a VH CDR1 with the amino acid sequence of SEQ ID NO. 13.
71. The conjugate according to any one of statements 1 to 58 or 69 to 70, wherein the antibody comprises a VH domain comprising a VH CDR3 with the amino acid sequence of SEQ ID NO. 15, a VH CDR2 with the amino acid sequence of SEQ ID NO. 14, and a VH CDR1 with the amino acid sequence of SEQ ID NO. 13.
72. The conjugate according to any one of statements 1 to 58, wherein the antibody comprises a VH domain comprising a VH CDR1, a VH CDR2, and a VH CDR3, and wherein the antibody comprises the CDR sequences of the VH domain having the sequence of SEQ ID NO: 11.
73. The conjugate according to any one of statements 1 to 58 or 69 to 72, wherein the antibody comprises a VH domain having the sequence of SEQ ID NO. 11.
74. The conjugate according to any one of statements 1 to 58 or 69 to 73, wherein the antibody comprises a VL domain having a VL CDR3 with the amino acid sequence of SEQ ID NO. 18.
75. The conjugate according to any one of statements 1 to 58 or 69 to 74, wherein the antibody comprises a VL domain comprising a VL CDR2 with the amino acid sequence of SEQ ID NO. 17, and/or a VL CDR1 with the amino acid sequence of SEQ ID NO. 16.
76. The conjugate according to any one of statements 1 to 58 or 69 to 75, wherein the antibody comprises a VL domain comprising a VL CDR3 with the amino acid sequence of SEQ ID NO. 18., a VL CDR2 with the amino acid sequence of SEQ ID NO. 17, and a VL CDR1 with the amino acid sequence of SEQ ID NO. 16.
77. The conjugate according to any one of statements 1 to 58 or 69 to 73, wherein the antibody comprises a VL domain comprising a VL CDR1, a VL CDR2, and a VL CDR3, and wherein the antibody comprises the CDR sequences of the VL domain having the sequence of SEQ ID NO: 12.
78. The conjugate according to any one of statements 1 to 58 or 69 to 77, wherein the antibody comprises a VL domain having the sequence of SEQ ID NO. 12.
79. The conjugate according to any one of statements 1 to 58, wherein the antibody comprises a first antigen binding domain and a second antigen binding domain, wherein:
(iii) the first antigen binding domain comprises a VH domain according to any one of statements 69 to 73, and a VL domain according to any one of statements 50 to 54; and
(iv) the second antigen binding domain comprises a VH domain according to any one of statements 69 to 73, and a VL domain according to any one of statements 60 to 64.
   80. The conjugate according to any one of statements 1 to 79, wherein the antibody in an intact antibody.
   81. The conjugate according to any one of statements 1 to 80, wherein the antibody is humanised, deimmunised or resurfaced.
   82. The conjugate according to any one of statements 1 to 80, wherein said antibody is non-human.
   83. The conjugate according to statement 82 wherein said antibody is a rat antibody.
   84. The conjugate according to statement 83 wherein said antibody is a rat IgG2b.
   85. The conjugate according to any one of statements 1 to 58, wherein the antibody comprises a heavy chain having the sequence of SEQ ID NO. 9.
   86. The conjugate according to any one of statements 1 to 58 and 85, wherein the antibody comprises a light chain having the sequence of SEQ ID NO. 10.
   87. The conjugate according to any one of statements 1 to 58, wherein the antibody comprises a heavy chain having the sequence of SEQ ID NO. 19.
   88. The conjugate according to any one of statements 1 to 58 and 87, wherein the antibody comprises a light chain having the sequence of SEQ ID NO. 20.
   89. The conjugate according to any one of statements 1 to 58, wherein the antibody is the YTH 24.5 antibody as disclosed in WO1995/013093.
   90. The conjugate according to any one of statements 1 to 58, wherein the antibody is the YTH 54.12 antibody as disclosed in WO1995/013093.
   91. A pharmaceutical composition comprising the conjugate of any one of statements 1 to 90 and, optionally, a pharmaceutically acceptable diluent, carrier or excipient.
   92. A pharmaceutical composition comprising a first conjugate, a second conjugate, and optionally a pharmaceutically acceptable diluent, carrier or excipient, wherein:
(i) the first conjugate is a conjugate according to any one of statements 59 to 68, 85, 86, or 89; and
(ii) the second conjugate is a conjugate according to any one of statements 69 to 78, 87, 88, or 90.
   93. The pharmaceutical composition of statement 92, wherein:
(i) the first conjugate is a conjugate according to statement 86; and
(ii) the second conjugate is a conjugate according to statement 88.
   94. The pharmaceutical composition of any one of statements 91 to 93, further comprising a therapeutically effective amount of a chemotherapeutic agent.
   95. The conjugate according to any one of statements 1 to 90, or the pharmaceutical composition of any one of statements 91 to 94, for use in therapy.
   96. A method of treating a proliferative disease, the method comprising administering an effective amount of the conjugate according to any one of statements 1 to 90, or the pharmaceutical composition of any one of statements 91 to 94, to a subject.
   97. The method according to statement 96, wherein the proliferative disease is cancer.
   98. The method according to statement 97, wherein the cancer is a haematological cancer.
   99. The method according to statement 98, wherein the haematological cancer is selected from the group consisting of: acute myeloid leukaemia, myelodysplasia, acute lymphoblastic leukaemia, chronic myeloid leukaemia, chronic lymphocytic leukaemia, myeloproliferative disorders, myelodysplasia, non-Hodgkins lymphoma, and Hodgkins disease and multiple myeloma.
   100. The method according to statement 97, wherein the cancer is a cancer selected from the group consisting of: Hepatocellular carcinoma, hepatoblastoma, non small cell lung cancer, small cell lung cancer, colon cancer, breast cancer, gastric cancer, pancreatic cancer, neuroblastoma, adrenal gland cancer, pheochromocytoma, paraganglioma, thyroid medullary carcinoma, skeletal muscle cancer, liposarcoma, glioma, Wilms tumor, neuroendocrine tumors, Acute Myeloid Leukemia, and Myelodysplastic syndrome.
   101. A method of preparing a subject for transplantation of haematopoietic stem cells, the method comprising administering to the subject the conjugate according to any one of statements 1 to 90 or the pharmaceutical composition of any one of statements 91 to 94.
   102. The method of statement 101, wherein the preparing for transplantation of haematopoietic stem cells comprises conditioning the subject for engraftment of haematopoietic stem cells.
   103. The method of either one of statements 101 or 102, wherein the method comprises selectively depleting or ablating an endogenous hematopoietic stem cell (HSC) or progenitor cell population in a target tissue.
   104. The method of any one of statements 101 or 103, wherein the haematopoietic stem cells are allogeneic.
   105. The method of statement 104, wherein the transplantation of haematopoietic stem cells is for treating a proliferative disease, optionally wherein the proliferative disorder is selected from the group consisting of: acute myeloid leukaemia, myelodysplasia, acute lymphoblastic leukaemia, chronic myeloid leukaemia, chronic lymphocytic leukaemia, myeloproliferative disorders, myeloma, myelodysplasia, non-Hodgkins lymphoma, and Hodgkins disease and multiple myeloma.
   106. The method according to statement 105 wherein CD45 is present on proliferative tumour cells in the subject.
   107. The method of statement 104, wherein the transplantation of haematopoietic stem cells is for treating a non-proliferative disease, optionally wherein the non-proliferative disease is selected from the group consisting of: severe aplastic anaemia, a bone marrow failure disorder, a primary immunodeficiency, a haemoglobinopathy, primary haemophagocytic lymphohistiocytosis and a genetic metabolic disease.
   108. The method of either one of statements 104 or 107, wherein the transplantation of haematopoietic stem cells is for treating:
(i) a bone marrow failure disorder selected from severe aplastic anaemia, Fanconi anaemia, dyskeratosis congenital, Schwachmann-Diamond Syndrome, severe congenital neutropaenia, Diamond-Blackfan anaemia;
(ii) a primary immunodeficiency selected from SCID, chronic granulomatous disease, Wiskott-Aldrich syndrome, CD40 ligand deficiency, XLP, MHC Class II deficiency, and primary haemophagocytic lymphohistiocytosis;
(iii) a haemoglobinopathy selected from sickle cell disease, β-thalassaemia major; or
(iv) a genetic metabolic disease selected from Hurler syndrome, X-linked adrenoleukodystrophy, alpha mannosidosis, and osteopetrosis.
   109. The method of any one of statements 101 or 103, wherein the haematopoietic stem cells are autologous.
   110. The method of statement 109, wherein the transplantation of haematopoietic stem cells is for treating a proliferative disease, optionally wherein the proliferative disease is selected from the group consisting of: multiple myeloma, non-Hodgkin's lymphoma, Hodgkin's disease.
   111. The method of statement 109, wherein the transplantation of haematopoietic stem cells is for treating an autoimmune disease, optionally wherein the autoimmune disease is selected from the group consisting of: multiple sclerosis, systemic sclerosis, juvenile inflammatory arthritis, and systemic lupus erythematosus.
   112. The method of any one of statements 101 or 103, wherein the haematopoietic stem cells are genetically-modified autologous haematopoietic stem cells.
   113. The method of any one of statements 101 to 103, or 112, wherein said transplantation of haematopoietic stem cells is for gene therapy.
   114. The method of statement 113, wherein the gene therapy is for treating a genetic haematological disease, a primary immunodeficiency, or a genetic metabolic disorder.
   115. The method of either one of statements 113 or 114, wherein the gene therapy is for treating:
(i) a genetic haematological disease or disorder selected from a transfusion dependent haemoglobinopathy, sickle cell disease, β-thalassaemia major, and Fanconi anaemia;
(ii) a primary immunodeficiency selected from SCID, chronic granulomatous disease, primary haemophagocytic lymphohistiocytosis, and Wiskott-Aldrich syndrome; or
(iii) a genetic metabolic disorder selected from Hurler's syndrome, Sanfilippo disease, X-adrenoleukodystrophy, and metachromatic leukodystrophy.
   116. A method of engrafting stem cells in a subject, the method comprising:
(a) administering to the subject an effective amount of the conjugate according to any one of statements 1 to 90 or the pharmaceutical composition of any one of statements 91 to 94; and
(b) administering a stem cell population to the target tissue of the subject, wherein the administered stem cell population engrafts in the target tissue of the subject.
   117. A method of selectively depleting or ablating an endogenous hematopoietic stem cell (HSC) or progenitor cell population in a target tissue of a subject, the method comprising administering to the subject an effective amount of the conjugate according to any one of statements 1 to 90 or the pharmaceutical composition of any one of statements 91 to 94.
   118. The method of any one of statements 103 to 117, wherein the target tissue comprises bone marrow tissue.
   119. The method of any one of statements 101 to 118, wherein the method achieves at least about 90% engraftment of the stem cell population.
   120. The method of any one of statements 101 to 118, wherein the method achieves at least about 20% donor chimerism in the target tissue four months post-administration of the stem cell population to the subject.
   121. The method of any one of statements 96 to 120, wherein the subject is a mammal.
   122. The method of statement 121, wherein the subject is a human.
   123. The method of any one of statements 96 to 122, wherein the subject is immunocompetent.
   124. The method of any one of statements 96 to 123, wherein the step of administering to the subject an effective amount of the conjugate according to any one of statements 1 to 76 comprises administering to the subject:
(i) a first conjugate according to any one of statements 1 to 90; in combination with
(ii) a second, different conjugate according to any one of statements 1 to 90.
   125. The method of statement 124, wherein:
(i) the first conjugate is a conjugate according to any one of statements 59 to 68, 85, 86, or 89; and
(ii) the second conjugate is a conjugate according to any one of statements 69 to 78, 87, 88, or 90.
   126. The method of statement 125, wherein:
(iii) the first conjugate is a conjugate according to statement 86; and
(iv) the second conjugate is a conjugate according to statement 88.
   127. A conjugate according to any one of statements 1 to 90 or the pharmaceutical composition of any one of statements 91 to 94 for use in a method according to any one of statements 82 to 112.
   128. Use of a conjugate according to any one of statements 1 to 90 or the pharmaceutical composition of any one of statements 91 to 94 in the manufacture of a medicament for use in a method according to any one of statements 96 to 126.

### EXAMPLES

### Preparation of ADCs

Antibody-Drug Conjugate YTH24.5-rlgG2-B1

A 1 mM solution of Tris(2-carboxyethyl)phosphine (TCEP) in phosphate-buffered saline pH 7.4 (PBS) was added (2.9 molar equivalent/antibody, 0.51 micromoles, 509 μL) to a ˜26 mL solution of antibody (26.3 mg, 175 nanomoles) in reduction buffer containing PBS and 2.4 mM ethylenediaminetetraacetic acid (EDTA; actual 2.4 mM but desired concentration 1 mM) and a final antibody concentration of 1.0 mg/mL. The reduction mixture was heated at +37° C. for 2 hours in an incubated orbital shaker with gentle (60 rpm) shaking. After 5 min cooling down the reduction mixture to room temperature in a water bath, DMSO (2.5 mL) was added to this reduced antibody solution (26.3 mg, 175 nanomoles) for a 10% (v/v) final DMSO concentration and a final antibody concentration of 1.0 mg/mL, and cooled to room temperature in a water bath for a further 5 min.

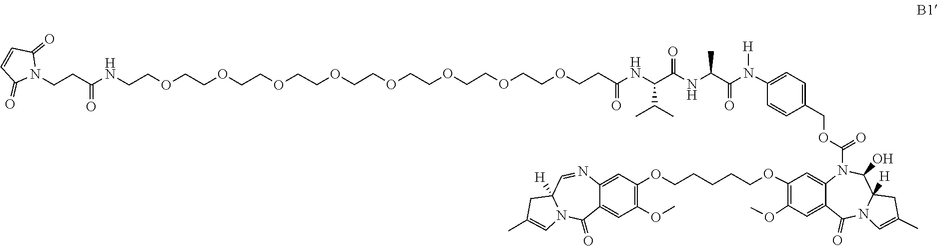 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)N=C[C@@H]1CC(C)=CN1C2=O]

B1′ was then added as a 10 mM stock solution in DMSO (8 molar equivalent/antibody, 1.40 micromoles, 140 μL). The solution was mixed for 1 hour 35 min at room temperature, then the conjugation was quenched by addition of N-acetyl-L-cysteine (7.01 micromoles, 70.1 μL at 100 mM). After 15 min approximately ⅓ of the reaction mixture was purified on an AKTA™ Start FPLC using a GE Healthcare HiLoad™ 26/600 column packed with Superdex 200 PG, eluting with 2.6 mL/min PBS. The rest of the reaction mixture was left overnight in the fridge and purified in two approximately equal portions the following day by preparative SEC-FPLC as above. Fractions corresponding to the conjugate monomer peak were pooled, concentrated using a 15 mL Amicon Ultracell 30 kDa MWCO spin filter, sterile-filtered and analysed.

UHPLC analysis on a Shimadzu Prominence system using a Thermo Scientific MAbPac 50 mm×2.1 mm column eluting with a gradient of water and acetonitrile on a reduced sample of the conjugate at 214 nm and 330 nm (B1′ specific) shows a mixture of unconjugated light chains, light chains attached to a single molecule of B1, unconjugated heavy chains and heavy chains attached to up to five molecules of B1, consistent with a drug-per-antibody ratio (DAR) of 1.94 molecules of B1 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of the conjugate at 280 nm shows a monomer purity of >95%. UHPLC SEC analysis gives a concentration of final conjugate at 1.33 mg/mL in 7.0 mL, obtained mass of conjugate is 9.3 mg (35% yield).

Antibody-Drug Conjugate YTH54.12-rlgG2-B1

A 1 mM solution of Tris(2-carboxyethyl)phosphine (TCEP) in phosphate-buffered saline pH 7.4 (PBS) was added (1.6 molar equivalent/antibody, 257 nanomoles, 257 μL) to a ˜23.5 mL solution of antibody (23.8 mg, 159 nanomoles) in reduction buffer containing PBS and 1 mM ethylenediaminetetraacetic acid (EDTA) and a final antibody concentration of 1.0 mg/mL. The reduction mixture was heated at +37° C. for 2 hours in an incubated orbital shaker with gentle (60 rpm) shaking. After 5 min cooling down the reduction mixture to room temperature in a water bath, DMSO (2.25 mL) was added to this reduced antibody solution (23.8 mg, 159 nanomoles) for a 10% (v/v) final DMSO concentration and a final antibody concentration of 1.0 mg/mL, and cooled to room temperature in a water bath for a further 5 min.

B1′ was then added as a 10 mM stock solution in DMSO (8 molar equivalent/antibody, 1.27 micromoles, 127 μL). The solution was mixed for 1 hour 35 min at room temperature, then the conjugation was quenched by addition of N-acetyl-L-cysteine (6.35 micromoles, 63.5 μL at 100 mM). After 15 min approximately ⅓ of the reaction mixture was purified on an AKTA™ Start FPLC using a GE Healthcare HiLoad™ 26/600 column packed with Superdex 200 PG, eluting with 2.6 mL/min PBS. The rest of the reaction mixture was left overnight in the fridge and purified in two approximately equal portions the following day by preparative SEC-FPLC as above. Fractions corresponding to the conjugate monomer peak were pooled, concentrated using a 30 kDa MWCO spin filter, sterile-filtered and analysed.

UHPLC analysis on a Shimadzu Prominence system using a Thermo Scientific MAbPac 50 mm×2.1 mm column eluting with a gradient of water and acetonitrile on a reduced sample of the conjugate at 214 nm and 330 nm (B1′ specific) shows a mixture of unconjugated light chains, light chains attached to a single molecule of B1, unconjugated heavy chains and heavy chains attached to up to five molecules of B1, consistent with a drug-per-antibody ratio (DAR) of 1.90 molecules of B1 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of the conjugate at 280 nm shows a monomer purity of 97%. UHPLC SEC analysis gives a concentration of final conjugate at 1.83 mg/mL in 7.5 mL, obtained mass of the conjugate is 13.7 mg (58% yield).

### Example 1. Specific Expression of CD45 on Human Haemopoietic Tissue

### Materials and Methods

Human CD34+ cells were purified from mobilized peripheral blood or umbilical cord blood using magnetic activated cell sorting using CD34 Microbeads (Miltenyi Biotec) according to manufacturer's protocol. CD45 levels were determined using anti-human CD45-PE (clone H130) (Biolegend) and QuantiBRITE PE beads (BD) following manufacturer's instructions. Northern blot analysis was carried using standard molecular biology techniques.

### Results

See FIG. 1.

Approximately 25,000 and 10,000 molecules of CD45 were detected on the surface of bulk CD34+ cells purified from mobilized peripheral blood and umbilical cord blood respectively.

CD45 mRNA expression was restricted to thymus spleen and peripheral blood with low or absent expression in brain, heart, colon, skeletal muscle, kidney, liver and small intestine.

### Conclusions.

These data show that there are significant levels of cell surface CD45 on normal bulk CD34+ cells from mobilised peripheral blood and umbilical cord blood. The mRNA expression studies showed that CD45 is specific and is expressed at high levels in haemopoietic tissues such as thymus, spleen and blood and absent in non-haemopoietic tissues. Low levels of CD45 RNA in heart and liver is due to the presence of contaminating hemopoietic cells. These data indicated that antibodies targeting CD45 will specifically target CD45 expressing cells in haemopoietic tissues and spare non-haemopoietic tissue.

### Example 2. Anti-Human CD45 Clones YTH24.5 and YTH54.12 Demonstrate Synergistic Lytic Activity in Presence of Complement on Cell Lines

### Materials and Methods

1×10⁶ human CD45+ OCIM1 cells in 100 uL of complete medium (RPMI 1640 plus 10% fetal calf serum) were radiolabelled with 100 μCi ⁵¹Cr sodium chromate for 1 h at 37° C. followed by three washes with complete medium. Labelled cells were incubated at 5×10⁴/mL in triplicate wells of a 96 well plate in presence of YTH24.5 alone, YTH54.12 alone or with both antibodies for 10 mins at room temperature before of addition of baby rabbit serum at a final concentration of 10%. A final concentration of 5% Triton X-100 was added to control wells of cells to determined maximum lysis. 50 uL of supernatant from each well was transferred into a white-walled plates containing 150 uL scintillation liquid in each well. Plates were read in a MicroBeta counter (Trilux) after overnight incubation. The percentage of specific lysis was calculated using the standard formula [(experimental−spontaneous release)/(maximum load-spontaneous release)×100] and expressed as the mean of triplicate samples.

### Results

See FIG. 2.

YTH24.5 showed lytic activity on OCIM1 cells at 10 ug/ml whereas YTH54.12 demonstrated no lytic activity on OCIM1 cells at 10 ug/ml.

YTH24.5 and YTH54.12 at 1:1 ratio demonstrated maximum lysis of cells at 0.2 ug/mL.

### Conclusions

These data show that YTH24.5 has greater lytic activity than YTH54.12 against CD45+ cell lines in CDC assays at 10 ug/mL. However, they induced synergistic lysis when used at 1:1 ratio at 0.2 ug/mL. It is known that YTH24.5 and YTH54.12 recognised different epitopes.

### Example 3. Anti-Human CD45 Antibodies Inhibit Colony Formation in Presence of Complement

### Materials and Methods

Cryopreserved purified CD34+ cells from mobilized peripheral blood from a normal donor were thawed and cultured at 0.5×10⁵/mL complete medium (RPMI 1640 plus 10% fetal calf serum) in presence of 10 ug/mL of YTH24.5 and YTH54.12 Mab or 10 ug/mL isotype control or no antibody for 2 hours in absence or presence of 10% baby rabbit complement. 2×10³ cells were transferred to 2 ml of StemMACS HSC-CFU complete with Epo (Miltenyi Biotech) and mixed well. 0.5 ml (500 cells) was plated into triplicate into wells of a 24-well plate. Colonies were enumerated between 10-14 days.

### Results

See FIG. 3.

In absence of complement, anti-human CD45 treated cells generated the same number of colonies and isotype control and no antibody controls.

In presence of complement, YTH24.5 and YTH54.12 complete abrogate colony formation.

### Conclusions

These data show that YTH24.5 and YTH54.12 are highly lytic and completely inhibit colony formation of normal human CD34+ cells purified from mobilized peripheral blood. Bulk CD34+ cells are comprised of true HSCs and more committed progenitors and express CD45 at lower levels than mature haemopoietic cells and human cell lines (data not shown). Therefore despite lower levels of CD45 on CD34+ cells, YTH24.5 and YTH54.12 retain their high lytic activity in in vitro clonogenic assays.

### Example 4. Treatment of Human CD34+ Cells with Anti-Human CD45 MAbs and Complement Prevents Engraftment Cells in Immunodeficient NSG Mice

### Materials and Methods

Cryopreserved human CD34+ cells were thawed and cultured with 10 ug/mL rat IgG2b isotype antibody or a mixture of 10 ug/ml of each anti-human CD45 MAb (YTH24.5 and YTH54.12) for 10 minutes before addition of 10% baby rabbit serum. After 2 hours incubation with serum, cells were washed with PBS before intravenous injection into NSG mice which had received 2.5 Gy dose in a ¹³⁷Cs irradiator. Mice were culled at 15 to 16 weeks post-transplant and haemopoietic tissues were analysed for human cell engraftment using flow cytometry.

### Results

See FIG. 4.

Isotype control plus complement treated cells resulted in approximately 50% human CD45+ cell engraftment in the bone marrow at 15/16 weeks post-transplant.

CD34+ cells ex vivo treated in presence of YTH24.5+ YTH54.12+ complement did not engraft in the bone marrow of NSG mice at 15/16 weeks post-transplant.

### Conclusions

YTH24.5 and YTH54.12 in presence of complement were highly effective at preventing the engraftment of human CD34+ cells in NSG mice whereas isotype control Mab in presence of complement did not inhibit human cell engraftment. The cell killing ability of these antibodies may be enhanced through the generation of ADCs using YTH24.5 and/or YTH54.12 to deliver a cytotoxic agent to antibody bound cells.

### Example 5. Anti-Human CD45 Immunotoxins Specifically Kill Human CD45+ Cells

### Material and Methods

YTH24.5, YTH54.12 and Isotype control antibodies were incubated at 4× final concentration with or without an equal volume of 18 mM (4×) anti-rat IgG-ZAP (saporin conjugate) (Advanced Targeting Systems, Carlsbad, Calif., US) in R10 medium at room temperature for 15 minutes as per manufacturer's instructions. 50 ul of the Mab/anti-rat IgG ZAP complex was mixed with 50 uL 1×10⁵/ml Jurkat cells in R10 medium (final cell density=0.5×10⁵ cells/m1) in triplicate in wells of a flat bottom 96 well plate. Cell viability was determined using PrestoBlue™ Cell Viability assay reagent after 72 h hours incubation as previously described.

### Results

See FIG. 5.

Cells treated with rat IgG2b isotype control ±anti-rat IgG saporin conjugate demonstrated high cell viability (85-100%) at all concentrations tested.

Cells treated with YTH24.5 and YTH54.12 alone showed viability (70-100%) at the concentrations tested.

Cells treated with YTH24.5+ anti-rat IgG saporin or YTH54.12+ anti-rat IgG saporin showed dose-dependent cell killing.

### Conclusions

YTH24.5 and YTH54.12 were shown to be able to internalize anti-rat IgG conjugated saporin to induce saporin-mediated cell death of CD45+ cells. This demonstrated that anti-human CD45 antibodies are internalized upon binding to CD45 and therefore be potentially be developed as ADCs.

### Example 6: Anti-CD45 PBDs (Rat IgG2b Format) Specifically Kill CD45+ Cell Lines

### Materials & Methods

OCIM1, Jurkat and Nalm6 cells were seeded at 0.5×10⁵/mL were cultured for 72 hours in presence of Mab or ADC or medium (in triplicate wells) in wells of flat bottom 96-well plates. 293T cells were seeded at 9.3×10³ cells per well in flat bottom 96-well plates and allowed to adhere the plate for up to 24 hours. 293T cells were subsequently cultured with Mab or ADC or medium (in triplicate wells) for 72 hours. PrestoBlue® Cell viability Reagent (Thermo Fisher Scientific) was added to each and incubated for up to 1.5 hours. Fluorescence intensity from each well was detected using a FLUOstar OPTIMA microplate reader (BMG Labtech). Data points are mean±s.d. for representative experiments.

The drug-linker (DL) used in the assayed ADCs is “B1” as described herein.

### Results

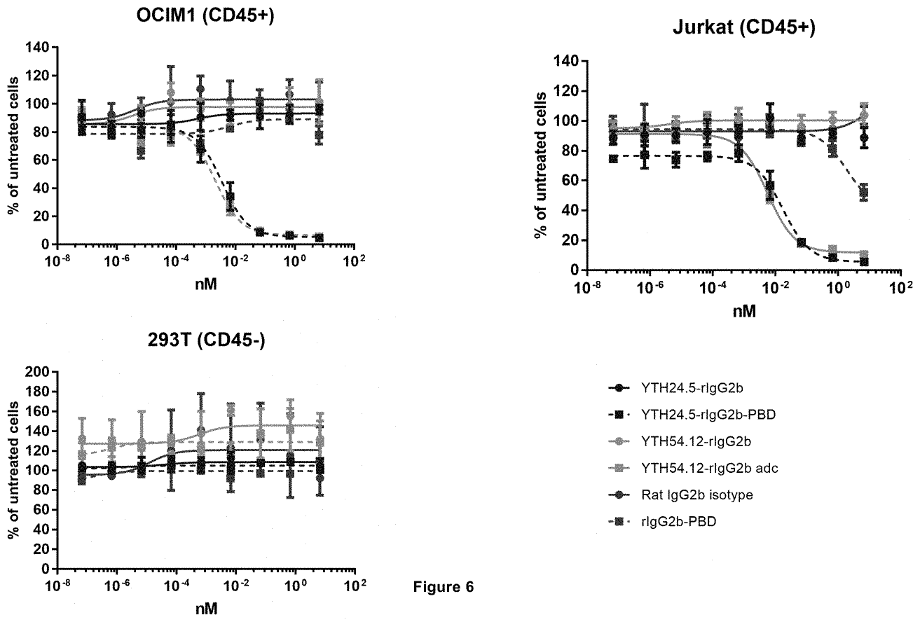

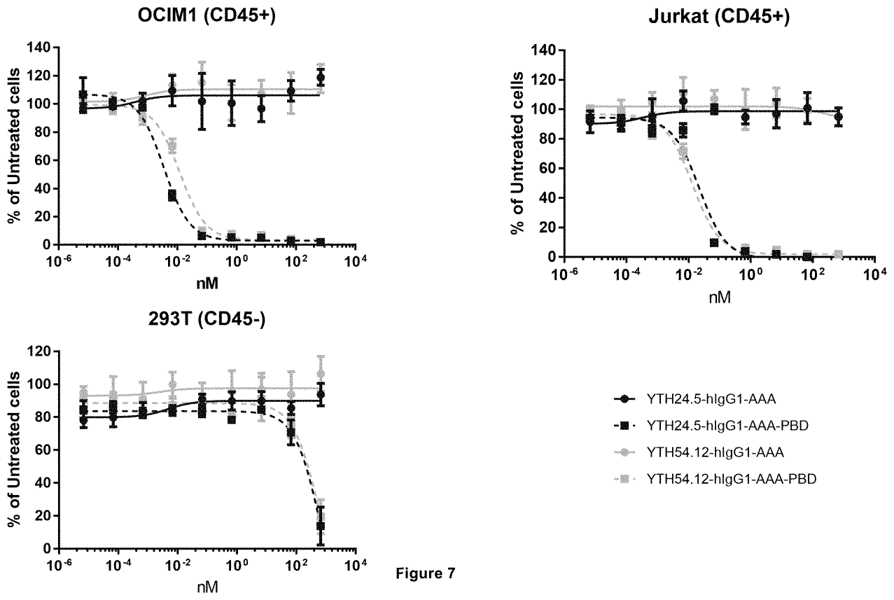

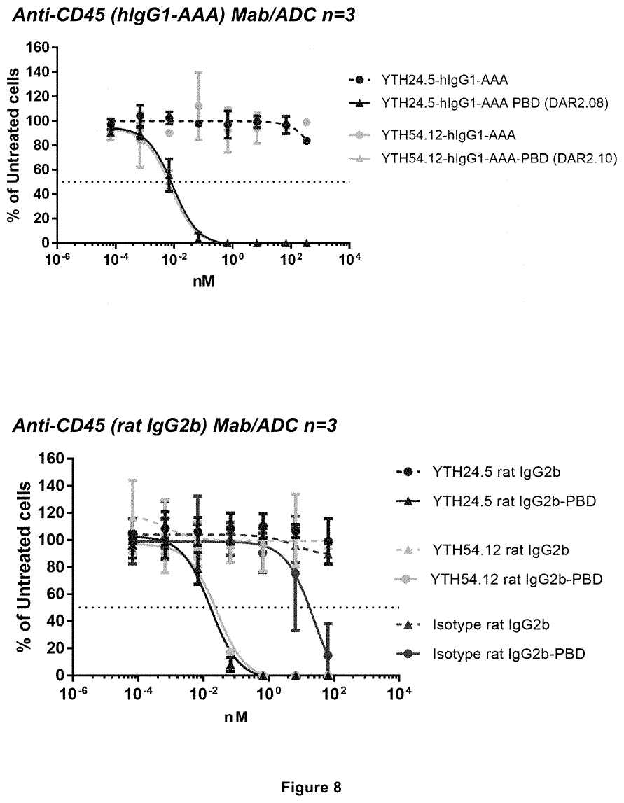

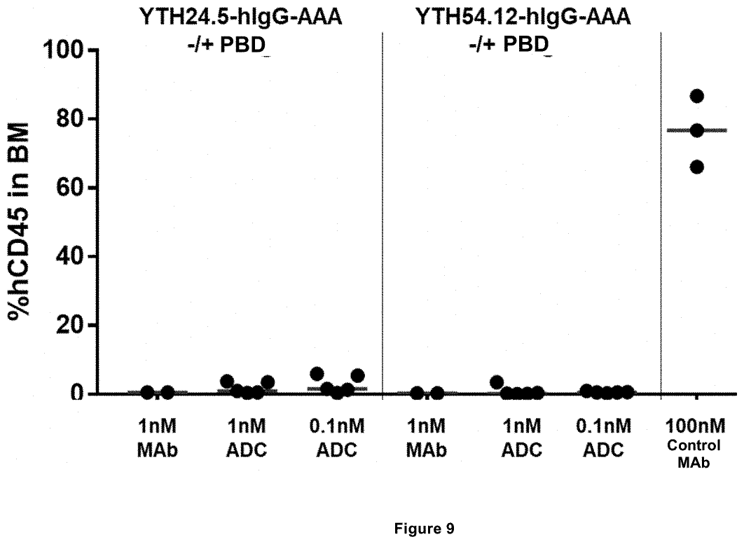

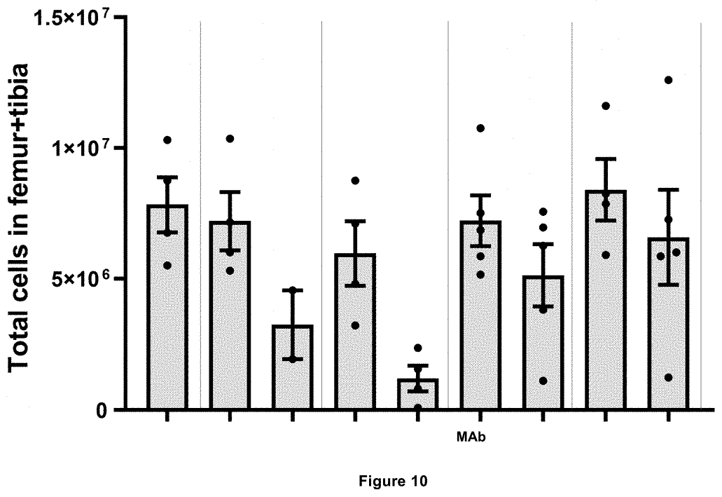

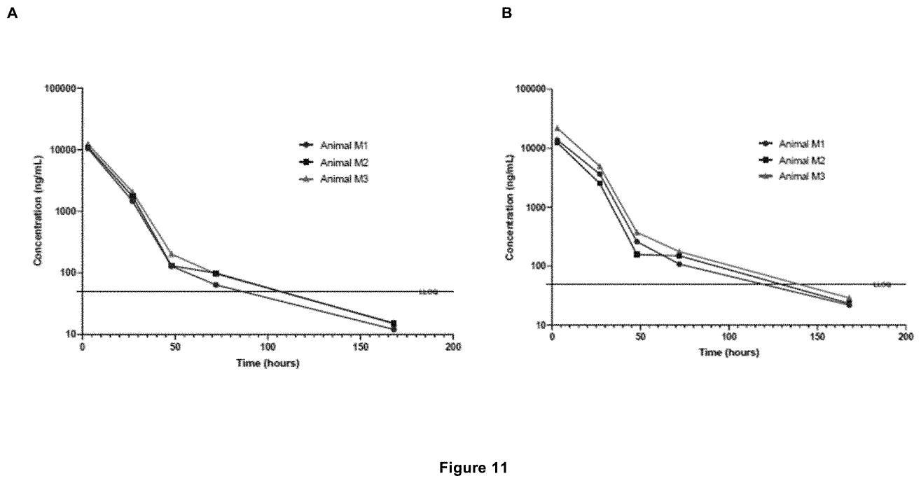

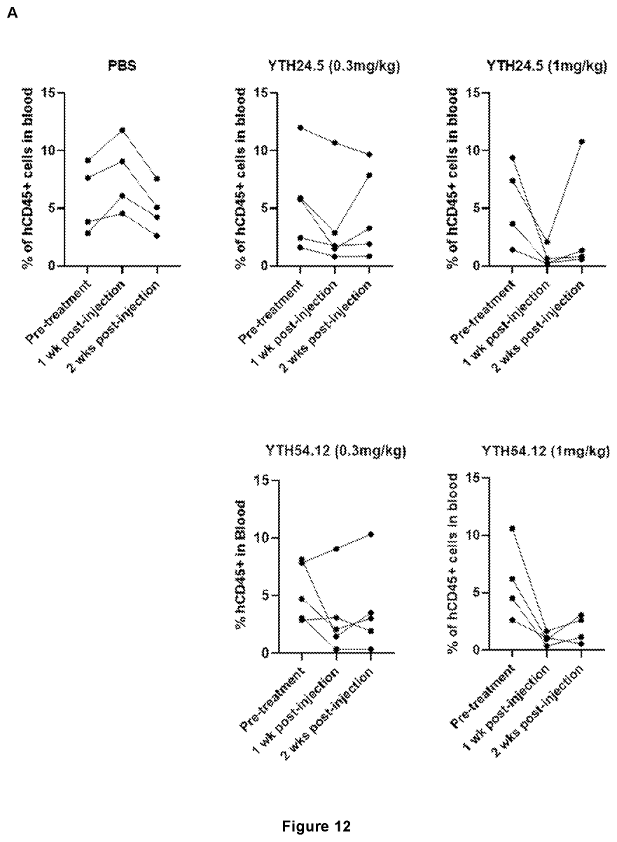

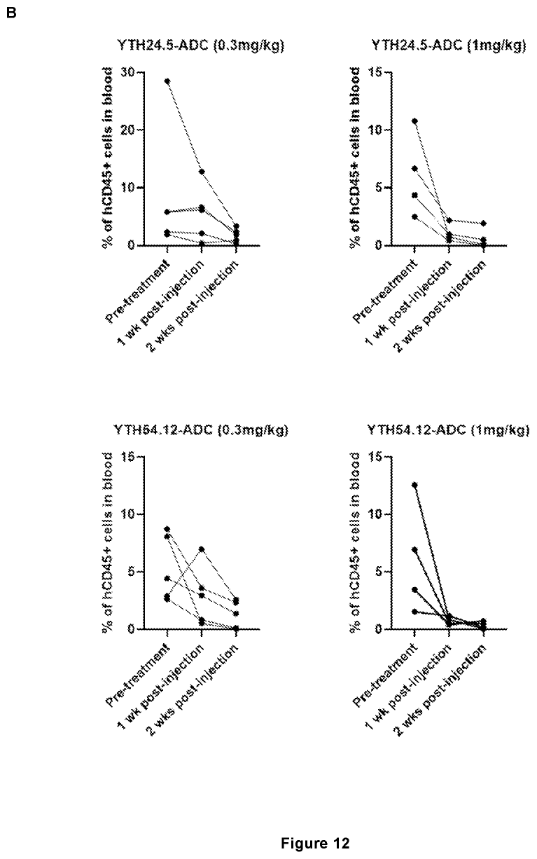

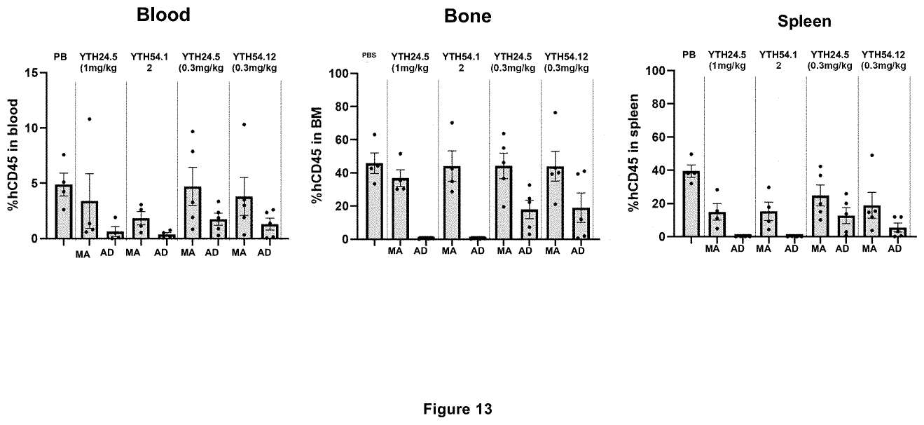

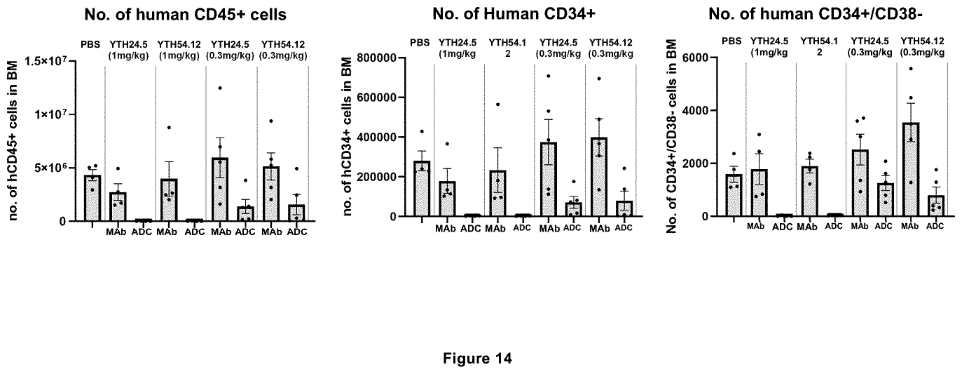

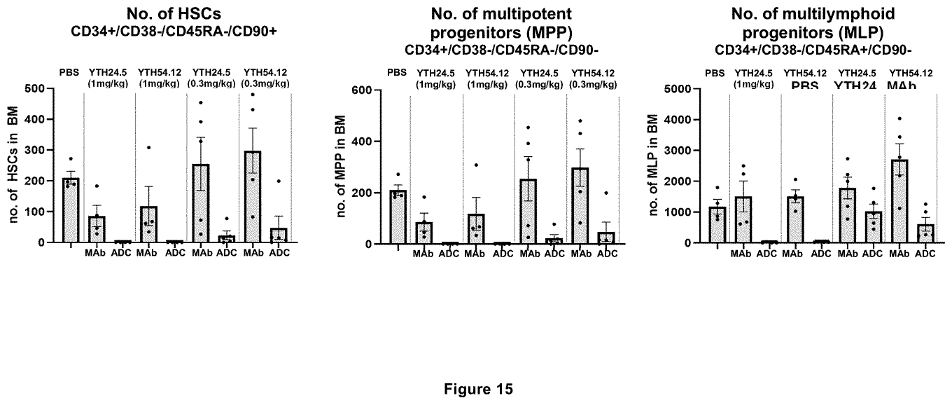

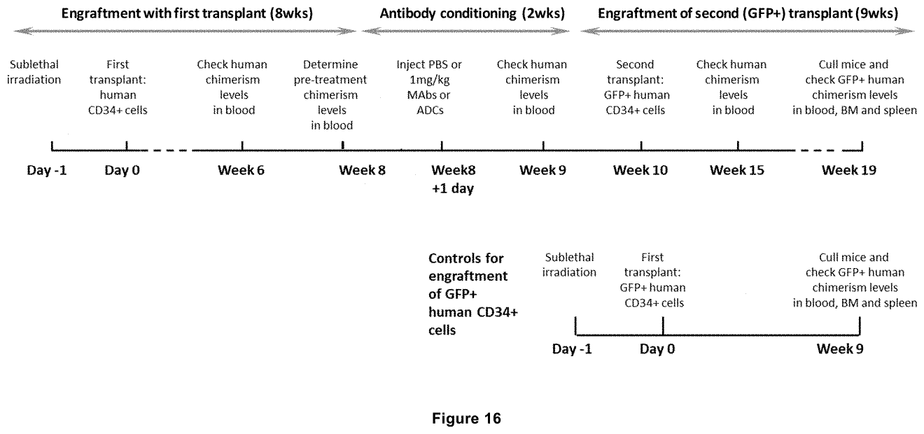

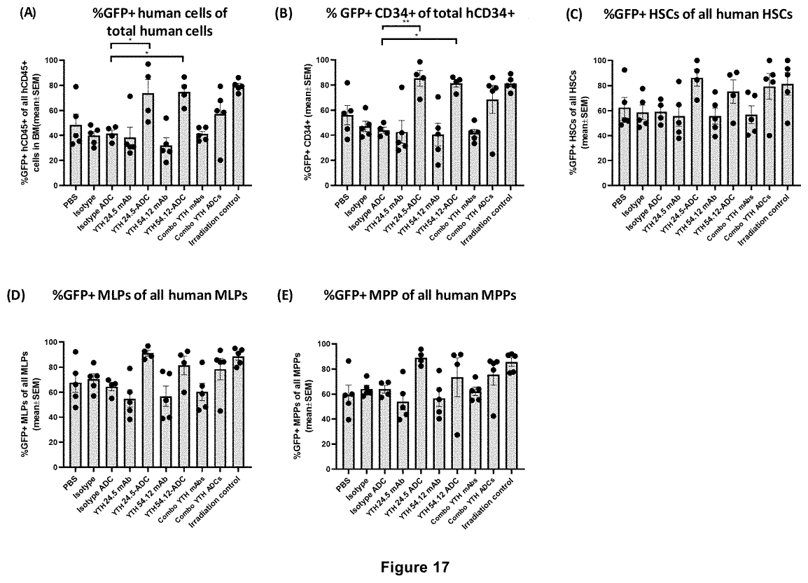

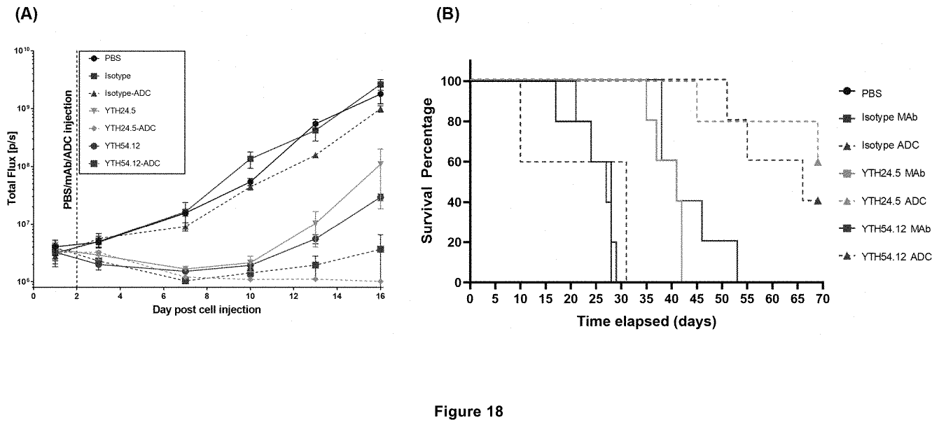

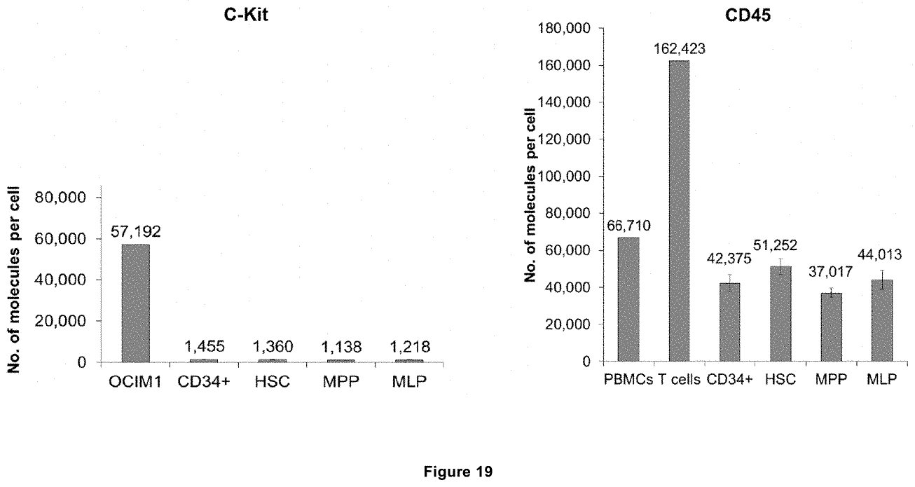

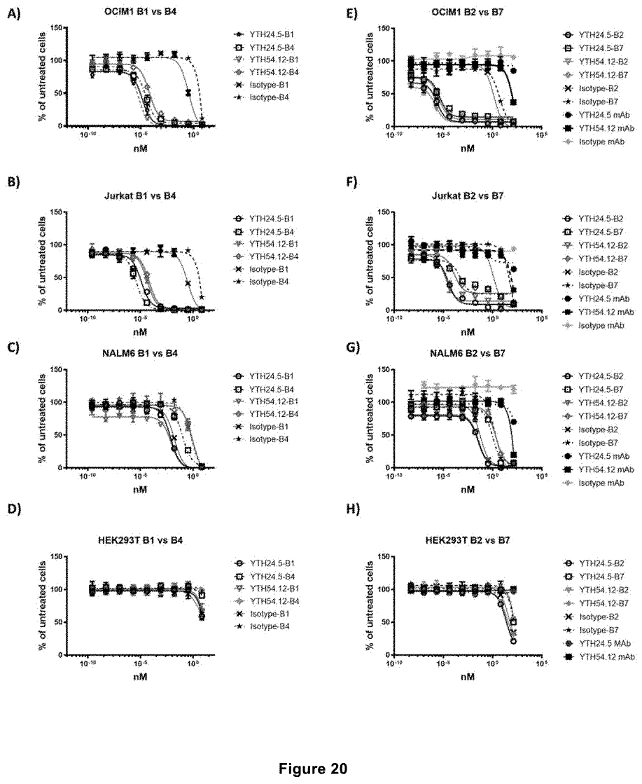

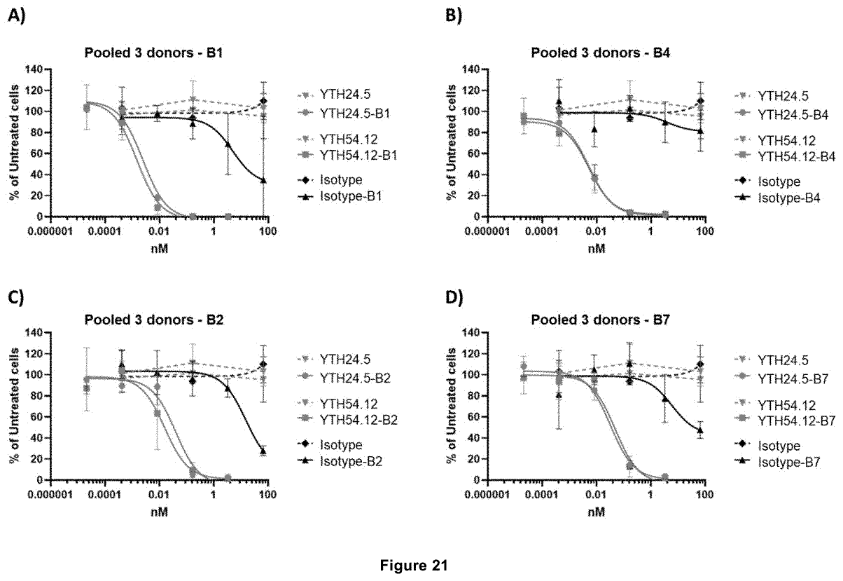

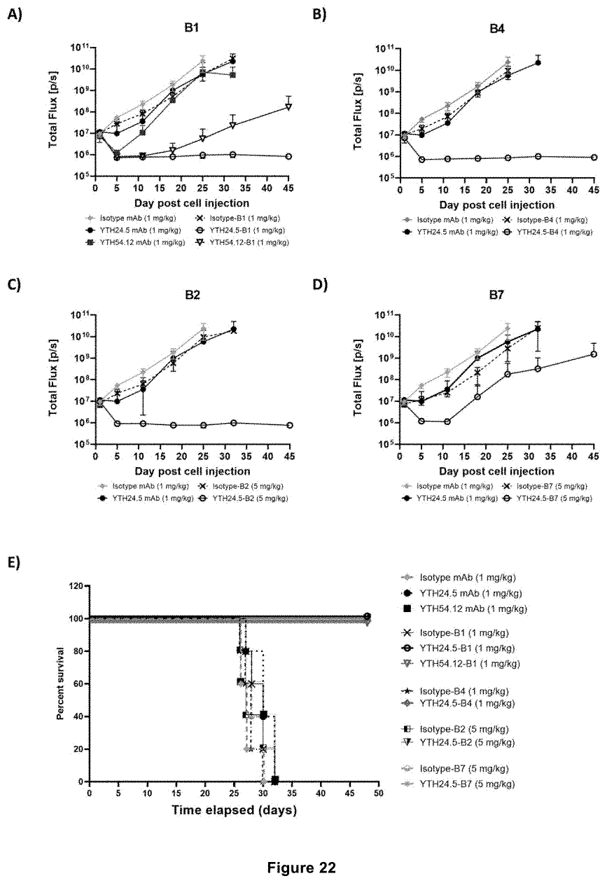

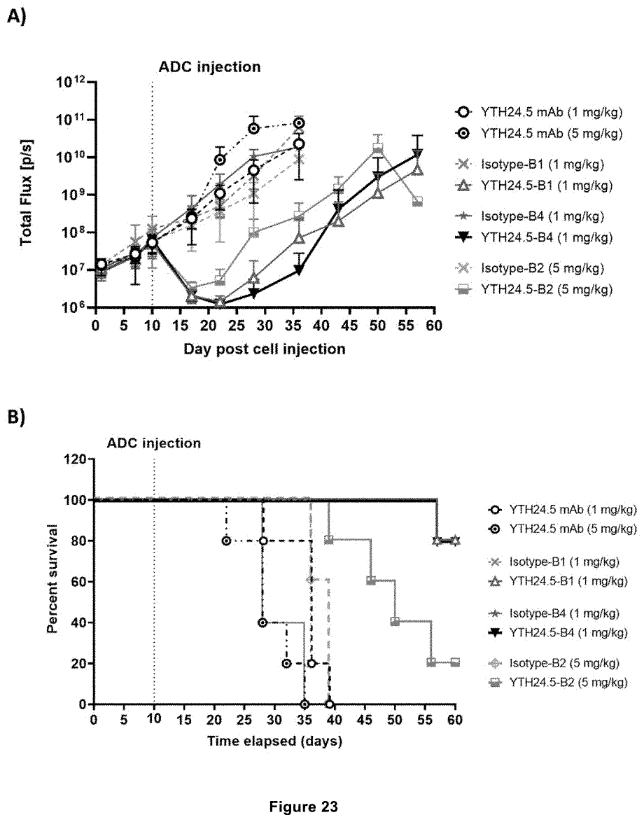

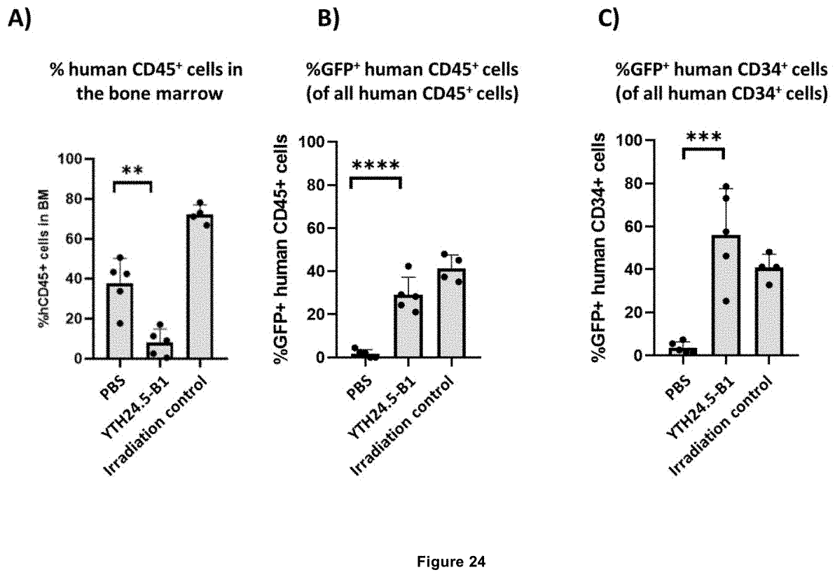

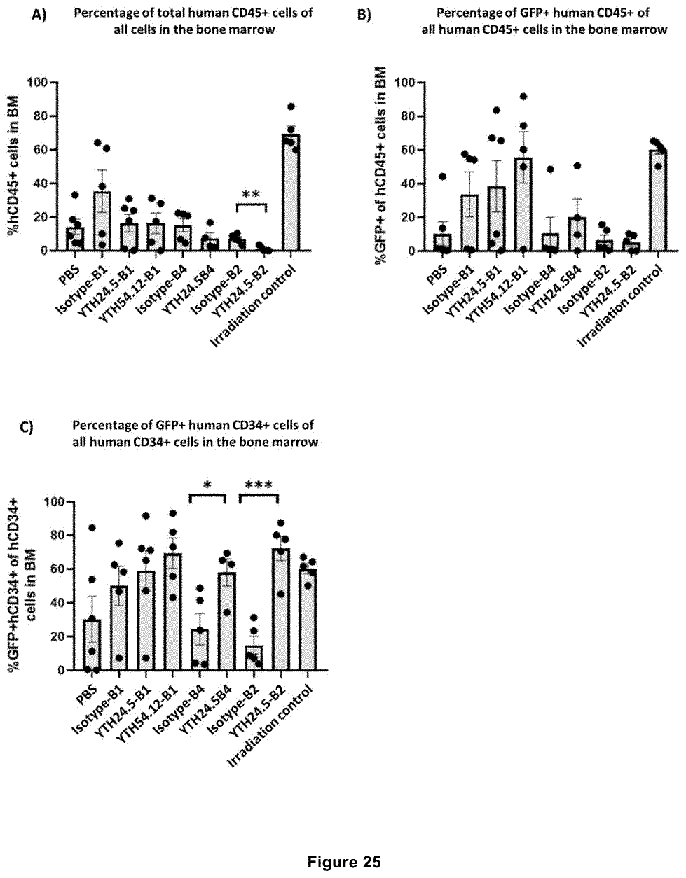

See FIG. 6 and table following Example 8.

YTH24.5-PBD and YTH54.12-PBD in rat IgG2b format specifically killed human CD45+ OCIM1 and Jurkat cells with EC50 values in the low pM range.

Isotype control-PBD had no effect on OCIM1 at the highest concentration, but some cell death was observed at the highest concentration on Jurkat cells.

CD45-293T were insensitive to the ADCs at all the concentrations tested.

The monoclonal antibodies alone did not have any adverse effect on the viability of the cells.

### Conclusions

These data demonstrate the potent and specific killing of human CD45+ cells by anti-human CD45 PBDs in a rat IgG2b format with the two anti-human CD45 clones being equally effective.

### Example 7: Anti-CD45 PBDs (Human IgG1-AAA Format) Specifically Kill CD45+ Cell Lines

### Materials & Methods

OCIM1, Jurkat and Nalm6 cells were seeded at 0.5×10⁵/mL were cultured for 72 hours in presence of Mab or ADC or medium (in triplicate wells) in wells of flat bottom 96-well plates. 293T cells were seeded at 9.3×10³ cells per well in flat bottom 96-well plates and allowed to adhere the plate for up to 24 hours. 293T cells were subsequently cultured with Mab or ADC or medium (in triplicate wells) for 72 hours. PrestoBlue® Cell viability Reagent (Thermo Fisher Scientific) was added to each and incubated for up to 1.5 hours. Fluorescence intensity from each well was detected using a FLUOstar OPTIMA microplate reader (BMG Labtech). Data points are mean±s.d. for representative experiments.

The drug-linker (DL) used in the assayed ADCs is “B1” as described herein.

### Results

See FIG. 7 and table following Example 8.

YTH24.5 and YTH54.12 in human IgG1-AAA format specifically killed human CD45+ cell lines with EC50 values in the low pM range.

CD45-ve 293T were insensitive to the ADCs with some cell kill observed at concentrations >100 nM.

### Conclusions

These data demonstrate the potent killing of human CD45+ cells by anti-human CD45 PBDs in a human IgG1-AAA format with both clones showing equivalent cell kill.

### Example 8: Anti-Human CD45 PBDs Kill Human CD34+ Clonogenic Progenitors

### Materials & Methods

Cryopreserved purified CD34+ cells from mobilized peripheral blood from three normal donors were thawed and cultured at 0.5×10⁵/mL complete medium (RPMI 1640 plus 10% fetal calf serum) in presence of Mab, ADC or medium alone for 2 hours in 96-well U-bottom plates. Cells were mixed and 40 uL was transferred to 2 ml of StemMACS HSC-CFU complete with Epo (Miltenyi Biotech) and mixed well. 0.5 ml (250 cells) was plated into triplicate into wells of a 24-well plate. Colonies were enumerated between 10-14 days. Data is mean of three donors±s.d.

The drug-linker (DL) used in the assayed ADCs is “B1” as described herein.

### Results

See FIG. 8 and table following Example 8.

YTH24.5 and YTH54.12 and in human IgG1-AAA or rat IgG2b Mab formats do not inhibit colony formation at any of the concentrations tested nor does the Isotype control (rat IgG2b format only).

YTH24.5 PBD and YTH54.12 PBD and in human IgG1-AAA showed dose-dependent inhibition of colony formation.

The Isotype control PBD (rat IgG2b format only) inhibited colony formation at the higher concentrations.

### Conclusions

Specific inhibition of colony formation is observed with EC50 in low pM range with the YTH24.5 PBD and YTH54.12 PBD in both human and rat formats whereas the isotype control PBD (rat IgG2b format) was less effective at inhibiting colony formation with nanomolar EC50 value. These data confirmed that anti-human CD45 PBDs can target and kill primary human CD45+CD34+ cells in vitro.

### Summary of Examples 6 to 8: Comparison of In Vitro EC50 Values for Anti-Human CD45 PBDs

| | | EC50 values (pM) | | | | |
|---|---|---|---|---|---|---|
| | | | | | | C'genic |
| | | Viability assays | | | | assays |
| | | (human cell lines) | | | | CD34+ |
| Antibody Drug Conjugate | | OClM1 | Jurkat | Nalm6 | 293T | PBSC |
| (ADC) | DAR | (CD45+) | (CD45+) | (CD45−) | (CD45−) | (CD45+) |
| YTH24.5-hIgG1-AAA-PBD | 2.08 | 6.8 | 23.0 | ND | 134020 | 7.9 |
| YTH24.5-rat IgG2b-PBD | 2.00 | 1.38 | 5.36 | 2832 | ND | 16.5 |
| YTH54.12-hIgG1-AAA-PBD | 2.10 | 9.7 | 18.0 | ND | 16460 | 6.4 |
| YTH54.12-rat IgG2b-PBD | 2.00 | 1.89 | 4.44 | 4226 | ND | 23.0 |
| Isotype rat IgG2b-PBD | 2.00 | 13550 | 12175 | 5737 | ND | 18040 |
| (clone RTK4530) | | | | | | |

### Conclusions

Specific killing by anti-CD45 PBD ADCs containing either of the YTH24.5 or YTH54.12 antibodies.

### Example 9: Ex Vivo Treatment of HSCs with ADCs Prior to Engraftment in NSG Mice

### Materials & Methods

Adult female NSG mice were sublethally irradiated with 2.5 Gy on the day before transplantation. Cryopreserved purified CD34+ cells from mobilized peripheral blood from a normal donor were thawed and cultured in complete medium (RPMI 1640 plus 10% fetal calf serum) in presence of Mab, ADC or medium alone for 4 hours. Cells were washed in phosphate buffered saline (PBS) and 0.5×10⁶ cells in PBS were injected intravenously into the tail vein of mice. Engraftment of transplanted human cells (hCD45+) in the bone marrow of mice was determined by flow cytometry at 8 weeks after transplantation.

The drug-linker (DL) used in the assayed ADCs is “B1” as described herein.

### Results

See FIG. 9.

YTH24.5-PBD and YTH54.12-PBD treatment of human CD34+ cells at 1 nM and 0.1 nM prior to transplantation prevented engraftment of human CD45+ cells in the bone marrow of NSG mice.

Prior treatment of human CD34+ cells with YTH24.5 and YTH54.12 naked MAbs at 1 nM and 0.1 nM also prevented engraftment of human CD45+ cells in the bone marrow of NSG mice.

A control naked MAb used at 100 nM enabled 80% human CD45+ engraftment in the bone marrow of NSG mice.

### Conclusions

Both the anti-human CD45 naked MAbs and ADCs abrogated engraftment of human CD34+ HSCs. Since the naked antibodies prevented human cell engraftment, it precluded assessment of any additive effective of the ADCs. It is proposed that the naked antibodies may deplete the antibody-bound CD34+ cells after injection into the NSG mice, possibly via an ADCP mechanism since NSG mice do not have functional complement system. It is possible that anti-human CD45 PBDs may also deplete human CD45 cells independently of the PBD payload.

### Example 10: Dosimetry in NSG Mice

### Materials & Methods

For dosimetry in non-humanised NSG mice, adult NSG mice were injected intravenously via the tail vein with unconjugated Mab or ADC up to 5 mg/kg. Mice regularly monitored over a period of 2 weeks for any signs of morbidity. Mice were culled if rapid weight loss (>15%) occurred over a period of 2 days.

For dosimetry in humanised NSG mice, adult female NSG mice were sublethally irradiated with 2.5 Gy on the day before transplantation. Cryopreserved purified CD34+ cells from mobilized peripheral blood from a normal donor were thawed and 0.5×10⁶ cells in PBS were injected intravenously into the tail vein. Human engraftment (human CD45+ cells) in the blood of all mice was determined by flow cytometry at 12 weeks post-transplant. Mice were assigned to treatment cohorts so that the median human engraftment in the blood for between cohorts was as similar as possible. Mice were treated with 0.3 mg/kg or 3 mg/kg Mab or ADC. Blood samples were taken after 7 days and analysed by flow cytometry. The bone marrow, blood and spleen of mice were analysed at 14 days post-treatment.

The drug-linker (DL) used in the assayed ADCs is “B1” as described herein.

### Results

| | | | No. of | |
|---|---|---|---|---|
| | Dose | Human- | mice | |
| Mab/ADC | injected | ised? | injected | Comment |
| YTH24.5- | 5 mg/kg | No | 2 | All alive at day 14 |
| hIgG-AAA | | | | |
| (Unconjugated) | | | | |
| YTH24.5-hIgG- | 5 mg/kg | No | 2 | 2 culled at day 6 - |
| AAA-PBD | | | | weight loss >15% |
| | 3 mg/kg | Yes | 2 | 1 culled day 6, 1 |
| | | | | culled day 9 - |
| | | | | weight loss >15% |
| | 0.5 mg/kg | No | 2 | All alive at day 14 |
| YTH54.12- | 5 mg/kg | No | 2 | All alive at day 14 |
| hIgG-AAA | | | | |
| (Unconjugated) | | | | |
| YTH54.12- | 5 mg/kg | No | 2 | All alive at day 14 |
| hIgG-AAA-PBD | | | | |
| | 3 mg/kg | Yes | 2 | 1 culled on day 9 |
| | | | | due to weight loss |
| | | | | >15% |
| | 0.5 mg/kg | No | 2 | All alive at day 14 |
| YTH24.5-rat | 5 mg/kg | No | 2 | All alive at day 14 |
| IgG2b | | | | |
| (unconjugated) | | | | |
| YTH24.5-rat | 5 mg/kg | No | 2 | All alive at day 14 |
| IgG2b-PBD | | | | |
| | 0.5 mg/kg | No | 2 | All alive at day 14 |
| YTH54.12-rat | 5 mg/kg | No | 2 | All alive at day 14 |
| IgG2b | | | | |
| (unconjugated) | | | | |
| YTH54.12-rat | 5 mg/kg | No | 2 | All alive at day 14 |
| IgG2b-PBD | | | | |
| | 0.5 mg/kg | No | 2 | All alive at day 14 |
| rat IgG2b isotype | 5 mg/kg | No | 2 | All alive at day 14 |
| control-PBD | | | | |
| | 0.5 mg/kg | No | 1 | Alive at day 14 |

It was noted that 4 of the 6 mice injected with YTH24.5-PBD ADC and 1 of the 6 mice treated with YTH54.12-PBD ADC in the human (hIgG1-AAA) format at ≥3 mg/kg had to be culled due to weight loss.

No culling was required for animals treated with ADCs in the rat (IgG2b) format.

At a dose of 3 mg/kg anti-CD45 (rat IgG2b) ADCs caused a reduction of bone marrow cellularity humanised NSG mice (see FIG. 10). The human CD45+ engraftment in the bone marrow of these mice was low (˜6%) as indicated by the PBS control group.

### Conclusions

These data showed that the rat IgG2b antibodies were better tolerated by the NSG mice. The rat IgG2b was the preferred format as it has been previously used in humans and had suitable PK parameters as conditioning agents. Higher doses of anti-human CD45 PBDs appeared to have an effect on both mouse and human cells in the bone marrow of humanised mice, therefore doses of 1 mg/kg or less were used for subsequent murine experiments. This toxicity may arise from bystander effect.

### Example 11: PK Studies

### Materials & Methods

Adult (non-humanized) NSG mice were injected with 1 mg/kg ADC. Three mice were used for each ADC. Samples were taken from mice at between 3 h and 168 h after injection. Serum samples were analyzed by ELISA using recombinant human CD45 (Bio-Techne). Goat anti-rat IgG (H+L) (cross absorbed) and sulfoTAG Streptavidin was used to detect the total amounts of the YTH antibodies. Anti-drug antibody was used to detect the level of conjugated antibody.

The drug-linker (DL) used in the assayed ADCs is “B1” as described herein.

### Results

See FIG. 11.

Similar PK profiles for both assayed anti-CD45 PBD ADCs, with AUC for YTH54.12-PBD ˜1.5 times greater than YTH24.5-PBD

### Conclusions

The YTH24.5-PBD and YTH54.12-PBD showed similar PK profiles and were below the lower limit of detection by ˜4 days after injection. These data support previous data (not shown) where the naked MAbs were not detectable at 7 days post injection.

### Example 12: Anti-Human CD45-PBDs Deplete Human CD45 Cells in Humanised NSG Mice

### Materials & Methods

Adult female NSG mice were sublethally irradiated with 2.5 Gy on the day before transplantation (day −57). Cryopreserved purified CD34+ cells from mobilized peripheral blood from a normal donor were thawed and 0.5×10⁶ cells in PBS were injected intravenously into the tail vein of mice (day −56). Human engraftment (human CD45+ cells) in the blood of mice was assessed at day −14 and day −1. Mice were assigned to treatment cohorts so that the median human CD45+ engraftment in the blood between the cohorts was as similar as possible. Mice were injected with 0.3 mg/kg or 1 mg/kg Mab or ADC intravenously via the tail vein. Blood samples were taken after 7 days and analysed by flow cytometry. The bone marrow, blood and spleen of mice were analysed at two weeks post-treatment.

The drug-linker (DL) used in the assayed ADCs is “B1” as described herein.

### Results (1)

FIG. 12

At Day −1 At 8 weeks, 40 of the 45 transplanted mice had blood human CD45+ engraftment levels between 1.4% and 28.4% (mean=6.10%). These 40 mice were divided into treatment groups with similar median engraftment levels for treatment with naked antibody or ADC on Day 0.

1 mg/kg anti-CD45-PBD dose was well tolerated in humanised mice, with no signs of toxicity observed (as indicated by no weight loss post-treatment with either naked antibody or PBD-ADC).

1 mg/kg YTH24.5 and YTH54.12 naked antibodies caused transient reduction in the levels of human CD45+ cells with subsequent recovery (see FIG. 12A).

1 mg/kg of either YTH PBD-ADC or 0.3 mg/kg YTH 24.5 PBD-ADC cause persistent loss of human CD45+ cells (see FIG. 12B).

### Conclusions/Summary (1)

| | | Mean % hCD45 levels in blood | | |
|---|---|---|---|---|
| | Dose | | 1 week | 2 weeks |
| | (mg/ | Pre- | post- | Post- |
| | kg) | treatment | treatment | treatment |
| PBS | | 5.88 | 7.89 | 4.89 |
| YTH24.5 | 1 | 5.47 | 0.82 | 3.92 |
| YTH24.5-PBD | 1 | 6.08 | 1.08 | 0.64 |
| YTH24.5 | 0.3 | 5.55 | 3.53 | 4.72 |
| YH24.5-PBD | 0.3 | 8.89 | 5.63 | 1.75 |
| YTH54.12 | 1 | 5.98 | 1.02 | 1.84 |
| YTH54.12-PBD | 1 | 6.14 | 0.75 | 0.38 |
| YTH54.12 | 0.3 | 5.52 | 3.19 | 3.81 |
| YTH54.12-PBD | 0.3 | 5.37 | 2.99 | 1.31 |

Anti-human CD45 naked antibodies lead to a transient decrease of hCD45+ve cells in blood. In contrast, anti-human PBDs lead to a dose-dependent and prolonged decrease in human CD45+ cells in blood.

### Results (2)

FIGS. 13-15

Anti-CD45 ADCs depleted human CD45+ cells in the blood, bone marrow and spleen of humanised NSG mice in a dose-dependent manner. 1 mg/kg anti-CD45 ADC effectively reduced the percentage human CD45+ cells to <0.6% vs 3.39% (PBS) in blood; to <0.34% vs 45.9% (PBS) in bone marrow; and to <0.12% vs 39.5% (PBS) in spleen (see FIG. 13).

Anti-CD45-PBDs depleted human CD45+ HSCs and progenitors in the bone marrow of humanized mice in a dose-dependent manner. 1 mg/kg anti-CD45 ADC effectively depleted to: <3000 total human CD34+ cells vs ˜2.8×10⁵ cells (PBS); and <27 human CD34+/hCD38-cells vs 1593 cells (PBS) (see FIG. 14).

Anti-CD45 ADCs depleted human CD45+ haematopoietic stem cells and multi-potent progenitors in the bone marrow of humanized mice in a dose-dependent manner. 1 mg/kg anti-CD45 ADC induce complete deletion of human HSC and MPPs in the bone marrow (see FIG. 15).

### Conclusions 2.

YTH24.5-PBD and YTH54.12-PBD specifically depleted immunophenotypically defined human HSCs and committed progenitors in humanized mice which supports their development as conditioning agents.

### Example 13: Anti-Human CD45-PBDs Enhances Engraftment of a Secondary Transplant in Humanized NSG Mice

### Materials & Methods

Adult female NSG mice were sublethally irradiated with 2.5 Gy on the day before transplantation (day −1). Cryopreserved purified CD34+ cells from mobilized peripheral blood from a normal donor were thawed and 0.5×10⁶ cells in PBS were injected intravenously into the tail vein of mice (day 0). Human engraftment (human CD45+ cells) in the blood of mice was assessed at week 8. Mice were assigned to treatment cohorts so that the median human CD45+ engraftment in the blood for each cohort was as similar as possible. Mice were injected with 1 mg/kg Mab or ADC or a combination of YTH MAbs (0.5 mg/kg each) or YTH ADCs (0.5 mg/kg each) intravenously via the tail vein. Mice were subsequently transplanted with lentivirally transduced green fluorescent protein (GFP) autologous CD34+ cells (Week 10). A separate cohort of sublethally irradiated (non-humanized) NSG were injected with the same batch of GFP transduced cells served as a positive control for GFP+ human cell engraftment. Nine weeks after GFP+ cell transplant, mice were culled and blood, spleen and bone marrow taken for analysis.

See FIG. 16 for approximate protocol timeline.

The drug-linker (DL) used in the assayed ADCs is “B1” as described herein.

### Results

FIG. 17.

The irradiated NSG mice which served as a positive control for the engraftment of GFP+ transduced cells indicate that the maximum level of GFP+ level that could be achieved by any antibody-based conditioning agents was ˜80% (see FIG. 17). In all the other treatment groups, GFP− human cells may be derived from the first transplant and also from the untransduced cells from the second transplant. GFP+ cells are derived solely from the second transplant. GFP+ levels in the bone marrow indicate efficacy of the Mab or ADC as conditioning agents to enable engraftment of GFP+ cells.

PBS, Isotype Mab, Isotype ADC, YTH24.5 Mab, YTH54.12 MAb or combination of the YTH MAbs, showed background engraftment levels of 30-40% GFP+ human CD45+ cells of all human CD45+ cells in the bone marrow (FIG. 17A).

Levels of GFP+ human CD45+ cell engraftment induced by the YTH24.5 ADC and YTH54.12 ADC or combination of the two all resulted in higher engraftment levels of GFP+ cells, up to 80%, compared to the isotype ADC treatment group in the bone marrow, showing anti-human CD45 ADCs were able to enhance the engraftment of GFP+ by depleting the cells from the first transplant. (FIG. 17A). These engraftment levels were also reflected in the myeloid (human CD33+) lineages and B cell (human CD19+) lineages (data not shown).

Analysis of the GFP+ human hematopoietic progenitor and HSC populations showed an increased level of GFP+CD34+ out of total human CD34+ cells (˜80%) in the bone marrow of mice treated with YTH24.5 ADC, YTH54.12 ADC and combination ADC groups compared to the isotype ADC control (˜40%) (FIG. 17B).

Analysis of total human HSCs, human MLPs and human MPPs showed that >80% were GFP+ in the YTH24.5 ADC and YTH54.12 ADC and combination ADC groups compared to the isotype ADC group (˜50 to 60%) (FIG. 17C-E).

### Conclusions

Treatment with anti-CD45 ADCs in humanized mice enabled the engraftment a second transplant of GFP+ autologous CD34+. The engraftment achieved was multilineage, with GFP+ cells detected in mature B cells and myeloid cells as well as in HSC and progenitors subpopulations. These data demonstrated that anti-CD45 ADCs specifically targeted and depleted human CD45+ cells in humanized mice and enabled enhanced engraftment of a second transplant of autologous GFP+ cells.

### Example 14: Anti-Human CD45-PBDs Show Anti-Leukaemic Activity in Murine Model of Leukemia

### Materials & Methods

Adult NSG mice were sublethally irradiated with 2 Gy on the day before injection of cells. 5×10⁶ fire fly luciferase expressing Jurkat cells in PBS were injected intravenously into the tail vein of mice. On the following day PBS, MAbs or ADC were injected intravenously into the tail vein at a dose of 1 mg/kg. Mice were regularly monitored for signs of morbidity and weight loss for the duration of the experiment. For imaging, mice were injected intraperitoneally with 3 mg D-luciferin in PBS. Mice were initially anesthetized with 4% isoflurane and then maintained at 2% isoflurane. Ten minutes after injection of D-luciferin, mice were imaged inside a IVIS Lumina III (PerkinElmer). A sub-saturating image was captured using the auto-exposure setting in the Living Image software.

The drug-linker (DL) used in the assayed ADCs is “B1” as described herein.

### Results

See FIG. 18.

Rapid leukaemia development was observed in mice injected with Jurkat cells.

Administration of anti-human CD45 naked antibodies delayed development of leukaemia but the delay was transient.

Administration of anti-CD45-ADCs largely delayed the onset of leukaemia over the course of the IVIS study and prolonged survival.

PBS, isotype and isotype-ADC controls have 50 fold higher signal than all other treatment groups at day 16.

Anti-CD45 ADCs signals were markedly lower than anti-CD45 naked antibody groups at day 16.

YTH24.5 naked antibody group has 100-old higher signal than YTH24.5-ADC group (where 5 of 5 mice remained leukaemia free) at day 16.

YTH54.12 naked antibody group had 10-fold higher signal than YTH54.12-ADC group (where 4 of 5 mice remained leukaemia free) at day 16.

At end of day 70, only the YTH24.5 ADC and YTH54.12 ADC groups had surviving mice.

### Conclusions

YTH24.5 and YTH54.12 MAbs delayed the onset of disease in a xenogeneic model of leukemia and conferred some survival advantage over PBS, Isotype Mab and Isotype ADC groups. Anti-human CD45 ADCs further delayed disease progression and further prolonged the survival of leukemic mice compared to naked anti-human CD45 Mab treatment groups.

### Example 15: In Vitro Comparison of Anti-CD45 Antibodies Conjugated to Different PBD Payloads

### Materials & Methods

OCIM1, Jurkat and Nalm6 cells were seeded at a final concentration of 1.275×10⁴/mL and for 5 days in presence of rat IgG2b Mab or ADC or medium (in triplicate) in wells of flat bottom 96-well plates. 293T cells were seeded at 9.3×10³ cells per 100 uL per well in flat bottom 96-well plates and allowed to adhere the plate for up to 24 hours. 293T cells were subsequently cultured with rat IgG2b Mabs or ADC or medium (in triplicate wells) as described above. PrestoBlue® Cell viability Reagent (Thermo Fisher Scientific) was added to each and incubated for up to 1.5 hours. Fluorescence intensity from each well was detected using a FLUOstar OPTIMA microplate reader (BMG Labtech). Data was plotted using Prism 8.0 and sigmoid dose-response non-linear regression was used to determine EC₅₀ values. Data points are mean±s.d. for representative experiments.

### Results

See FIG. 20 and summary table.

Isotype and anti-CD45-specific antibodies were conjugated to different payloads of different potencies and ability to induce bystander effect. These ADCs were compared for their ability to kill CD45+ and CD45-cell lines in cell viability assays in vitro. Anti-CD45 antibodies conjugated to B4 were 2- to 3-fold more potent than those conjugated to B1 on CD45-positive cells, with Isotype-B4 showing less non-specific toxicity compared to Isotype-B1 (FIG. 20A, FIG. 20B and summary table above). The anti-CD45-B2 and anti-CD45-B7 ADCs also specifically killed antigen-positive cells (FIG. 20E and FIG. 20F and summary table), but were less potent than the B1 and B4 ADCs. With antigen-negative Nalm6 and HEK293T cell lines (FIG. 20C, FIG. 20D, FIG. 208G and FIG. 20H and summary table), there was non-specific cell killing by both anti-CD45 ADCs and Isotype ADCs at the highest concentrations tested.

### Summary Table: Comparison of EC₅₀ Values for Different Anti-CD45 ADCs on Cell Lines.

EC₅₀ values were determined from 3 to 4 independent experiments.

| | Jurkat | | OCIM1 | | Nalm6 | | 293T | |
|---|---|---|---|---|---|---|---|---|
| | (CD45+) | | (CD45+) | | (CD45−) | | (CD45−) | |
| | Average | | Average | | Average | | Average | |
| | EC50 | | EC50 | | EC50 | | EC50 | |
| ADC | (pM) | n | (pM) | n | (pM) | n | (pM) | n |
| YTH24.5-B1 | 0.213 | 4 | 0.030 | 4 | 9.34 | 3 | >6,000 | 3 |
| YTH54.12-B1 | 0.127 | 4 | 0.039 | 4 | 9.64 | 3 | >6,000 | 3 |
| Isotype-B1 | 309.6 | 4 | 214.4 | 4 | 18.41 | 3 | >6,000 | 3 |
| YTH24.5-B4 | 0.052 | 4 | 0.096 | 4 | 161.89 | 3 | >6,000 | 3 |
| YTH54.12-B4 | 0.091 | 4 | 0.220 | 4 | 714.65 | 3 | >6,000 | 3 |
| Isotype-B4 | 3,399.8 | 4 | 2,288.3 | 4 | 697.58 | 3 | >6,000 | 3 |
| YTH24.5-B2 | 2.123 | 4 | 0.025 | 4 | 30.13 | 3 | >6,000 | 3 |
| YTH54.12-B2 | 2.429 | 4 | 0.580 | 4 | 27.85 | 3 | >6,000 | 3 |
| Isotype-B2 | 1,653.3 | 4 | 1,511.04 | 4 | 55.05 | 3 | >6,000 | 3 |
| YTH24.5-B7 | 8.40 | 4 | 2.18 | 4 | 904.6 | 3 | >6,000 | 3 |
| YTH54.12-B7 | 4.79 | 4 | 1.07 | 4 | 1,555.99 | 3 | >6,000 | 3 |
| Isotype-B7 | 60,255.3 | 4 | 5,806.02 | 4 | 1,753.10 | 3 | >6,000 | 3 |

### Conclusions

All anti-CD45 ADCs were specific for CD45+ cell lines. However, anti-CD45-specific antibodies conjugated to B1 and B4 were more potent than the B2 and B7 payloads on CD45+ cell lines. The Isotype-B4 was less toxic than Isotype-B1 on CD45+ cells, most likely due to lack of bystander effect in the former.

### Example 16: Comparison of Different Payloads in Clonogenic Assays

### Materials and Methods

Cryopreserved purified CD34+ cells from mobilized peripheral blood of healthy donors were thawed, washed and resuspended at a final cell density of 0.5×10⁵/mL in RPMI-1640 medium plus 10% fetal bovine serum in presence of rat IgG2b Isotype ADC, or rat IgG2b anti-CD45 ADC, or media alone or for 2 hours. 1000 cells were transferred to 2 ml of StemMACS HSC-CFU complete with Epo (Miltenyi Biotech) and mixed well. 0.5 ml was plated, in triplicate, into wells of a 24-well plate. Colonies were enumerated after 12-14 days. Data was plotted using Prism 8.0 and sigmoid dose-response non-linear regression was performed. Pooled data from three donors was expressed relative to the untreated cells±s.d. EC₅₀ values were determined as the concentration at which gave 50% response.

### Results

See FIG. 21 and summary table.

Anti-CD45-specific and Isotype antibodies were conjugated to different PBD payloads and their ability to inhibit colony formation in clonogenic assays was determined. Clonogenic assays showed that the anti-CD45 antibodies conjugated to either B1 and B4 had similar potencies on inhibition of colony formation by primary human CD34+ cells, with no significant difference between the two clones (FIG. 21A and FIG. 21B and summary table above). Isotype-B1 resulted in inhibition of colony formation at the highest concentrations tested, whereas Isotype-B4 showed no inhibition in colony formation at the concentrations tested. Anti-CD45-B2 and anti-CD45-B7 ADCs specifically inhibited colony formation, but were less potent than the B1 and B4 ADCs (FIG. 21C and FIG. 21D and summary table). Isotype-B2 and Isotype-B7 resulted in non-specific inhibition of colony formation at the highest concentration tested.

### Summary Table: Comparison of Anti-CD45 Antibodies Conjugated to B1, B4, B2 and B7 in Clonogenic Assays.

| ADC | Average EC₅₀ (pM) | n |
|---|---|---|
| YTH24.5-B1 | 2.64 | 3 |
| YTH54.12-B1 | 1.52 | 3 |
| Isotype-B1 | 3,593.52 | 3 |
| YTH24.5-B4 | 4.92 | 3 |
| YTH54.12-B4 | 4.47 | 3 |
| Isotype-B4 | >100,000 | 3 |
| YTH24.5-B2 | 32.71 | 3 |
| YTH54.12-B2 | 23.86 | 3 |
| Isotype-B2 | 21,215.98 | 3 |
| YTH24.5-B7 | 35.53 | 3 |
| YTH54.12-B7 | 45.10 | 3 |
| Isotype-B7 | 72,118.56 | 3 |

### Conclusions

Anti-CD45-B1 and anti-CD45-B4 are equally effective in clonogenic assays in inhibiting colony formation of primary human CD34+ cells (which co-express CD45) from mobilized peripheral blood. However, Isotype-B4 demonstrated less non-specific toxicity and so is preferable overall. Anti-CD45-B2 and anti-CD45-B7 were less potent than anti-CD45-B1 and anti-CD45-B4, with Isotype-B2 and -B7 inducing non-specific killing at high concentrations.

### Example 17. Comparison of Different Payloads in AML Model in NSG Mice

### Materials and Methods

NSG mice were transplanted with 7.5×10⁶ luciferase expressing fLuc+ OCIM1 cells i.v. On the following day, unconjugated rat IgG2b antibodies or ADCs were injected i.v. via a tail vein at a dose of 1 mg/kg or 5 mg/kg. Mice were regularly monitored for signs of morbidity for the duration of the experiment. For imaging, mice were injected intraperitoneally with 200 μl of 15 mg/mL D-luciferin (Regis Technologies) in PBS. Mice were subsequently anesthetized with 4% isoflurane and maintained at 2% isoflurane. Ten minutes after injection of D-luciferin, mice were placed with anterior side up inside a IVIS Lumina III (PerkinElmer). A sub-saturating image was captured using the auto-exposure setting in the Living Image software (v4.5). Quantification and preparation of images was also carried out using the same software.

### Results

See FIG. 22.

The relative anti-leukemic activity of anti-CD45-specific and isotype antibody conjugated to different PBD payloads were assessed a NSG AML model. All mice in control groups (unconjugated antibody or Isotype ADC) showed increasing tumor signal over time (FIG. 22A to FIG. 22D) and were all culled by day 32 (FIG. 22E). YTH54.12-B1 showed increasing tumor signal from day 18 onwards (due to tumor development in 1 out of 5 mice) (FIG. 22A). Low/absent tumor signal was detected in the YTH24.5-B1, YTH24.5-B4 and YTH24.5-B2 treated mice (FIG. 22A, FIG. 22B and FIG. 22C). Increasing tumor signal in three mice treated with YTH24.5-B7 was detected from day 11 (FIG. 22D).

### Conclusions

YTH24.5 conjugated to high potency payloads, B1 and B4, and to the lower potency B2 and B7 payloads were able to prevent tumor development in a NSG model of AML. YTH54.12-B1 was marginally less effective than YTH24.5-B1, as YTH24.5-B1 treatment resulted in 1 out of 5 mice developing a low tumor signal. YTH24.5-B7 was the least potent ADC, with 3/5 mice showing high levels of tumor signal by the end of the experiment. Nevertheless, all the anti-CD45 ADCs tested prolonged the survival of mice compared to the control groups.

### Example 18. Comparison of Anti-CD45 ADCs with Different Payloads in Established Model of AML in NSG Mice

### Materials and Methods

NSG mice were transplanted with 7.5×10⁶ fLuc+ OCIM1 cells i.v. At 10 days post-injection of AML cells, unconjugated antibody or ADCs were injected i.v. via a tail vein at a dose of 1 mg/kg or 5 mg/kg. Mice were regularly monitored for signs of morbidity for the duration of the experiment. For imaging, mice were injected intraperitoneally with 200 μl of 15 mg/mL D-luciferin (Regis Technologies) in PBS. Mice were subsequently anesthetized with 4% isoflurane and maintained at 2% isoflurane. Ten minutes after injection of D-luciferin, mice were placed with anterior side up inside a IVIS Lumina III (PerkinElmer). A sub-saturating image was captured using the auto-exposure setting in the Living Image software (v4.5). Quantification and preparation of images was also carried out using the same software.

### Results

See FIG. 23.

The relative anti-leukemic activity of anti-CD45-specific and isotype antibody conjugated to different PBD payloads were assessed on established tumors in a NSG AML model. Mice injected with fLuc+ OCIM1 cells showed approximately a 5-fold increase in bioluminescence signal before ADC treatment was given (FIG. 23A). Unconjugated antibody control groups and Isotype-B1, Isotype-B4 and Isotype-B2 treated mice all developed increasing tumor signal from the onset of the experiment (FIG. 23A) and all mice in these were culled by day 39 due to morbidity (FIG. 23B). The anti-CD45-specific ADCs all induced tumor regression within 7 days of treatment (FIG. 23A), with YTH24.5-B4 inducing a marginally longer period of low tumor signal than YTH24.5-B1. However, YTH24.5-B2 induced the shortest period of tumor regression and tumor signal increased within 10 days. At 60 days, all mice were tumor-bearing, but 1/5 mice in B2 group was alive, whilst 4/5 mice in the YTH24.5-B1 and YTH54.12-B4 were alive.

### Conclusions

All anti-CD45-ADCs tested in mice with established AML resulted in regression of tumor signal and prolonged survival compared to control mice. However, YTH24.5-B4 was the most effective at inducing persistent tumour regression and prolonged survival, followed by YTH24.5-B1 and YTH24.5-B2 in that order.

### Example 19. Assessment of YTH24.5-B1 as a Conditioning Agent in an Allogeneic Stem Cell Transplant Model in NSG Mice

### Materials and Methods

Adult NSG mice (Charles River Laboratories) were sublethally irradiated with 2.5 Gy in a ¹³⁷Cs irradiator. On the following day, mice were injected with 0.5×10⁶ freshly thawed cryopreserved human CD34+ cells from a health donor were injected via the tail vein in 150 μL PBS using a 29 gauge MicroFine Plus insulin syringe (BD). Engraftment of human cells in the blood was assessed periodically from 6 weeks after transplantation. At 10 weeks post-transplant, humanized NSG mice were assigned into cohorts with similar median human CD45+ cell engraftment in the blood, as determined by flow cytometry. Mice were injected with PBS or 1 mg/kg YTH24.5-B1. Sixteen 16 days after treatment, mice were transplanted with 0.5×10⁶ GFP+CD34+ cells from another healthy donor. Irradiated NSG which had no non-humanized mice were also transplanted with the GFP+CD34+ from the allogeneic healthy donor as a control for engraftment of the GFP+CD34+ cells. 8 weeks after transplantation of GFP+ cells, mice were culled and blood, spleen and bone marrow taken for analysis by flow cytometry.

### Results

See FIG. 24.

Human CD45+ cells engraftment levels in the bone marrow of mice conditioned with YTH24.5-B1 group was statistically significantly lower compared to the PBS treated group (mean % hCD45=9.08% and 37.5%, respectively) (FIG. 24A). However, the percentage GFP+hCD45+ cells of all human CD45+ cells in bone marrow were statistically significantly higher with the YTH24.5-B1 (mean=29.0%) treatment compared to the PBS group (mean=2.1%) (FIG. 24B). Irradiation control mice had a mean GFP+hCD45+ level of 41.3%. YTH24.5-B1 also resulted in statistically significantly higher levels of GFP+hCD34+ cells (mean=56.6%) compared to PBS treatment (mean=3.7%) within the human CD34+ compartment in the bone marrow (FIG. 24C).

### Conclusions

These data show that YTH24.5-B1 treatment but not PBS treatment was able to condition humanized mice to enable engraftment of cells from an allogeneic human donor.

### Example 20. Comparison of Anti-CD45 ADCs with Different Payloads in an Allogeneic Stem Cell Transplant Model in NSG Mice

### Materials and Methods

Experimental schedule is as previously described in example 16 but with the following modifications. Humanized NSG mice at 10 weeks post-transplant were injected with PBS or 1 mg/kg YTH24.5-B1, YTH54.12-B1, Isotype-B1, YTH24.5-B4 and Isotype-3376. Isotype-B2 and YTH24.5-B2 was given at a dose of 5 mg/kg. Transplantation of 0.5×10⁶ GFP+CD34+ from an allogeneic human donor and analysis at 8 weeks post-transplant of GFP+ cells was carried out as previously described.

### Results

See FIG. 25.

Isotype and anti-CD45-specific antibodies with different PBD payloads were compared for their ability to condition humanized mice and facilitate engraftment of GFP+CD34+ cells from a human allogeneic donor. The mean human CD45+ cell engraftment level in the bone marrow of mice treated with PBS, followed by transplantation of GFP+CD34+ allogeneic cells was 14.12% (FIG. 25A). There was no statistically significant difference in human CD45+ engraftment between Isotype-B1 (mean % hCD45+=35.34%) compared to YTH24.5-B1 (mean % hCD45+=16.45%) and YTH54.12-B1 (mean % hCD45+=16.34%, nor Isotype-B4 (mean % hCD45+=15.22%) compared YTH24.5-B4 (mean % hCD45+=7.34%) (FIG. 25A). However, YTH24.5-B2 resulted in a statistically significant decrease in human CD45+ engraftment levels (mean % hCD45+=1.089%) compared to Isotype-B2 (mean % hCD45+=7.06%). Highest levels of human CD45+ cell engraftment was observed in the irradiation control cohort, i.e. mice which received GFP+CD34+ cells after 2.5 Gy irradiation (with no other prior conditioning or humanization).

The mean percentage GFP+hCD45+ cells of all human CD45+ cells in bone marrow after PBS treatment was 10.22% (FIG. 25B). The mean percentage GFP+hCD45+ engraftment in the cohorts treated with Isotype-B1, YTH24.5-B1 and YTH54.12-61, which ranged between 33.64% and 55.56%, were higher compared to all the other ADC or PBS groups (FIG. 25B). However, there were no statistically significant differences between Isotype-B1, YTH24.5-B1 and YTH54.12-B1 in percentage GFP+ human CD45+ cells. There were also no statistically significant differences between Isotype-B4 compared to YTH24.5-B4, nor Isotype-B2 compared to YTH24.5-B2 in the percentage of GFP+ human CD45+ cells.

When the levels of GFP+ human CD34+ cells (FIG. 23C) were assessed, there was a statistically significantly increase in GFP+ human CD34+ cells in the bone marrow of mice which received YTH24.5-B4 (mean=57.95%) and YTH24.5-B2 (72.14%) compared to their Isotype ADC controls (means=24.42% and 14.92%, respectively). Although the percentage GFP+ human CD34+ was higher in the Isotype-B1, YTH24.5-B1 and YTH54.12-B1 cohorts (50.09%, 59.13% and 69.40%, respectively), there was no difference between the non-specific and anti-CD45-specific B1 ADCs in this experiment.

### Conclusions

These data show that anti-CD45-PBD ADCs, with payloads of different potencies and bystander activity, result in different levels of depletion of human cells in humanized mice. Unexpectedly, the Isotype-B1 and anti-CD45-B1 ADCs appeared to show equivalent activity (high total human CD45+ engraftment levels), with no apparent difference in specific and non-specific ADC activity. This was not observed in the autologous stem cell transplant model. In this experiment, YTH24.5-B4 compared to Isotype-B4 resulted in significant levels of human CD45+ cell engraftment and GFP+ human CD45+ engraftment. Although, YTH24.5-B2 induced significant levels of GFP+ human CD34+ cell engraftment compared to Isoytpe-B2, overall human CD45+ engraftment was low. Further optimization of ADCs dose and other parameters, e.g. time post-ADC treatment before transplant of GFP+ human CD34+ cells, may be necessary for optimal conditioning by anti-CD45 ADCs with different payloads.

### SEQUENCE LISTING PART OF THE DESCRIPTION

[YTH24.5 VH, CDR underline]
SEQ ID NO. 1
QVNLLQSGAALVKPGASVKLSCKASSYTFTDYYIHWVKQSHGKTLEWIGYINPKSGFTNYN
EKFRRKATLTVDKSTNTAYMDISRLTSEDSATYYCTRRTGVIPMDAWGQGASVTVSS
[YTH24.5 VL, CDR underline]
SEQ ID NO. 2
DVVMTQTPVSLSVSLGGQVSISCRSSQSFVSSDGNTYLNWYLQKPGQSPQLLIYKVSNRLS
GVPDRFSGSGSGTDFTLKISRVEHDDLGVYYCGQASKIPLTFGSGTKLEIK
[YTH24.5 VH, CDR1]
SEQ ID NO. 3
DYYIH
[YTH24.5 VH, CDR2]
SEQ ID NO. 4
YINPKSGFTNYNEKFRR
[YTH24.5 VH, CDR3]
SEQ ID NO. 5
RTGVIPMDA
[YTH24.5 VL, CDR1]
SEQ ID NO. 6
RSSQSFVSSDGNTYLN
[YTH24.5 VL, CDR2]
SEQ ID NO. 7
KVSNRLS
[YTH24.5 VL, CDR3]
SEQ ID NO. 8
GQASKIPLT
[YTH24.5 Heavy Chain (Rat IgG2b, kappa)]
SEQ ID NO. 9
QVNLLQSGAALVKPGASVKLSCKASSYTFTDYYIHWVKQSHGKTLEWIGYINPKSGFTNYN
EKFRRKATLTVDKSTNTAYMDISRLTSEDSATYYCTRRTGVIPMDAWGQGASVTVSSAQTT
APSVYPLAPGCGDTTSSTVTLGCLVKGYFPEPVTVTWNSGALSSDVHTFPAVLQSGLYTLT
SSVTSSTWPSQTVTCNVAHPASSTKVDKKVERRNGGIGHKCPTCPTCHKCPVPELLGGPS
VFIFPPKPKDILLISQNAKVTCVVVDVSEEEPDVQFSWFVNNVEVHTAQTQPREEQYNSTFR
VVSALPIQHQDWMSGKEFKCKVNNKALPSPIEKTISKPKGLVRKPQVYVMGPPTEQLTEQT
VSLTCLTSGFLPNDIGVEWTSNGHIEKNYKNTEPVMDSDGSFFMYSKLNVERSRWDSRAP
FVCSVVHEGLHNHHVEKSISRPPGK
[YTH24.5 Light Chain (Rat IgG2b, kappa)]
SEQ ID NO. 10
DVVMTQTPVSLSVSLGGQVSISCRSSQSFVSSDGNTYLNWYLQKPGQSPQLLIYKVSNRLS
GVPDRFSGSGSGTDFTLKISRVEHDDLGVYYCGQASKIPLTFGSGTKLEIKRADAAPTVSIF
PPSMEQLTSGGATVVCFVNNFYPRDISVKWKIDGSEQRDGVLDSVTDQDSKDSTYSMSST
LSLTKVEYERHNLYTCEVVHKTSSSPVVKSFNRNEC
[YTH54.12 VH, CDR underline]
SEQ ID NO. 11
EVQLVESGGGLVQPGGSMKLSCAASGFTFSDYYMAWVRQAPKKGLEWVASMSFAGSST
YYGDSVKGRFTISRDNAKTTLYLQMNSLRSEDTATYYCARMYTTDYYLYWYFDFWGPGTM
VTVSS
[YTH54.12 VL, CDR underline]
SEQ ID NO. 12
DVQMTQSPSYLAASPGESVSISCKASKSISNYLAWYQQKPGEANKILIYSGSTLQSGTPSRF
SGSGSGTDFSLTIRNLEPEDFAVYYCQQYDEKPLTFGSGTKLEIK
[YTH54.12 VH, CDR1]
SEQ ID NO. 13
DYYMA
[YTH54.12 VH, CDR2]
SEQ ID NO. 14
SMSFAGSSTYYGDSVKG
[YTH54.12 VH, CDR3]
SEQ ID NO. 15
MYTTDYYLYWYFDF
[YTH54.12 VL, CDR1]
SEQ ID NO. 16
KASKSISNYLA
[YTH54.12 VL, CDR2]
SEQ ID NO. 17
SGSTLQS
[YTH54.12 VL, CDR3]
SEQ ID NO. 18
QQYDEKPLT
[YTH54.12 Heavy Chain (Rat IgG2b, kappa)]
SEQ ID NO. 19
EVQLVESGGGLVQPGGSMKLSCAASGFTFSDYYMAWVRQAPKKGLEWVASMSFAGSST
YYGDSVKGRFTISRDNAKTTLYLQMNSLRSEDTATYYCARMYTTDYYLYWYFDFWGPGTM
VTVSSAQTTAPSVYPLAPGCGDTTSSTVTLGCLVKGYFPEPVTVTWNSGALSSDVHTFPAV
LQSGLYTLTSSVTSSTWPSQTVTCNVAHPASSTKVDKKVERRNGGIGHKCPTCPTCHKCP
VPELLGGPSVFIFPPKPKDILLISQNAKVTCVVVDVSEEEPDVQFSWFVNNVEVHTAQTQPR
EEQYNSTFRVVSALPIQHQDWMSGKEFKCKVNNKALPSPIEKTISKPKGLVRKPQVYVMGP
PTEQLTEQTVSLTCLTSGFLPNDIGVEWTSNGHIEKNYKNTEPVMDSDGSFFMYSKLNVER
SRWDSRAPFVCSVVHEGLHNHHVEKSISRPPGK
[YTH54.12 Light Chain (Rat IgG2b, kappa)]
SEQ ID NO. 20
DVQMTQSPSYLAASPGESVSISCKASKSISNYLAWYQQKPGEANKILIYSGSTLQSGTPSRF
SGSGSGTDFSLTIRNLEPEDFAVYYCQQYDEKPLTFGSGTKLEIKRADAAPTVSIFPPSMEQ
LTSGGATVVCFVNNFYPRDISVKWKIDGSEQRDGVLDSVTDQDSKDSTYSMSSTLSLTKVE
YERHNLYTCEVVHKTSSSPVVKSFNRNEC
[human CD45]
SEQ ID NO. 21
MTMYLWLKLLAFGFAFLDTEVFVTGQSPTPSPTGLTTAKMPSVPLSSDPLPTHTTAFSPAST
FERENDFSETTTSLSPDNTSTQVSPDSLDNASAFNTTGVSSVQTPHLPTHADSQTPSAGTD
TQTFSGSAANAKLNPTPGSNAISDVPGERSTASTFPTDPVSPLTTTLSLAHHSSAALPARTS
NTTITANTSDAYLNASETTTLSPSGSAVISTTTIATTPSKPTCDEKYANITVDYLYNKETKLFTA
KLNVNENVECGNNTCTNNEVHNLTECKNASVSISHNSCTAPDKTLILDVPPGVEKFQLHDC
TQVEKADTTICLKWKNIETFTCDTQNITYRFQCGNMIFDNKEIKLENLEPEHEYKCDSEILYN
NHKFTNASKIIKTDFGSPGEPQIIFCRSEAAHQGVITWNPPQRSFHNFTLCYIKETEKDCLNL
DKNLIKYDLQNLKPYTKYVLSLHAYIIAKVQRNGSAAMCHFTTKSAPPSQVWNMTVSMTSD
NSMHVKCRPPRDRNGPHERYHLEVEAGNTLVRNESHKNCDFRVKDLQYSTDYTFKAYFH
NGDYPGEPFILHHSTSYNSKALIAFLAFLIIVTSIALLVVLYKIYDLHKKRSCNLDEQQELVERD
DEKQLMNVEPIHADILLETYKRKIADEGRLFLAEFQSIPRVFSKFPIKEARKPFNQNKNRYVDI
LPYDYNRVELSEINGDAGSNYINASYIDGFKEPRKYIAAQGPRDETVDDFWRMIWEQKATVI
VMVTRCEEGNRNKCAEYWPSMEEGTRAFGDVVVKINQHKRCPDYIIQKLNIVNKKEKATGR
EVTHIQFTSWPDHGVPEDPHLLLKLRRRVNAFSNFFSGPIVVHCSAGVGRTGTYIGIDAMLE
GLEAENKVDVYGYVVKLRRQRCLMVQVEAQYILIHQALVEYNQFGETEVNLSELHPYLHNM
KKRDPPSEPSPLEAEFQRLPSYRSWRTQHIGNQEENKSKNRNSNVIPYDYNRVPLKHELE
MSKESEHDSDESSDDDSDSEEPSKYINASFIMSYWKPEVMIAAQGPLKETIGDFWQMIFQR
KVKVIVMLTELKHGDQEICAQYWGEGKQTYGDIEVDLKDTDKSSTYTLRVFELRHSKRKDS
RTVYQYQYTNWSVEQLPAEPKELISMIQVVKQKLPQKNSSEGNKHHKSTPLLIHCRDGSQQ
TGIFCALLNLLESAETEEVVDIFQVVKALRKARPGMVSTFEQYQFLYDVIASTYPAQNGQVK
KNNHQEDKIEFDNEVDKVKQDANCVNPLGAPEKLPEAKEQAEGSEPTSGTEGPEHSVNGP
ASPALNQGS

1. A conjugate of formula (I):
Ab-(DL)ₚ (I)
wherein:
Ab is an antibody that binds to CD45; and DL is:

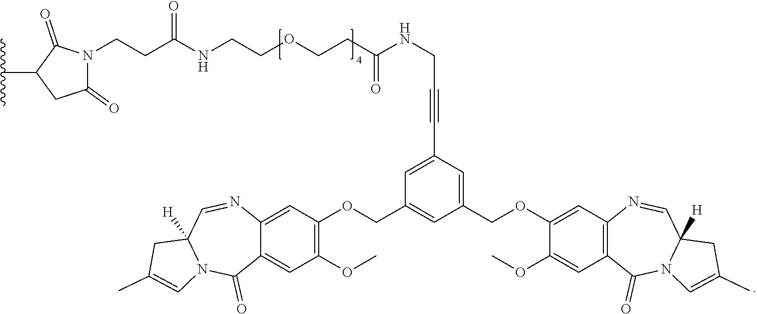

2. The conjugate according to any claim 1, wherein p is 1 to 4.

3. The conjugate according to claim 1, wherein the antibody comprises a VH domain comprising a VH CDR3 with the amino acid sequence of SEQ ID NO. 5., a VH CDR2 with the amino acid sequence of SEQ ID NO. 4, and a VH CDR1 with the amino acid sequence of SEQ ID NO. 3.

4. The conjugate according to claim 1, wherein the antibody comprises a VH domain having the sequence of SEQ ID NO. 1.

5. The conjugate according to claim 3, wherein the antibody comprises a VL domain comprising a VL CDR3 with the amino acid sequence of SEQ ID NO. 8., a VL CDR2 with the amino acid sequence of SEQ ID NO. 7, and a VL CDR1 with the amino acid sequence of SEQ ID NO. 6.

6. The conjugate according to claim 4, wherein the antibody comprises a VL domain having the sequence of SEQ ID NO. 2.

7. The conjugate according to claim 1, wherein the antibody comprises a VH domain comprising a VH CDR3 with the amino acid sequence of SEQ ID NO. 15., a VH CDR2 with the amino acid sequence of SEQ ID NO. 14, and a VH CDR1 with the amino acid sequence of SEQ ID NO. 13.

8. The conjugate according to claim 1, wherein the antibody comprises a VH domain having the sequence of SEQ ID NO. 11.

9. The conjugate according to claim 7, wherein the antibody comprises a VL domain comprising a VL CDR3 with the amino acid sequence of SEQ ID NO. 18., a VL CDR2 with the amino acid sequence of SEQ ID NO. 17, and a VL CDR1 with the amino acid sequence of SEQ ID NO. 16.

10. The conjugate according to claim 8, wherein the antibody comprises a VL domain having the sequence of SEQ ID NO. 12.

11. The conjugate according to claim 1, wherein the antibody comprises a first antigen binding domain and a second antigen binding domain, wherein:
the first antigen binding domain comprises a VH domain comprising a VH CDR3 with the amino acid sequence of SEQ ID NO. 5., a VH CDR2 with the amino acid sequence of SEQ ID NO. 4, and a VH CDR1 with the amino acid sequence of SEQ ID NO. 3 and/or a VL domain comprising a VL CDR3 with the amino acid sequence of SEQ ID NO. 8., a VL CDR2 with the amino acid sequence of SEQ ID NO. 7, and a VL CDR1 with the amino acid sequence of SEQ ID NO. 6; and
the second antigen binding domain comprises a VH domain comprising a VH CDR3 with the amino acid sequence of SEQ ID NO. 15., a VH CDR2 with the amino acid sequence of SEQ ID NO. 14, and a VH CDR1 with the amino acid sequence of SEQ ID NO. 13 and/or a VL domain comprising a VL CDR3 with the amino acid sequence of SEQ ID NO. 18., a VL CDR2 with the amino acid sequence of SEQ ID NO. 17, and a VL CDR1 with the amino acid sequence of SEQ ID NO. 16.

12. The conjugate according to claim 1, wherein the antibody in an intact antibody.

13. The conjugate according to claim 1, wherein said antibody is non-human.

14. The conjugate according to claim 1, wherein the antibody is a rat antibody such as a rat IgG2b.

15. A method of treating cancer, the method comprising administering an effective amount of the conjugate according claim 1 to a subject in need thereof.

16. A pharmaceutical composition comprising a first conjugate of formula (I), a second conjugate of formula (I), and optionally a pharmaceutically acceptable diluent, carrier or excipient,
$$( I ) \text{:}$$
$$Ab - {( DL )}_{p} \qquad ( I )$$
wherein:
Ab is an antibody that binds to CD45; and DL is: 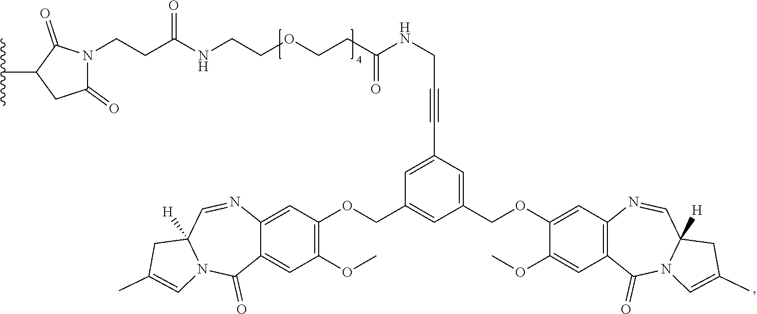
and wherein:
(a) the antibody of the first conjugate comprises a VH domain comprising a VH CDR3 with the amino acid sequence of SEQ ID NO. 5., a VH CDR2 with the amino acid sequence of SEQ ID NO. 4, and a VH CDR1 with the amino acid sequence of SEQ ID NO. 3 and a VL domain comprising a VL CDR3 with the amino acid sequence of SEQ ID NO. 8., a VL CDR2 with the amino acid sequence of SEQ ID NO. 7, and a VL CDR1 with the amino acid sequence of SEQ ID NO. 6; and
(b) the antibody of the second conjugate comprises a VH domain comprising a VH CDR3 with the amino acid sequence of SEQ ID NO. 15., a VH CDR2 with the amino acid sequence of SEQ ID NO. 14, and a VH CDR1 with the amino acid sequence of SEQ ID NO. 13 and a VL domain comprising a VL CDR3 with the amino acid sequence of SEQ ID NO. 18., a VL CDR2 with the amino acid sequence of SEQ ID NO. 17, and a VL CDR1 with the amino acid sequence of SEQ ID NO. 16.

17. A method of treating haematological cancer, the method comprising administering the conjugate according to claim 1 to a subject in need thereof.

18. The method of claim 17, wherein the haematological cancer is selected from the group consisting of: acute myeloid leukaemia, acute lymphoblastic leukaemia, chronic lymphocytic leukaemia, chronic myeloid leukaemia, myelodysplasia, multiple myeloma, non-Hodgkin's lymphoma and Hodgkin's disease.

19. A method of preparing a subject for transplantation of haematopoietic stem cells, the method comprising administering the conjugate according to claim 1 to the subject.

20. The method of claim 19 wherein the preparing for transplantation of haematopoietic stem cells comprises conditioning the subject for engraftment of haematopoietic stem cells.

21. The method of claim 19, wherein the haematopoietic stem cells are allogeneic.

22. The method of claim 21, wherein said transplantation of haematopoietic stem cells is for treating a malignant disease or disorder, optionally selected from the group consisting of: acute myeloid leukaemia, acute lymphoblastic leukaemia, chronic myeloid leukaemia, chronic lymphocytic leukaemia, myelodysplasia, myeloproliferative diseases, non-Hodgkin's lymphoma and Hodgkin's disease.

23. The method of claim 21, wherein said transplantation of haematopoietic stem cells is for treating a non-malignant disease or disorder, optionally selected from the group consisting of: severe aplastic anaemia, a bone marrow failure disorder, a primary immunodeficiency, a haemoglobinopathy, primary haemophagocytic lymphohistiocytosis and a genetic metabolic disease.

24. The method of claim 21, wherein said transplantation of haematopoietic stem cells is for treating:
(i) a bone marrow failure disorder selected from Fanconi anaemia, dyskeratosis congenital, Schwachmann-Diamond Syndrome;
(ii) a primary immunodeficiency selected from SCID, chronic granulomatous disease, Wiskott-Aldrich syndrome, CD40 ligand deficiency, XLP, MHC Class II deficiency;
(i) a haemoglobinopathy selected from sickle cell disease, β-thalassaemia major; or
(ii) a genetic metabolic disease selected from Hurler syndrome, X-linked adrenoleukodystrophy and osteopetrosis.

25. The method of claim 19, wherein the haematopoietic stem cells are autologous.

26. The method of claim 25, wherein said transplantation of haematopoietic stem cells is for treating a malignant disease or disorder, optionally selected from the group consisting of: multiple myeloma, non-Hodgkin's lymphoma, Hodgkin's disease.

27. The claim of method 25, wherein said transplantation of haematopoietic stem cells is for treating an autoimmune disease or disorder, optionally selected from the group consisting of: multiple sclerosis, systemic sclerosis and systemic lupus erythematosus.

28. The method of claim 19, wherein the haematopoietic stem cells are genetically-modified autologous haematopoietic stem cells.

29. The method of claim 19, wherein said transplantation of haematopoietic stem cells is for gene therapy;
optionally wherein the gene therapy is for treating a genetic haematological disease or disorder, a primary immunodeficiency or a genetic metabolic disorder, such as:
(i) a genetic haematological disease or disorder selected from a haemoglobinopathy, a transfusion dependent haemoglobinopathy, sickle cell disease, β-thalassemia major, Fanconi anaemia, and primary HLH;
(ii) a primary immunodeficiency selected from SCID, chronic granulomatous disease, and Wiskott-Aldrich syndrome; or
(iii) a genetic metabolic disorder selected from Hurler's syndrome, X-adrenoleukodystrophy, and metachromatic leukodystrophy.

30. A method of engrafting stem cells in a subject, the method comprising:
(a) administering to the subject an effective amount of the conjugate according to claim 1; and
(b) administering a stem cell population to the target tissue of the subject, wherein the administered stem cell population engrafts in the target tissue of the subject.


SEQUENCE LISTING (23 sequences)

SEQ ID NO: 1 - protein, 118 aa, Artificial Sequence
  synthetic construct
QVNLLQSGAALVKPGASVKLSCKASSYTFTDYYIHWVKQSHGKTLEWIGYINPKSGFTNYNEKFRRKATLTVDKSTNTAYMDISRLTSEDSATYYCTRRTGVIPMDAWGQGASVTVSS

SEQ ID NO: 2 - protein, 112 aa, Artificial Sequence
  synthetic construct
DVVMTQTPVSLSVSLGGQVSISCRSSQSFVSSDGNTYLNWYLQKPGQSPQLLIYKVSNRLSGVPDRFSGSGSGTDFTLKISRVEHDDLGVYYCGQASKIPLTFGSGTKLEIK

SEQ ID NO: 3 - protein, 5 aa, Artificial Sequence
  synthetic construct
DYYIH

SEQ ID NO: 4 - protein, 17 aa, Artificial Sequence
  synthetic construct
YINPKSGFTNYNEKFRR

SEQ ID NO: 5 - protein, 9 aa, Artificial Sequence
  synthetic construct
RTGVIPMDA

SEQ ID NO: 6 - protein, 16 aa, Artificial Sequence
  synthetic construct
RSSQSFVSSDGNTYLN

SEQ ID NO: 7 - protein, 7 aa, Artificial Sequence
  synthetic construct
KVSNRLS

SEQ ID NO: 8 - protein, 9 aa, Artificial Sequence
  synthetic construct
GQASKIPLT

SEQ ID NO: 9 - protein, 451 aa, Artificial Sequence
  synthetic construct
QVNLLQSGAALVKPGASVKLSCKASSYTFTDYYIHWVKQSHGKTLEWIGYINPKSGFTNYNEKFRRKATLTVDKSTNTAYMDISRLTSEDSATYYCTRRTGVIPMDAWGQGASVTVSSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 10 - protein, 219 aa, Artificial Sequence
  synthetic construct
DVVMTQTPVSLSVSLGGQVSISCRSSQSFVSSDGNTYLNWYLQKPGQSPQLLIYKVSNRLSGVPDRFSGSGSGTDFTLKISRVEHDDLGVYYCGQASKIPLTFGSGTKLEIKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 11 - protein, 123 aa, Artificial Sequence
  synthetic construct
EVQLVESGGGLVQPGGSMKLSCAASGFTFSDYYMAWVRQAPKKGLEWVASMSFAGSSTYYGDSVKGRFTISRDNAKTTLYLQMNSLRSEDTATYYCARMYTTDYYLYWYFDFWGPGTMVTVSS

SEQ ID NO: 12 - protein, 107 aa, Artificial Sequence
  synthetic construct
DVQMTQSPSYLAASPGESVSISCKASKSISNYLAWYQQKPGEANKILIYSGSTLQSGTPSRFSGSGSGTDFSLTIRNLEPEDFAVYYCQQYDEKPLTFGSGTKLEIK

SEQ ID NO: 13 - protein, 5 aa, Artificial Sequence
  synthetic construct
DYYMA

SEQ ID NO: 14 - protein, 17 aa, Artificial Sequence
  synthetic construct
SMSFAGSSTYYGDSVKG

SEQ ID NO: 15 - protein, 14 aa, Artificial Sequence
  synthetic construct
MYTTDYYLYWYFDF

SEQ ID NO: 16 - protein, 11 aa, Artificial Sequence
  synthetic construct
KASKSISNYLA

SEQ ID NO: 17 - protein, 7 aa, Artificial Sequence
  synthetic construct
SGSTLQS

SEQ ID NO: 18 - protein, 9 aa, Artificial Sequence
  synthetic construct
QQYDEKPLT

SEQ ID NO: 19 - protein, 456 aa, Rattus norvegicus
EVQLVESGGGLVQPGGSMKLSCAASGFTFSDYYMAWVRQAPKKGLEWVASMSFAGSSTYYGDSVKGRFTISRDNAKTTLYLQMNSLRSEDTATYYCARMYTTDYYLYWYFDFWGPGTMVTVSSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 20 - protein, 214 aa, Rattus norvegicus
DVQMTQSPSYLAASPGESVSISCKASKSISNYLAWYQQKPGEANKILIYSGSTLQSGTPSRFSGSGSGTDFSLTIRNLEPEDFAVYYCQQYDEKPLTFGSGTKLEIKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 21 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 22 - protein, 4 aa, Artificial Sequence
  synthetic construct
GGFG

SEQ ID NO: 23 - protein, 4 aa, Artificial Sequence
  synthetic construct
GFGG